(19) United States
(12) Patent Application Publication
(10) Pub. No.: US 2024/0058335 A1
(43) Pub. Date: Feb. 22, 2024

(54) METHODS FOR TREATMENT OF CANCER USING ABL INHIBITORS AND DRUGS TARGETING THE MEVALONATE PATHWAY

(71) Applicant: Duke University, Durham, NC (US)

(72) Inventors: Ann Marie PENDERGAST, Durham, NC (US); Jillian HATTAWAY LUTTMAN, Durham, NC (US)

(73) Assignee: Duke University, Durham, NC (US)

(21) Appl. No.: 18/270,613

(22) PCT Filed: Jan. 7, 2022
(86) PCT No.: PCT/US2022/011568

§ 371 Date: Jun. 30, 2023

Related U.S. Application Data
(60) Provisional application No. 63/134,991, filed on Jan. 8, 2021.

(51) Int. Cl. A61K 31/505; A61K 31/4439; A61P 35/00; A61K 31/366; A61K 31/405; A61K 31/506

(52) U.S. Cl. CPC A61K 31/505; A61K 31/4439; A61P 35/00; A61K 31/366; A61K 31/405; A61K 31/506

(57) ABSTRACT

The present disclosure describes, in part, methods of preventing and/or treating cancer in a subject by co-administering an ABL inhibitor and a mevalonate pathway inhibitor.

### CROSS-REFERENCE TO RELATED APPLICATION

This invention claims the benefit of priority to U.S. Provisional Application No. 63/134,991, filed Jan. 8, 2021, the contents of which are incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under Federal Grant no. R01 CA195549-01 awarded by the National Institutes of Health. The Federal Government has certain rights to this invention.

### REFERENCE TO A SEQUENCE LISTING SUBMITTED AS A TEXT FILE VIA EFS WEB

The official copy of the sequence listing is submitted electronically via EFS-Web as an ASCII formatted sequence listing with a file named 1288837_seqlist.txt, created on Jan. 5, 2022, and having a size of 3,202 bytes and is filed concurrently with the specification. The sequence listing contained in this ASCII formatted document is part of the specification and is herein incorporated by reference in its entirety.

### BACKGROUND

Metabolic reprogramming is a hallmark of cancer and contributes to tumor development. Oncogenic activation can increase expression and activity of metabolic enzymes and transporters to meet the bioenergetic and biosynthetic needs of the cancer cell thus creating metabolic vulnerabilities that might be exploited for emerging cancer therapies. Among these dependencies is mitochondrial metabolism which generates energy, regulates redox homeostasis, and provides key metabolites for macromolecule synthesis. While results from clinical trials evaluating the anticancer capability of drugs targeting mitochondrial metabolic pathways have shown potential benefits, the utility of these drugs is limited by expression of transporters that facilitate import of these drugs into cancer cells, or toxicity associated with targeting mitochondrial metabolism not only in tumor cells but also in non-cancerous tissue.

The ABL family of non-receptor tyrosine kinases, ABL1 and ABL2, are activated downstream of diverse stimuli, including oncogenic drivers such as EGFR, HER2, and KRAS, and promote progression and metastasis of solid tumor types including lung and breast cancer. ABL1 and ABL2 promote cancer cell growth, survival, adhesion, and migration depending on the cellular context. Recently, a role for ABL kinases in the regulation of mitochondria function was shown in HER2 amplified breast cancer cells as HER2 promoted mitochondrial creatine kinase 1 (MtCK1) signaling leading to cellular energy production through the mitochondrial phosphocreatine shuttle. These findings suggested that inhibition of ABL signaling may uncover additional metabolic vulnerabilities in tumor cells.

Lung cancer is the leading cause of mortality among cancers worldwide in part due to the lack of actionable targets and transient responses to current therapies.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Accordingly, one aspect of the present disclosure provides a method of treating and/or preventing a cancer in a subject, the method comprising, consisting of, or consisting essentially of administering to the subject a therapeutically effective amount of at least one ABL inhibitor and at least one mevalonate pathway inhibitor such that the cancer is treated and/or prevented in the subject.

Another aspect of the present disclosure provides a method of treating and/or preventing brain metastasis of cancer in a subject, the method comprising, consisting of, or consisting essentially of administering to the subject a therapeutically effective amount of at least one ABL inhibitor and at least one mevalonate pathway inhibitor, such that the brain metastasis is treated and/or prevented in the subject.

In some embodiments, the at least one ABL inhibitor comprises an ABL allosteric inhibitor. In some embodiment, the at least one ABL inhibitor comprises an ABL ATP-site inhibitor. In some embodiments, the at least one ABL inhibitor comprises an ABL-kinase inhibitor. In some embodiments, the ABL inhibitor is selected from the group consisting of ABL-001, imatinib, nilotinib, dasatinib (BMS-354825), bosutinib (SKI-606), Ponatinib (AP24534), Bafetinib (INNO-406), axitinib, vandertanib, GNF2, GNF5, HG-7-85-01, Tozasertib (MK-0457, VX-680), Danusertib (PHA-739358), Rebastinib (DCC-2036), 1,3,4-thiadiazole derivatives, such compound 2 having the structure

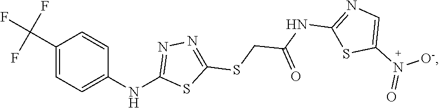

or pharmaceutically acceptable salts of any thereof, and combinations of any thereof, and pharmaceutical compositions thereof.

In some embodiments, the at least one ABL inhibitor comprises an ABL inhibitor targeting ABL protein stability. For example, in some instances, the ABL inhibitor can be a proteolysis-targeting chimera (PROTAC) compound. In some embodiments, the ABL inhibitor comprises an ABL-targeted PROTAC compound such as DAS-6-2-2-6-CRBN, BOS-6-2-2-6-CRBN, and GMB-475, or pharmaceutically acceptable salts of any thereof, and combinations of any thereof, and pharmaceutical compositions thereof.

In some embodiments, the at least one mevalonate pathway inhibitor comprises a cholesterol biosynthesis inhibitor. In some embodiments, the at least one mevalonate pathway inhibitor comprises a statin. In some embodiments, the at least one mevalonate pathway inhibitor comprises a lipophilic statin. In some embodiments, the statin is selected from the group consisting of simvastatin, atorvastatin, lovastatin, pravastatin, fluvastatin, rosuvastatin, pitavastatin, and combinations of any thereof. In some instances, the mevalonate pathway inhibitor is a cholesterol biosynthesis inhibitor.

In some embodiments, the at least one mevalonate pathway inhibitor comprises a prenylation inhibitor. For example, the prenylation inhibitor can be the GGT-1 inhibitor GGTI-298 and/or the FT inhibitor FTI-277, among other prenylation inhibitors.

In some instances, the subject is also treated with at least one of an anti-cancer agent or radiotherapy. In some embodiments, the anti-cancer agent comprises one or more of a chemotherapeutic agent, a tyrosine kinase inhibitor, or an immunotherapeutic agent.

In some instances, the subject is also treated with a cholesterol-modifying compound. The cholesterol-modifying compound can be selected from the group consisting of cholesterol efflux promoters, cholesterol import inhibitors, bile acid sequesterants, and combinations thereof.

In some embodiments, the at least one ABL kinase inhibitor is administered prior to the at least one mevalonate pathway inhibitor. In other embodiments, the at least one ABL kinase inhibitor is administered concurrently with the at least one mevalonate pathway inhibitor. In yet other embodiments, the at least one ABL kinase inhibitor is administered after the at least one mevalonate pathway inhibitor.

In some embodiments, the subject has a solid tumor cancer. In some embodiments, the subject has lung cancer. In some embodiments, the subject has breast cancer such as HER2+ breast cancer. In some embodiments, the subject has skin cancer such as melanoma. In some embodiments, the subject has solid tumor metastatic disease.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 is a schematic illustration showing the mevalonate pathway and the impact of an ABL inhibitor alone acting on mitochondrial function (e.g., ABL001, GNF5) versus an ABL inhibitor in combination with a mevalonate pathway inhibitor (e.g., statin, GGTI-298, FTI-277) according to aspects of the present disclosure.

FIGS. 2A-2G are graphs showing ABL kinase allosteric inhibitors regulate mitochondria function in lung cancer cells in accordance with aspects of the present disclosure. (FIGS. 2A-2D) Seahorse XF Analyzer Mito Stress Test analysis of mitochondrial basal respiration, maximal respiration, and ATP production as measured by changes in oxygen consumption rate (OCR). Cells were treated with GNF5 (PC9 and PC9 GR4, 10 NM; H460 and H358 15 μM), ABL001 (PC9 and PC9 GR4, 7.5 NM; H460 and H358 15 μM), Gefitinib (PC9, 15 nM; PC9 GR4, 200 nM), and Docetaxel (PC9, PC9 GR4, and H358 1 nM; H460 0.5 nM) for 24 hours. (FIG. 2E) Mitochondrial basal respiration, maximal respiration, and ATP production as measured by Seahorse XF Analyzer's Mito Stress Test for PC9 cells harboring shRNAs against ABL1/ABL2 (shAA) and non-targeting control (shSCR). (FIGS. 2F-2G) Mitochondrial ROS (MitoROS) release as measured by FACS analysis of median fluorescent intensity (MFI) using Mitosox probe. Cells were treated with 10 μM GNF5, 7.5 NM ABL001, Gefitinib (PC9, 15 nM; PC9 GR4, 200 nM), and 1 nM Docetaxel for 24 hours. Statistical analysis was performed using one-way ANOVA and Tukey post hoc testing (n=3). *p<0.05, **p<0.01, ***p<0.001, **** p<0.0001. Data are mean±SEM.

FIGS. 3A-3C are data showing metabolically focused CRISPR/Cas9 loss-of-function screen identifies inhibition of HMGCR with statin therapy as an apototic sensitizer in the presence of ABL allosteric inhibitors in accordance with aspects of the present disclosure. Genes were ranked by their log 2-transformed three score (TS) (GNF5-treated/DMSO-treated). (FIG. 3A) Validation of pharmacologic sensitization of statin therapeutics to GNF5-mediated cell death using 72 hr growth inhibition assays. Shown are sublethal doses of GNF5 (5 μM), simvastatin (1 μM), and fluvastatin (0.5 μM) derived from 72 hr dose-response curves in the presence and absence of each drug. (FIG. 3B) Immunoblots of cleaved PARP, cleaved caspase-3, and β-tubulin in PC9 cells treated for 24 hr with GNF5 (5 μM), simvastatin (1 μM), and fluvastatin (0.5 μM); (FIG. 3C) Relative change in Annexin V+PC9 cells treated for 24 hr with GNF5 (5 μM), simvastatin (1 μM), and fluvastatin (0.5 μM). Statistical analysis was performed using one-way ANOVA and Tukey post hoc testing (n=3). *p<0.05, **p<0.01, ***p<0.001, ****p<0.0001. Data are mean±SEM.

FIGS. 4A-4E are graphs showing ABL allosteric inhibitors preferentially synergize with statins to induce lung cancer cell death in accordance with aspects of the present disclosure. (FIGS. 4A-4E) Comparison of pharmacologic sensitization of statin therapeutics to ABL allosteric inhibitors versus FDA-approved therapies using 72 hr growth inhibition assays. Shown are sublethal doses of GNF5 (PC9, PC9 GR4, PC9 BrM3 5 μM; H460, H358 7.5 μM), ABL001 (PC9, PC9 GR4, PC9 BrM3 5 μM; H460, H358 7.5 μM), Gefitinib (PC9, PC9 BrM3 7.5 nM; PC9 GR4 100 nM), Docetaxel (PC9, PC9 GR4, PC9 BrM3, H358 0.5 nM; H460 0.25 nM), Simvastatin (PC9, PC9 GR4, PC9 BrM3 1 μM; H460, H358 3 μM), and Fluvastatin (PC9, PC9 GR4, PC9 BrM3 0.5 μM; H460, H358 2 μM), derived from 72 hr dose-response curves in the presence and absence of each drug. Statistical analysis was performed using one-way ANOVA and Tukey post hoc testing (n=3). *p<0.05, **p<0.01, ***p<0.001, ****p<0.0001. Data are mean±SEM.

FIGS. 5A-5F are graphs and Western blots showing mevalonate, but not cholesterol, rescues cell survival in lung cancer cells co-treated with statins and ABL allosteric inhibitors in accordance with aspects of the present disclosure. (FIG. 5A) Cell viability of PC9 GR4 cells treated with the combination of 5 μM ABL001 and 1 μM simvastatin supplemented with 500 μM mevalonate (MVA) or 25 μM cholesterol for 72 hours. Statistical analysis was performed using one-way ANOVA and Tukey post hoc testing (n=3). (FIG. 5B) Immunoblots of cleaved PARP, cleaved caspase 3, and B-tubulin in PC9 cells treated for 24 hr with 5 μM ABL001 and 1 μM simvastatin supplemented with 500 μM MVA or 25 μM cholesterol. (FIG. 5C) Cell viability of H460 cells treated with the combination of 7.5 μM ABL001 and 3 μM simvastatin supplemented with 500 μM MVA or 25 μM cholesterol for 72 hours. Statistical analysis was performed using one-way ANOVA and Tukey post hoc testing (n=3). (FIG. 5D) Immunoblots of cleaved PARP, cleaved caspase 3, and β-tubulin in H460 cells treated for 24 hr with 7.5 μM ABL001 and 3 μM simvastatin supplemented with 500 μM MVA or 25 μM cholesterol. (FIG. 5E) Immunoblots of cytochrome c, cleaved PARP, β-catenin, GAPDH, and β-Tubulin. PC9 GR4 cells were treated with of 5 μM ABL001 and 1 μM simvastatin supplemented with 500 μM MVA for 24 hr, collected, and fractionated. Cytoplasmic, membrane, and whole cell fractions are shown for drug treatments. (FIG. 5F) qRT-PCR of indicated mRNAs in PC9 GR4 cells treated with 5 μM ABL001 and 1 μM simvastatin supplemented with 500 μM MVA or 25 NM cholesterol for 24 hours. Statistical analysis was performed using one-way ANOVA and Fisher post hoc testing (n=3). *p<0.05, **p<0.01, ***p<0.001, ****p<0.0001. Data are mean±SEM.

FIGS. 6A-6E are graphs and Western blots showing apoptotic sensitization to statin therapy by ABL allosteric inhibitors requires inhibition of protein prenylation in accordance with aspects of the present disclosure. (FIG. 6A) Cell viability of PC9 GR4 cells treated with the combination of 5 NM ABL001 and 1 NM simvastatin supplemented with 500 NM MVA, 10 NM FPP, or 10 μM GPP for 72 hours. (FIG. 6B) Immunoblots of unprenylated RAP1A, HDJ-2, and β-tubulin in PC9 GR4 cells treated for 24 hr with 5 μM ABL001 and 1 NM simvastatin supplemented with 500 NM MVA, 10 μM FPP (farnesylation metabolite), or 10 μM GPP (geranylgeranylation metabolite). Simvastatin caused a mobility shift of HDJ-2 (slower, migrating unprenylated form) and induced the appearance of unprenylated RAP1A. Prenylation alterations were rescued with the indicated metabolites for each pathway. (FIG. 6C) Cell viability of PC9 GR4 cells treated 5 NM ABL001 and 1 NM simvastatin, 500 NM MVA, 5 NM GGTI-298 (GGT inhibitor), 12.5 NM FTI-277 (FT inhibitor) for 72 hr. n=3 (FIG. 6D) Immunoblots of unprenylated RAP1A, HDJ-2, and β-tubulin in PC9 GR4 cells treated for 24 hr with 5 NM ABL001 and 1 μM simvastatin supplemented with 500 NM MVA, 5 NM GGTI-298, and 12.5 NM FTI-277. (FIG. 6E) Mitochondrial respiration changes in the cells as treated in FIG. 6C. Statistical analysis was performed using one-way ANOVA and Tukey post hoc testing (n=3). **p<0.01, ***p<0.001, ****p<0.0001. Data are mean±SEM.

FIGS. 7A-7E are graphs, and images showing combination therapy of ABL001 and simvastatin promotes tumor cell apoptosis and increases survival in mouse models of lung cancer brain metastasis and gefitinib resistance in accordance with aspects of the present disclosure. PC9 BrM3 or PC9 GR4 labeled with luciferase-tomato (pFULT) were intracardially injected into mice on Day 0. IVIS imaging of metastatic burden was performed on Day 6 followed by equivalent stratification of mice into treatment groups based on tumor flux. Oral gavage treatment of drugs began on Day 7 followed by weekly IVIS imaging until experimental end point (Day 50). Statistical analysis of overall survival was calculated using log rank (MantelCox) test and interactions below an adjusted p<0.017 were deemed significant accounting for 3 pairwise comparisons. (FIG. 7A) Overall survival of mice injected intracardially with PC9 BrM3-pFULT cells treated with DMSO (n=16), ABL001 (n=19), simvastatin (n=17), and combination (n=19). (FIG. 7B) Overall survival of mice injected intracardially with PC9 GR4-pFULT cells treated with DMSO (n=9), ABL001 (n=9), simvastatin (n=9), and combination (n=9). (FIG. 7C) Quantitative analysis (day 30 post-injection) of whole body metastatic index in mice injected intracardially with PC9 BrM3 cells and treated with DMSO (n=16), ABL001 (n=19), simvastatin (n=17), and combination (n=19) and representative tumor imaging in mice 27 days post-IC injection. (FIGS. 7D-7E) Quantification of percent positive proliferative (FIG. 7D) and apoptotic (FIG. 7E) nuclei in tumor sections. Statistical analysis was performed using one-way ANOVA with Dunnet post hoc testing (n=3).*p<0.05, **p<0.01. Data are mean±SEM.

FIGS. 8A-8G are results showing ABL allosteric inhibitors, but not ATP-competitive inhibitor, impair mitochondria function in lung cancer cells in accordance with aspects of the present disclosure. Seahorse XF Analyzer Mito Stress Test analysis of mitochondrial basal respiration, maximal respiration, and ATP production as measured by changes in oxygen consumption rate (OCR) in the indicated lung cancer cells: (FIG. 8A) PC9; (FIG. 8B) PC9 GR4; (FIG. 8C) H460. Cells were treated with GNF5 (PC9 and PC9 GR4, 10 NM; H460 15 μM) and Nilotinib (PC9, PC9 GR4, H460 2 μM) for 24 hours (FIGS. 8A-8C). Doses correspond to IC50 dose for each drug in the corresponding cell line. In each graph, for each of Basal Respiration, Maximal Respiration, and ATP production, data is shown from left to right for DMSO, GNF5, and Nilotinib. (FIGS. 8D-8E) The same stress test analysis as in FIGS. 8A-8C in PC9 cells treated with DMSO, GNF5, ABL001, Niotinib, Gefitinib, or Docetaxel (from left to right) for 48 hr and 72 hr, respectively. (FIGS. 8F-8G) Seahorse XF Analyzer Glycolytic Rate analysis of basal and compensatory glycolysis in PC9 and PC9 GR4 cells. Cells were treated with IC50 doses for 24 hours (n=3). Data are mean±SEM. Statistical analysis was performed using one-way ANOVA and Tukey post hoc testing *p<0.05, **p<0.01.

FIGS. 9A-9I are results showing ABL inhibition impacts mitochondria function in lung cancer cells without altering mitochondria number in accordance with aspects of the present disclosure. (FIG. 9A) Mitochondrial basal respiration, maximal respiration, and ATP production as measured by changes in oxygen consumption rate (OCR) from Seahorse XF Analyzer Mito Stress Test analysis for H460 cells harboring shRNAs against ABL1/ABL2 (shAA) and non-targeting control (shSCR). (FIG. 9B) FACS analysis of mitochondrial ROS (MitoROS) using mitosox probe in H460 cells treated with 15 NM GNF5, 15 NM ABL001, and 0.5 nM Docetaxel for 24 hours. n=3 (FIGS. 9C-9D) MitoROS in H460 AA knockdown cells (FIG. 9C) or PC9 AA knockdown cells (FIG. 9D). (FIGS. 9E-9F) Mitotracker staining to analyze mitochondria number in H460 cells with 15 NM GNF5 (FIG. 9E) or PC9 cells treated with 10 NM GNF5 (FIG. 9F). n=3 (FIGS. 9G-9H) Mitotracker staining of H460 AA knockdown cells (FIG. 9G) or PC9 AA knockdown cells (FIG. 9H). n=3 Statistical analysis was calculated using unpaired two-tailed t test *p<0.05, Data are mean±SEM. (FIG. 9I) H460 cells stained with Mitotracker and Hoescht 33342 following treatment with IC50 dose for each drug for 24 hours. Image quantification showing mitochondrial length×width plotted for thousands of mitochondria from >10 cells across at least two independent experiments. Changes were not statistically significant.

FIGS. 10A-10B are results showing knockdown of HMGCR sensitizes lung cancer cells to cell death in the presence of ABL allosteric inhibitor in accordance with aspects of the present disclosure. (FIG. 10A) Quantification of absorbance reading of colony formation assays of PC9 GR4 and H460 cells treated with ABL001 (1 μM), simvastatin (100 nM), and combination of ABL001 and simvastatin for 1 week (n=2). Data are mean±SEM. (FIG. 10B) PC9 cells harboring two distinct shRNAs (#46448 and #46452) against HMGCR or non-targeted control (NTC) were treated with GNF5 for 72 hr to assess cell viability.

FIGS. 11A-B are results showing ATP-competitive inhibitor Nilitonib fails to sensitize lung cancer cells to statin therapy in accordance with aspects of the present disclosure. (FIGS. 11A-11B) Cell viability assays were used to examine sensitization of statin therapeutics to the ABL ATP-site inhibitor Nilotinib after 72 hr of co-treatment in the indicated lung cancer cells. Sublethal doses of Nilotinib (PC9 GR4, 5 NM; H460, 7.5 μM), simvastatin (PC9 GR4, 1 NM; H460 3 μM), and fluvastatin (PC9 GR4, 0.5 NM; H460 2 μM), were used and cell viability assays were performed after 72 hr with indicated drugs.

FIGS. 12A-12C are results showing combination treatment of subtherapeutic doses of statins with IC50 value dose for ABL allosteric inhibitors, Gefitinib, or Docetaxel, demonstrate preferential synergy with ABL allosteric inhibitors in accordance with aspects of the present disclosure. (FIGS. 12A-12B) Cell viability assays to evaluate sensitization of statin therapeutics to ABL allosteric inhibitors versus FDA-approved therapies at IC50 doses using 72 hr growth inhibition assays. Shown are IC₅₀ doses of GNF5 (PC9, PC9 GR4 10 μM), ABL001 (PC9, PC9 GR4 7.5 μM), Gefitinib (PC9, 15 nM; PC9 GR4 200 nM), Docetaxel (PC9, PC9 GR4 1 nM). Simvastatin was used at subtherapeutic doses (PC9, PC9 GR4 1 μM). (FIG. 12C) Corresponding western blot analysis of cleaved PARP, total PARP, cleaved caspase 3, total caspase, and β-Tubulin in PC9 cells treated with IC50 doses of ABL001, Gefitnib, and Docetaxel combined with subtherapeutic doses of simvastatin.

FIGS. 13A-13D are results showing inhibition of protein prenylation sensitizes lung cancer cells to ABL allosteric inhibitor in accordance with aspects of the present disclosure. (FIG. 13A) Cell viability of PC9 cells treated with the combination of 5 μM ABL001 and 1 μM simvastatin supplemented with 500 μM MVA, FPP, or GPP for 72 hours. (FIG. 13B) Cell viability of H460 cells treated with the combination of 7.5 μM ABL001 and 3 μM simvastatin supplemented with 500 μM MVA, FPP, or GPP for 72 hours. (FIG. 13C) Cell viability of PC9 GR4 cells treated 5 μM ABL001 and 1 μM simvastatin, 500 μM MVA, 5 μM GGTI-298, 12.5 μM FTI-277 for 72 hr. (FIG. 13D) Cell viability of H460 cells treated with the combination of 7.5 RM ABL001, 3 μM simvastatin, 500 μM MVA, 3 μM GGTI-298, 3 μM FTI-277 for 72 hr. Statistical analysis was performed using one-way ANOVA and Tukey post hoc testing (n=3). **p<0.01, ***p<0.001, ****p<0.0001. Data are mean±SEM.

FIGS. 14A-14C show graphs illustrating that combination therapy of ABL001 and simvastatin does not significantly impair growth of lung tumor xenografts implanted subcutaneously or orthotopically in the lung according to certain aspects of this disclosure. (FIG. 14A) Mice were subcutaneously injected with PC9 GR4 cells and stratified into four treatment groups once tumors reached 50 mm3. Mice were treated for a total of 3 weeks and tumor volume was measured weekly. n=7 mice per group. Statistical analysis performed via one-way ANOVA. Quantification of tumors was normalized to the average flux on Day 1 of treatment for all tumors within each treatment arm; ns=not significant. (FIG. 14B) Timecourse quantification of PC9 tumor growth in athymic nude mice injected orthotopically via lung intrathoracic injection and treated with drugs as indicated. Dosing began 6 days post-injection and randomization of mice into treatment groups, followed by weekly bioluminescent imaging. Treatment continued for 36 days; n=9 mice per group. Statistical analysis performed via one-way ANOVA. Quantification of tumors was normalized to the average Day 0 flux for all tumors within each treatment arm; ns=not significant. (FIG. 14C) Quantification of tumor burden as measured via bioluminescent imaging on Day 42 post-intrathoracic injection of PC9 pFuLT-expressing tumor cells as in S6C. n=9 mice per group. Statistical analysis performed via one-way ANOVA. Tumor flux was normalized to the average Day 0 flux for all tumors within each treatment arm.

FIGS. 15A-15F show various data illustrating pharmacologic sensitization of statin therapeutics to ABL allosteric inhibitor-mediated cell death according to certain aspects of this disclosure. Cells were treated with sublethal doses of GNF5 (5 μM), ABL001 (5 μM), simvastatin (1 μM), and fluvastatin (0.5 μM) in the presence and absence of each drug (n=3) for 72 hrs. (FIG. 15A) Cell viability following 3 day treatment. (FIG. 15B) Synergy scores for cell survival using the Bliss Formula of synergy (1.0 is equivalent to true synergy); n=3 biological replicates for each experiment. (FIG. 15C) Blots for cleaved PARP and caspase 3, and indicated proteins. (FIG. 15E) Western blots of HCC1954 HER2+ breast cancer cells treated with ABL001, Statin, or combination therapy. Cell viability was evaluated with Cell Titer Glo;***p<0.005; NS=not significant. Data are mean±SEM.

FIGS. 16A-16B show that ABL kinase inhibition impairs outgrowth of HER2+ breast cancer brain metastatic cells and improve overall survival of tumor-bearing mice according to certain aspects of this disclosure. Nude mice were injected intracranially with HCC1954-LCC1 cells on Day 0. On Day 10, mice were divided evenly between treatment groups (Vehicle or GNF5) based on tumor burden and weight. Tumors were monitored weekly by BLI until experimental endpoint (Day 55). (FIG. 15A) Overall survival curve of nude mice injected intracranially with HCC1954-LCC1 and treated with vehicle (n=9) or GNF5 (n=10) and representative BLI images of mice at day 33 post-injection. (FIG. 16B) Overall survival curve of nude mice injected intracranially with HCC1954-LCC1 ABL1/2 KO (n=15) or control cells (n=15) and representative of BLI images of mice at day 21 post-injection. Statistical analysis was performed using Log-rank (Mantel-Cox) test.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless otherwise defined, all technical terms used herein have the same meaning as commonly understood by one of ordinary skill in the art to which this disclosure belongs.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

### I. Introduction

Provided herein are methods of treating and/or preventing cancer in a subject by administering a co-therapy of an ABL inhibitor and at least one of mevalonate pathway inhibitor. The present disclosure is based, in part, on the findings by the inventors demonstrating that mevalonate pathway inhibitors synergize with ABL inhibitors to promote cancer cell death. Thus, provided herein are methods of treating cancer comprising co-administration of an ABL inhibitor and a mevalonate pathway inhibitor.

The mevalonate pathway, also known as the isoprenoid pathway or HMG-CoA reductase pathway, is an essential metabolic pathway present in eukaryotes, archaea, and some bacteria. The pathway begins with acetyl-CoA and ends with the production of isopentenyl pyrophosphate (IPP) and dimethylallyl pyrophosphate (DMAPP), which are used to make isoprenoids, a diverse class of biomolecules such as cholesterol, vitamin K, coenzyme Q10, and all steroid hormones. The mevalonate pathway is best known as the target of statins, a class of cholesterol lowering drugs. Statins inhibit HMG-CoA reductase within the mevalonate pathway.

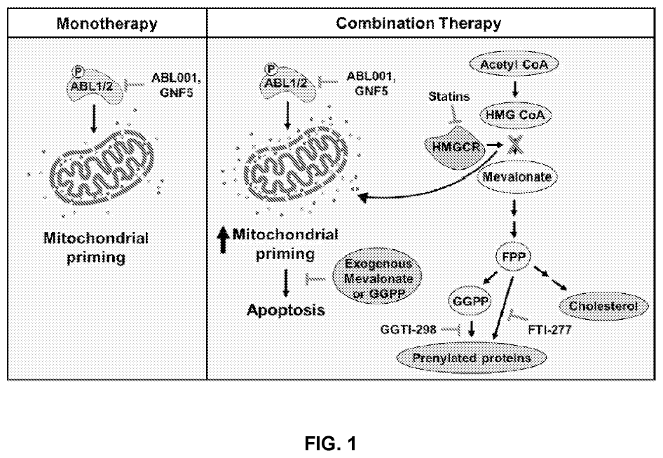

As shown in FIG. 1, mevalonate is the precursor to farnesyl diphosphate (FPP), which can either be converted to geranylgeranyl diphosphate (GGPP) or cyclized to produce squalene for cholesterol production. Both FPP and GGPP are metabolites in the isoprenoid pathway required for protein prenylation, a posttranslational enzymatic modification that adds a prenylated motif to CAAX proteins, such as the RAP1A GTPase. These modifications regulate protein localization to different cellular compartments, facilitate specific protein-protein interactions and modulate protein stability.

Metabolic reprogramming in tumors is an adaptation that allows cancer cells to meet enhanced bioenergetic needs, but metabolic dysregulation also generates vulnerabilities in cancer cells that can be exploited for the development of treatment strategies. Among these vulnerabilities is mitochondrial oxidative metabolism as cancer cells are reliant on functional mitochondria for malignant transformation and growth. As described herein, the inventors determined that, in comparison to current FDA-approved therapeutics, gefitinib and docetaxel, ABL allosteric inhibitors markedly decrease mitochondria function in lung cancer cells.

The present disclosure is based, in part, on the discovery by the inventors that ABL kinases regulate mitochondrial function and integrity in lung adenocarcinoma cells harboring EGFR and KRAS mutations, and that inactivation of ABL kinases impairs oxidative mitochondrial metabolism. As ABL inhibition impairs mitochondrial oxidation, it was sought to determine whether targeting metabolic pathways could enhance sensitivity to ABL allosteric inhibitors by performing a CRISPR/Cas9 loss-of-function screen targeting 2,322 metabolic enzymes and transporters. This screen identified HMG-CoA reductase (HMGCR), a rate-limiting enzyme of the mevalonate pathway and target of statin therapy, as a top-scoring sensitizer capable of potentiating cell death in the presence of sublethal doses of ABL allosteric inhibitors. Thus, the inventors identified dual inactivation of the mevalonate pathway and ABL kinases as a strategy to augment apoptotic cell death and enhance therapeutic efficacy.

Notably, it was found that combination therapy of ABL kinase allosteric inhibitors with lipophilic statins impaired growth of clinically relevant therapy-resistant lung cancer cells and brain metastatic lung cancer cells in vitro and in in vivo mouse models. Patients with lung cancer have the highest leading cancer-related mortality worldwide in part due to the lack of durable responses to current therapies resulting in metastatic and therapy-resistant disease progression.

Dysregulation of the mevalonate pathway has been implicated in the progression of solid tumors including glioblastoma, breast, and liver cancer. Cancer cells exploit distinct bioactive end-products generated by the mevalonate pathway, including cholesterol and isoprenoid intermediates, to promote tumor progression and therapy resistance. For example, glioblastomas rely on exogenous cholesterol for survival and cholesterol depletion induces glioblastoma cell death. In contrast, it was found that the synergistic interaction between ABL allosteric inhibitors and statins appears to be mediated by inhibition of protein prenylation and is independent of decreased cholesterol. Specifically, metabolic rescue of the geranylgeranylation pathway, but not cholesterol, was capable of rescuing cell survival in lung cancer cells co-treated with ABL001 and statins to an extent equivalent to mevalonate.

Upon finding that the downstream sterol metabolite cholesterol did not rescue cell survival, the inventors investigated whether metabolites in the isoprenoid pathway were critical for sensitization to statin therapeutics. It was found that addition of GGPP preferentially rescued cell viability compared to FPP in PC9 GR4, PC9, and H460 cells co-treated with ABL001 and simvastatin. Thus, without being held to any particular theory, inhibition of either geranylgeranyl transferase (GGT) or farnesyl transferase (FT) could impact cell survival in a manner similar to simvastatin treatment in the presence of ABL allosteric inhibitors. Survival of PC9 GR4, PC9, and H460 lung cancer cells co-treated with ABL001 and the GGT-1 inhibitor (GGTI-298) was significantly impaired, but cell survival was only slightly decreased following addition of the FT inhibitor (FTI-277) in the presence of ABL001. Without being held to any particular theory, the non-additivity observed for statin treatment and GGT and FT inhibition suggests that the synergizing effects of statins or GGTI-298+FTI-277 in the presence of ABL allosteric inhibitor may operate through the same pathway. Immunoblotting confirmed that each inhibitor specifically suppressed its target pathway. Collectively, these data revealed that inhibition of protein geranylgeranylation is sufficient to sensitize cells to ABL allosteric inhibitors leading to enhanced intrinsic apoptosis. FIG. 1 illustrates the impact of the combination therapy compared to ABL inhibitor monotherapy on the mevalonate pathway.

Protein geranylgeranylation is required for processes such as protein and vesicular trafficking, and cell proliferation. Multiple geranylgeranylated proteins might be targeted by statins in ABL-depleted lung cancer cells. A recent report showed that lipophilic statins prevent membrane association of Rab11b, a small GTPase that regulates endosomal recycling, and decreases breast cancer brain metastasis in mice. Among numerous substrates of the geranylgeranylation pathway are RAS-related GTPases, including members of the RAS and RHO-RAC families, which can function to regulate lung cancer cell survival in vitro and metastasis in mice.

ABL kinases can target multiple substrates in cancer cells to promote cytoskeletal alterations, organelle trafficking, cell growth and cell survival. As described herein, the inventors determined that ABL inactivation impairs mitochondria function and organelle integrity following pharmacologic inhibition or genetic depletion, which are not induced by treatment with gefitinib or docetaxel. These findings suggest that sensitization to statin therapy might be mediated through mitochondrial priming triggered by ABL kinase inhibition. Statins have also been shown to inhibit synthesis of ubiquinone and coenzyme Q, critical components of the electron transport chain (ETC), through impeding mevalonate production. Previous reports showed that statins can enhance mitochondrial priming and sensitize cancer cells to mitochondrial-mediated apoptosis. For example, inhibition of the pro-survival factor BCL-2 sensitized leukemia cells to statin therapeutics promoting apoptosis. Future studies are needed to assess whether ABL kinase inhibition impairs mitochondria by altering the activity of the ETC. In this regard, Src family tyrosine kinases have been shown to phosphorylate subunits of the ETC resulting in subsequent changes in ETC complex activity, and inhibition of Src kinases results in decreases in complex I activity and decreased mitochondrial respiration. ABL1 has been shown to be activated downstream of oncogenic Src. Without being held to any particular theory, it is possible that combination treatment of ABL allosteric inhibitors and statins can impair one or more complexes of the ETC, thereby augmenting mitochondrial-mediated apoptosis.

Previous reports have identified the potential of statins to function as anticancer agents; however, clinical trials using various chemotherapies in combination with statins have had either marginal or no effect on distant metastasis-free survival or overall survival in lung cancer patients with advanced disease. (Han, J. Y., et al., Clin. Cancer Res. 17:1553-1560 (2011); Lee, Y., et al., Cancer Res. Treat. 49:1001-1011 (2017); Seckl, M. J., et al., J. Clin. Oncol. 35:1506-1514 (2017).) Retrospective analyses of various lung cancer patient cohorts have reported mixed findings on the impact of statin therapeutics on cancer related mortality for patients taking statins at the onset of chemotherapy treatment. (Cardwell, C. R., et al., Cancer Epidemiol. Biomarkers Prev. 24:833-841 (2015); Khurana, V., et al., Chest 131:1282-1288 (2007); Kuoppala, J., et al., Eur. J. Cancer 44:2122-2132 (2008); Wang, J., et al., PLoS ONE 8:e77950 (2013).) The findings provided herein are consistent with clinical reports showing that statins added to first-line standard of care chemotherapy do not impact lung adenocarcinoma progression and provide use of ABL allosteric inhibitors in combination with statins or other ABL inhibitors for the treatment of lung cancer patients with advanced disease.

Whereas inactivation of ABL kinases impairs breast and lung cancer metastasis in mouse models, clinical trials to treat breast and lung cancer patients with ABL ATP-site inhibitors have been ineffective in part due to targeting of multiple kinases other than ABL, possibly leading to paradoxical activation of cell survival pathways. Notably, recent work by the inventors revealed that ABL allosteric inhibitors, but not ABL ATP-competitive inhibitors, disrupt the interaction between ABL2 and HSF1, a transcription factor that promotes lung cancer growth and metastatic colonization of the brain. (Hoj, J. P., et al., Proc. Natl. Acad. Sci. USA 117:33486-33495 (2020).) This finding suggests that protein-protein interactions dependent on distinct ABL protein conformations are disrupted by the binding of the allosteric inhibitors to a unique site in the ABL kinase domain. In alignment with these findings, our work shows that the ABL allosteric inhibitors, which bind to the myristoyl-binding pocket in the C-lobe of the ABL kinase domain and are highly selective inhibitors of the ABL kinases, are capable of impairing mitochondria function in a manner similar to genetic inhibition of the ABL kinases, whereas the ATP-competitive inhibitors do not. Thus, the findings provided herein support the treatment methods using ABL allosteric site inhibitors in combination with statins or other ABL inhibitors as a treatment strategy for lung cancer patients with advanced disease, including those patients with difficult to treat brain metastases or EGFR TKI resistance, and other solid tumors.

### II. Methods

Accordingly, one aspect of the present disclosure provides a method of treating and/or preventing a cancer in a subject, the method comprising, consisting of, or consisting essentially of administering to the subject a therapeutically effective amount of at least one ABL inhibitor and at least one mevalonate pathway inhibitor such that the cancer is treated and/or prevented in the subject.

Another aspect of the present disclosure provides a method of treating and/or preventing brain metastasis of cancer in a subject, the method comprising, consisting of, or consisting essentially of administering to the subject a therapeutically effective amount of at least one ABL inhibitor and at least one mevalonate pathway inhibitor such that the brain metastasis is treated and/or prevented in the subject.

As used herein, “treatment,” “therapy” and/or “therapy regimen” refer to the clinical intervention made in response to a disease, disorder or physiological condition (e.g., a cancer) manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject (e.g., cancer), who does not have, but is at risk of or susceptible to developing a disease, disorder or condition.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The methods of the present disclosure can be used to treat cancer and metastases thereof. In some embodiments, the methods provided herein are used to treat a solid tumor cancer in a subject. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer (such as melanoma), brain cancer, neuroblastoma, myeloma, and various types of head and neck cancer. In some embodiments, the cancer is characterized by ABL dysfunction, mutation, and the like. In some instances, the subject has a primary tumor. In some instances, the subject has a recurrent cancer (e.g., following primary diagnosis and treatment). In some instances, the subject has recurrent cancer due to development of resistance to the therapeutic agent administered as the prior treatment.

In some embodiments, the subject has lung cancer such as, for example, non-small cell lung cancer, small cell lung cancer, mesothelioma, carcinoid tumors, or lung adenocarcinoma. In some embodiments, the subject has lung cancer comprising an oncogenic mutation in epidermal growth factor receptor (EGFR, also known as ERBB1 and HERD. The EGFR mutant lung cancer can be sensitive to EGFR tyrosine kinase inhibitors (TKIs) or can be TKI-resistant. In some embodiments, the subject has a KRAS mutant lung cancer. In some embodiments, the subject has large cell lung cancer (LCC). In some embodiments, the subject has KRAS mutant large cell lung carcinoma. In some embodiments, the subject has KRAS mutant lung adenocarcinoma.

In some embodiments, the subject has breast cancer. Exemplary breast cancers include triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors. The breast cancer can be Human Epidermal Growth Factor Receptor-2 (HER2) positive (HER2⁺) breast cancer or HER2 negative (HER2⁻) breast cancer. A breast cancer is considered to be HER2-negative (HER2⁻) if it does not detectably express HER2 whereas a breast cancer is determined to be HER2-positive (HER2⁺) if it does detectably express HER2. The breast cancer can be estrogen receptor positive (ER⁺) or ER negative (ER⁻). A breast cancer is considered to be ER⁻ if it does not detectably express ER, whereas a breast cancer is determined to be ER⁺ if it does detectably express ER. The breast cancer can be progesterone receptor positive (PR⁺) or PR negative (PR⁻). A breast cancer is considered to be PR⁻ if it does not detectably express PR, whereas a breast cancer is determined to be PR⁺ if it does detectably express PR. Detectable expression of HER2, ER, and PR is determined by evaluating protein expression, typically by immunohistochemistry. In some instances, the breast cancer is triple negative (ER-negative, PR-negative, and HER2-negative) breast cancer. In some instances, the breast cancer is HER2 positive breast cancer.

In some embodiments, the subject has skin cancer. The skin cancer can be basal cell carcinoma, squamous cell carcinoma, melanoma, dermatofibrosarcoma, Kaposi sarcoma, Merkel cell carcinoma, or sebaceous gland carcinoma. In some instances, the cancer is melanoma. Melanoma is a form of skin cancer that begins in the cells (melanocytes) that control the pigment in skin. The staging system most often used for melanoma is the American Joint Committee on Cancer (AJCC) TNM system. The TNM system is based on three pieces of information: tumor thickness, ulceration, and metastasis to lymph nodes. Once a subject's T, N, and M categories have been determined, this information is combined in a process called stage grouping to assign an overall stage. The staging system generally uses the pathologic stage (also called the surgical stage) that is determined by examining tissue removed during an operation but, sometimes, if surgery is not possible right away (or at all), the cancer will be given a clinical stage based on the results of physical exams, biopsies, and imaging tests instead.

In some embodiments, the methods provided herein are used to treat solid tumor metastatic disease in a subject. In some embodiments, the subject has lung cancer brain metastasis. In some embodiments, the subject has breast cancer brain metastasis. In some embodiments, the subject has skin cancer brain metastasis such as metastasis from melanoma.

Any compound suitable for inhibiting the function, expression, and/or activity of the ABL kinase can be used in the methods provided herein including, but not limited to, allosteric inhibitors, ABL ATP-site inhibitors, ABL-kinase inhibitors, and the like. In some embodiments, the ABL inhibitor is selected from the group consisting of, ABL-001, imatinib, nilotinib, dasatinib (BMS-354825), bosutinib (SKI-606), Ponatinib (AP24534), Bafetinib (INNO-406), axitinib, vandertanib, GNF2, GNF5, HG-7-85-01, Tozasertib (MK-0457, VX-680), Danusertib (PHA-739358), Rebastinib (DCC-2036), 1,3,4-thiadiazole derivatives, such compound 2 having the structure

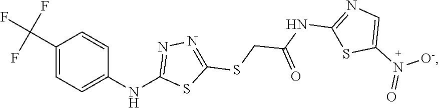

or pharmaceutically acceptable salts of any thereof, and combinations of any thereof and pharmaceutical compositions thereof. See Luttman et al., Cell Commun. Signal 19:59 (2021), which is incorporated herein in its entirety for all purposes.

In some embodiments, the at least one ABL inhibitor comprises an ABL inhibitor targeting ABL protein stability. For example, in some instances, the ABL inhibitor can be a proteolysis-targeting chimera (PROTAC) compound. In some embodiments, the ABL inhibitor comprises an ABL-targeted PROTAC compound such as DAS-6-2-2-6-CRBN, BOS-6-2-2-6-CRBN, and GMB-475, or pharmaceutically acceptable salts of any thereof, and combinations of any thereof, and pharmaceutical compositions thereof. See Luttman et al., Cell Commun. Signal 19:59 (2021).

In some instances, the at least one mevalonate pathway inhibitor comprises a cholesterol biosynthesis inhibitor. In some instances, the mevalonate pathway inhibitor comprises a lipophilic mevalonate pathway inhibitor. In some embodiments, the mevalonate pathway inhibitor comprises a statin. In some embodiments, the mevalonate pathway inhibitor comprises a lipophilic statin. In some embodiments, the statin is selected from the group consisting of simvastatin (Zocor®), atorvastatin (Lipitor®), lovastatin (Mevacor®), pravastatin (Pravachol®), Fluvastatin (Lescol®), rosuvastatin (Crestor), pitavastatin (Livalo®), and combinations of any thereof.

In some embodiments, the mevalonate pathway inhibitor comprises a prenylation inhibitor. For example, the prenylation inhibitor can be the GGT-1 inhibitor GGTI-298 and/or the FT inhibitor FTI-277, among other prenylation inhibitors.

### III. Dosing and Administration

The ABL inhibitors and mevalonate pathway inhibitors, and pharmaceutical compositions thereof, as described herein can be administered to a subject by any technique known in the art, including local or systemic delivery. In some embodiments, the at least one ABL inhibitor and the at least one mevalonate pathway inhibitor are administered orally. As used herein, the term “administering” an agent, such as a therapeutic agent/entity to a subject or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the subject, including, but not limited to, delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

Methods for administration of therapeutic agents are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., PA.). The ABL inhibitors and mevalonate pathway inhibitors, and pharmaceutical compositions thereof, can each be administered in a single dose or in multiple doses (e.g., two, three, or more single doses per treatment) over a time period (e.g., hours or days).

Co-administration need not refer to administration at the same time in an individual, but rather may include administrations that are spaced by hours or even days, weeks, or longer, as long as the administration of the one or more therapeutic agents is the result of a single treatment plan. The co-administration may comprise administering the ABL inhibitor of the present disclosure before, after, or at the same time as the mevalonate pathway inhibitor or other therapeutic agent. By way of example, the at least one ABL inhibitor may be given as an initial dose in a multi-day protocol, with the at least one mevalonate pathway inhibitor given on later administration days; or the at least one mevalonate pathway inhibitor can be given as an initial dose in a multi-day protocol, with the at least one ABL inhibitor given on later administration days. On another hand, one or more mevalonate pathway inhibitors and ABL inhibitor(s) as described herein may be administered on alternate days in a multi-day protocol. In still another example, a mixture of one or more mevalonate pathway inhibitors and one or more ABL inhibitors as described herein may be administered concurrently. This is not meant to be a limiting list of possible administration protocols.

The term “effective amount” or “therapeutically effective amount” refers to an amount sufficient to effect beneficial or desirable biological and/or clinical results.

An effective amount for a particular subject/patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

An effective amount of a therapeutic agent (e.g., ABL inhibitor, mevalonate pathway inhibitor, etc.) is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

Determination of an effective dosage of the at least one ABL inhibitor and/or the at least one mevalonate pathway inhibitor for a given mode of administration is well within the capabilities of those skilled in the art. Effective dosages may be estimated initially from in vitro activity and metabolism assays. For example, an initial dosage of the therapeutic agent for use in animals may be formulated to achieve a circulating blood or serum concentration that is at or above an IC50 of the particular agent as measured in an in vitro assay. The dosage can be calculated to achieve such circulating blood or serum concentrations taking into account the bioavailability of the particular agent via the desired route of administration. Initial dosages of compound can also be estimated from in vivo data, such as animal models. For example, an average mouse weighs 0.025 kg. Administering 0.025, 0.05, 0.1 and 0.2 mg of an agent per day may therefore correspond to a dose range of 1, 2, 4, and 8 mg/kg/day. If an average human adult is assumed to have a weight of 70 kg, the corresponding human dosage would be 70, 140, 280, and 560 mg of the agent per day. Dosages for other active agents may be determined in similar fashion. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages suitable for human administration.

Exemplary daily dosages for various statins are shown in Table 1 below (exceptions noted), with doses based on percent reduction in low-density lipoprotein cholesterol (LDL-C) desired for subject. NA=not applicable. See also Stone, N.J., et al., 2013 “ACC/AHA Guideline on the Treatment of Blood Cholesterol to Reduce Atherosclerotic Cardiovascular Risk in Adults: A Report of the American College of Cardiology/American Heart Association Task Force on Practice Guidelines.” J Am Coll Cardiol. 2013; 63(25 Pt B):2889-934.

TABLE 1
Statin Dosages

| | Dosage | | |
|---|---|---|---|
| | | Moderate-intensity | |
| | Low-intensity | (LDL-C reduction 30% | High-intensity |
| Statin | (LDL-C reduction <30%) | to <50%) | (LDL-C reduction >50%) |
| Atorvastatin | NA | 10 to 20 mg | 40 to 80 mg |
| Fluvastatin | 20 to 40 mg | 40 mg 2×/day; XL 80 mg | NA |
| Lovastatin | 20 mg | 40 mg | NA |
| Pitavastatin | 1 mg | 2 to 4 mg | NA |
| Pravastatin | 10 to 20 mg | 40 to 80 mg | NA |
| Rosuvastatin | NA | 5 to 10 mg | 20 to 40 mg |
| Simvastatin | 10 mg | 20 to 40 mg | NA |

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The at least one ABL inhibitor and/or the at least one mevalonate pathway inhibitor, and pharmaceutical compositions thereof, if desired, can be presented in a pack or dispenser device which may contain one or more unit dosage forms containing the at least one ABL inhibitor and/or the at least one mevalonate pathway inhibitor. The pack may, for example, comprise metal or plastic foil, such as a blister pack. The pack or dispenser device may be accompanied by instructions for administration.

Other aspects of the present disclosure provides a kit for the treatment of pain comprising, consisting of, or consisting essentially of a therapeutically effective amount of the at least one ABL inhibitor and/or the at least one mevalonate pathway inhibitor as provided herein, an apparatus for administering the at least one ABL inhibitor and/or the at least one mevalonate pathway inhibitor, and instructions for use. In some embodiments, the kit further provides at least one additional therapeutic agent as provided herein and an apparatus for administering the at least one additional therapeutic to the subject.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

“Contacting” as used herein, e.g., as in “contacting a sample” refers to contacting a sample directly or indirectly in vitro, ex vivo, or in vivo (i.e. within a subject as defined herein). Contacting a sample may include addition of a compound to a sample, or administration to a subject. Contacting encompasses administration to a solution, cell, tissue, mammal, subject, patient, or human. Further, contacting a cell includes adding an agent to a cell culture.

### IV. Co-Therapies

The methods provided herein provide for the co-administration of at least one ABL inhibitor and at least one mevalonate pathway inhibitor. In some embodiments, the subject may also be receiving additional therapeutic agents such as anti-cancer therapies and/or treatment with a cholesterol-modifying compound.

In some embodiments, the at least one ABL inhibitor and the at least one mevalonate pathway inhibitor can be administered in conjunction with one or more anti-cancer agents. Examples of anti-cancer agents include, but are not limited to, chemotherapeutic agents (e.g., carboplatin, paclitaxel, pemetrexed, or the like), tyrosine kinase inhibitors (e.g., erlotinib, crizotinib, osimertinib, or the like), immunotherapeutic agents (e.g., pembrolizumab, nivolumab, durvalumab, atezolizumab, or the like), checkpoint inhibitor therapy, antimitotic agents, etc. The at least one ABL inhibitor and the at least one mevalonate pathway inhibitor can also be administered in conjunction with radiotherapy, e.g., external beam radiation; intensity modulated radiation therapy (IMRT), brachytherapy (internal or implant radiation therapy), stereotactic body radiation therapy (SBRT)/stereotactic ablative radiotherapy (SABR), stereotactic radiosurgery (SRS), or a combination of such techniques.

In some instances, the at least one ABL inhibitor and the at least one mevalonate pathway inhibitor can be administered in conjunction with a cholesterol-modifying compound or a pharmaceutical composition thereof. Suitable cholesterol-modifying compounds include, but are not limited to, cholesterol efflux promoters, cholesterol import inhibitors, bile acid sequesterants, and combinations of any thereof.

In other embodiments, the cholesterol-modifying compound may comprise a cholesterol efflux promoter, including but not limited to Liver X Receptor (LXR) agonists. LXR agonists induce the transcriptional activity of LXR target genes, thus attenuate the imbalance of cholesterol metabolism and overactivation of microglia and astrocytes in inflammation and are widely used in a variety of neurodegenerative diseases animal models. Examples include, but are not limited to, T0901317, GW3965 and the like.

In other embodiments, the cholesterol-modifying compound comprises a cholesterol import inhibitor which prevents the uptake of cholesterol by the intestines thereby resulting in the decrease of LDL in the subject. Examples include, but are not limited to, Ezetimibe, Vytorin, and combinations thereof.

In another embodiment, the cholesterol-modifying compound comprises a bile acid sequesterant that binds bile acids thereby lowering LDL-C levels in a subject. Examples include, but are not limited to, cholestyramine resin (Questran), colesevelam (Welchol), colestipol (Colestid), and combinations thereof.

### V. Pharmaceutical Compositions

Pharmaceutical compositions of the ABL inhibitors and the mevalonate pathway inhibitors can take a form suitable for virtually any mode of administration, including, for example, topical, ocular, oral, buccal, systemic, nasal, injection, transdermal, rectal, vaginal, etc., or a form suitable for administration by inhalation or insufflation. Such pharmaceutical compositions typically contain a pharmaceutically acceptable excipient and/or carrier. A “pharmaceutically acceptable excipient and/or carrier” or “diagnostically acceptable excipient and/or carrier” includes but is not limited to, sterile distilled water, saline, phosphate buffered solutions, amino acid-based buffers, or bicarbonate buffered solutions. An excipient selected and the amount of excipient used will depend upon the mode of administration. The ABL inhibitor or mevalonate pathway inhibitor can be formulated in the pharmaceutical composition per se, or in the form of hydrates, solvates, N-oxides, or pharmaceutically acceptable salts. Typically, such salts are more soluble in aqueous solutions than the corresponding free acids and bases, but salts having lower solubility than the corresponding free acids and bases may also be formed.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, CREMOPHORE™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the STING agonist(s) may be formulated as solutions (for retention enemas) suppositories or ointments containing conventional suppository bases such as cocoa butter or other glycerides.

Useful injectable preparations include sterile suspensions, solutions or emulsions of the at least one ABL inhibitor and/or the at least one mevalonate pathway inhibitor in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the at least one ABL inhibitor and/or the at least one mevalonate pathway inhibitor can be dried by any art-known technique, such as lyophilization, and reconstituted prior to use.

For transmucosal administration, penetrants appropriate to the barrier to be permeated are used in the formulation. Such penetrants are known in the art.

For topical administration, the at least one ABL inhibitor and/or the at least one mevalonate pathway inhibitor can be formulated as solutions, gels, ointments, creams, suspensions, etc. as are well-known in the art. Systemic formulations include those designed for administration by injection, e.g., subcutaneous, intravenous, intramuscular, intrathecal, peri-neural, or intraperitoneal injection, as well as those designed for transdermal, transmucosal oral or pulmonary administration. In some embodiments, the STING agonist is administered to a cancer patient via intra-tumoral injection.

Alternatively, other pharmaceutical delivery systems may be employed. Liposomes and emulsions are well-known examples of delivery vehicles that may be used to deliver the at least one ABL inhibitor and/or the at least one mevalonate pathway inhibitor. Certain organic solvents such as dimethyl sulfoxide (DMSO) may also be employed, although usually at the cost of greater toxicity.

For nasal administration or administration by inhalation or insufflation, the STING agonist(s) can be conveniently delivered in the form of an aerosol spray from pressurized packs or a nebulizer with the use of a suitable propellant, e.g., dichlorodifluoromethane, trichlorofluoromethane, dichlorotetrafluoro ethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For prolonged delivery, the at least one ABL inhibitor and/or the at least one mevalonate pathway inhibitor can be formulated as a depot preparation for administration by implantation or intramuscular injection. The at least one ABL inhibitor and/or the at least one mevalonate pathway inhibitor can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the at least one ABL inhibitor and/or the at least one mevalonate pathway inhibitor for percutaneous absorption may be used. To this end, permeation enhancers may be used to facilitate transdermal penetration of the at least one ABL inhibitor and/or the at least one mevalonate pathway inhibitor.

Another aspect of the present disclosure provides all that is described and illustrated herein.

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

It is understood that the examples and embodiments described herein are for illustrative purposes only and that various modifications or changes in light thereof will be suggested to persons skilled in the art and are to be included within the spirit and purview of this application and scope of the appended claims. The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following Examples are provided by way of illustration and not by way of limitation.

Targeting mitochondrial metabolism is emerging as a therapeutic treatment option for cancer patients. The ABL non-receptor tyrosine kinases, ABL1 and ABL2, promote metastasis of lung adenocarcinoma, and enhanced ABL signaling is associated with poor patient prognosis. Unexpectedly, the inventors found that ABL kinases regulate mitochondrial integrity and function and that treatment with ABL allosteric inhibitors decreased oxidative phosphorylation. To identify metabolic vulnerabilities that enhanced this phenotype, the inventors utilized a CRISPR/Cas9 loss-of-function screen targeting 2,322 metabolic enzymes and transporters in the presence of sublethal ABL allosteric inhibitor treatment. HMG-CoA reductase (HMGCR), the rate-limiting enzyme of the mevalonate pathway and target of statin therapies, was identified as a top-scoring sensitizer. Combination treatment of lung cancer cells with sublethal doses of ABL allosteric inhibitors and statins decreased cell survival in a synergistic manner not observed upon treatment with conventional targeted therapies or chemotherapy. Notably, co-treatment of the ABL allosteric inhibitor ABL001 and simvastatin in mouse models of lung cancer brain metastasis and therapy-resistance showed a marked decrease in metastatic burden and concomitant increase in mouse overall survival. This work is also described in Hattaway Luttman, J., et al., Cell Reports 37:109880, Oct. 26, 2021, which is incorporated herein in its entirety for all purposes.

### Example 1. Materials and Methods

DATA AND CODE AVAILABILITY: The CRISPR dataset and corresponding analysis code generated during this study are available at BioProject accession PRINA679091 and https://gitlab.oit.duke.edu/dcibioinformatics/pubs/pendergast-crispr-barcode.

EXPERIMENTAL MODEL AND SUBJECT DETAILS: Cell lines and Cell Culture. PC9 parental cells were a gift from Dr. Joan Massague (Memorial Sloan-Kettering Cancer Center, New York, NY, USA) (Valiente et al., 2014). PC9-GR4 (gefitinib-resistant) cells were a gift from Dr. Passi Jänne (Dana-Farber Cancer Institute, Boston, MA, USA) (Cortot et al., 2013). Large cell carcinoma (LCC) H460 cells were provided by Dr. Fernando Lecanda (University of Navarra, Pamplona, Spain) (Vicent et al., 2008). PC9-BrM3 cell lines were derived in the Pendergast laboratory by serial intracardiac injection as previously described. Human H358 lung cancer cells were purchased from ATCC. Parental and derivative cell line pairs were subjected to short tandem repeat (STR) profiling through the Duke University DNA Analysis Facility Human cell line authentication (CLA) service to confirm their authenticity. Lung cancer cells were maintained in RPMI 1640 (Life Technologies) supplemented with 10% tetracycline-screened fetal bovine serum (FBS, Hyclone), 10 mM HEPES, 1 mM sodium pyruvate, and 0.2% glucose. H293T cells used for transfection and virus production were purchased from ATCC and were maintained in DMEM (Life Technologies) with 10% FBS (Corning). All cultures were maintained at 37° C. in humidified air containing 5% CO₂. For experiments assessing effects of pharmacologic inhibitors in vitro (GNF-5, ABL001, Gefitinib, Docetaxel, Simvastatin, Fluvastatin, FTI-277, GGTI-298), drugs were dissolved in DMSO and the final concentration of DMSO in culture media did not exceed 0.1% v/v. Cholesterol was solubilized in 40% (2-hydroxypropyl)-β-cyclodextrin at room temperature, sterile filtered (0.45 μM) and stored at −20° C. MVA was resolved with 0.1M NaOH, followed by neutralizing with 0.1M HCL/1M HEPES. The ABL allosteric inhibitors GNF-5 and ABL001 were synthesized by the Duke University Small Molecule Synthesis Facility and validated by LC-MS and 1H-NMR, as well as cell-based assays. The following drugs used for in vitro analysis were purchased from: Cayman: Simvastatin (10010344); Sigma: Gefitinib (SML1657), Fluvastatin (SML0038), Mevalonolactone (M4667), Cholesterol (C3045), Geranylgeranyl pyrophosphate (G6025), Farensyl pyrophosphate (F6892); Tocris: FTI-277 (2407) and GGTI-298 (2430); LC Laboratories: Docetaxel (D-1000).

SEAHORSE MEASUREMENTS. Basal and maximal oxygen consumption rate and ATP production were measured using a Mito Stress test Kit and XF96 Extracellular Flux Analyzer (Seahorse Bioscience), according to manufacturer's instructions. Cells were plated in XF96 plates at 10,000 cells per well on Day 0. Cells were treated on Day 1 with IC50 doses of GNF5, ABL001, gefitinib, docetaxel, and vehicle control. On Day 2, media was aspirated and replaced with IC50 dose of each drug in XF Assay Medium (Seahorse Bioscience) supplemented with 10 mM glucose, 1 mM pyruvate, and 2 mM glutamine. The plate was incubated in a non-CO₂ incubator at 37° C. for 1 hr to equilibrate. OCR measurements, taken every 6 min, were collected at baseline and after the sequential addition of oligomycin 1 μM (final concentration), FCCP 0.5 μM, and rotenone 0.75 μM+antimycin A 1.5 μM.

MITOSOX STAINING. MitoSOX was purchased from Thermofisher (cat. M36008). 100,000 cells were plated in six-well plates and treated with vehicle or IC50 doses of indicated drugs for 24 hr. Cells were stained with 5 μM MitoSOX resuspended in serum-free RPMI containing associated drug concentration in the dark for 10 mins in a 37° C. 5% CO2 incubator. Cells were washed once with PBS and trypsinized followed by another wash in PBS and resuspended in 500 μL of PBS. The samples were analyzed using flow cytometer BD FACSCanto II. Gating strategy was defined using untreated/unstained cells. Analysis of flow cytometry data was performed with FlowJo v10.

MITOTRACKER STAINING. MitoTracker Red CMXRos was purchased from Thermofisher (cat. M7512). 100,000 cells were plated in six-well plates and treated with vehicle or IC50 doses of GNF5. Cells were stained with 100 nM MitoTracker resuspended in serum-free RPMI containing associated drug concentration in the dark for 30 mins in a 37° C. 5% CO₂ incubator. Cells were washed once with PBS and trypsinized followed by another wash in PBS and resuspended in 500 μL of PBS. The samples were analyzed using flow cytometer BD FACS Canto II. Gating strategy was defined using untreated/unstained cells. Analysis of flow cytometry data was performed with FlowJo v10.

POOLED CRISPR SCREEN. PC9 cells were seeded into 12, six-well plates at 0.25e6 cells/well. A separate plate was also prepared for no puromycin and puromycin controls of non-transduced cells. Cells were transduced at a MOI of 0.2. 24 hours after viral transduction, cells were replated into puromycin-containing media. A sample was collected at 48 hours of puromycin exposure to confirm library coverage in the transduced population. Transduced cells were expanded in puromycin for a total of 10 days prior to drug introduction, at which point the transduced cell population was split into vehicle (DMSO) and GNF5 treatment conditions and maintained for up to two weeks. Cells were treated with 2 μM GNF5 which corresponded to 20-30% loss in cell viability following a 3-day dose response assay. Cells were counted, replated, and drug replenished every day. At any given point during the screen, each replicate was represented by a minimum of 12E6 cells, sufficient to provide 1000×coverage of the library (1000 cells per unique sgRNA). Samples of 25E6 cells were collected upon screen initiation, termination, and at weekly intervals. Following completion of the screens, DNA was extracted (DNeasy Blood & Tissue Kit, QIAGEN) and prepared for sequencing as previously described.

SCREEN ANALYSIS. Deep sequencing was performed on an Illumina Nextseq platform (75 bp, paired-ended) to identify differences in library composition. All sequencing was performed by the Duke University genome sequencing facility. Barcoded reads were mapped to the guide RNA library using bcSeq to obtain the counts for each guide RNA. Determinations of genetic essentiality and drug sensitization/resistance were made by evaluating differential guide compositions between the initial population and subsequent drug-treated and vehicle-treated cells populations. Briefly, the fractional representation (FR) for the guide reads within a sample was normalized to the total reads attributed to that sample. A direct comparison between two samples was represented by the quotient of the respective FRs in the log 2 scale, which is termed the depletion metric (DM). The guide-level DMs for each gene were then collapsed to gene-level scores by taking the average of the top three most depleted constructs resulting in a biased analysis focused on depleted genes. Genes represented by fewer than 5 guides per condition were excluded from analysis. In the 2,322-gene library, 7 genes (representing 0.3% of the total library) were excluded. Genetic essentiality was calculated by considering the depletion/enrichment of the vehicle-treated (DSMO) population over time (DMSO final/DSMO initial). Drug sensitization/resistance was calculated by considering the depletion/enrichment of the drug-treated population relative to the vehicle-treated population (Drug final/DMSO final). All depletion/enrichment effects are reported as log 2 ratios. All analyses were conducted using the R statistical environment (https://www.r-project.org/) along with extension packages from the comprehensive R archive network (CRAN; available at cran.r-project.org/) and the Bioconductor project. The analyses were carried out with adherence to the principles of reproducible analysis using the knitr package for generation of dynamic reports and gitlab for source code management. The code for replicating the statistical analysis was made accessible through a public source code repository, available at gitlab.oit.duke.edu/dcibioinformatics/pubs/pendergast-crispr-barcode.

Because many metabolic genes are known to be essential to cellular viability, determining the effect of cell-essential genetic loss on apoptosis is difficult. To this point, a subset of essential metabolic genes will have lost representation in our screen before the 10-day puromycin selection period is over; our screen does not capture the effect of these genes (which represent a trivial fraction of our library) on apoptosis. The remaining cell-essential genes are captured by the screen. Since our analysis normalizes the effect of gene knockout+drug treatment to gene knockout alone, the interpretation of these genes does not require additional correction, except that they necessarily suffer from reduced resolution.

CELL VIABILITY ASSAY. Cells were seeded in white-walled clear bottom 96-well plates in triplicate at 3,000 cells per well. Each condition was run in triplicate wells each from three independent experiments and measured using CellTiter-Glo reagent (Promega). Plates were read on a Tecan Infinite M1000 Microplate Reader and results were analyzed in GraphPad.

ANNEXIN V STAINING. Annexin V staining was performed to determine the percentage of cells undergoing apoptosis. 100,000 cells were plated in six-well plates and treated with vehicle, 10 μM GNF5, 1 μM Simvastatin, 0.5 μM Fluvastatin or the combination for 24 hr. Upon collection, cells were trypsinized, washed twice with PBS, resuspended in 100 μL 1×Annexin V binding buffer (BD Biosciences) containing 5 μL Annexin V stain conjugated to APC (allophycocyanin) (BD Biosciences). Phosphatidylserine externalization was measured using APC-conjugated Annexin. Following a 15 min incubation at RT, the samples were analyzed using flow cytometer BD FACSCanto II. Gating strategy was defined using untreated/unstained cells. Analysis of flow cytometry data was performed with FlowJo v10.

IMMUNOBLOTTING PROCEDURES. Cells were lysed in RIPA buffer (50 mM Tris-HCl pH 7.4, 150 mM NaCl, 1 mM EDTA, 1% Triton X-100, 1% sodium deoxycholate and 0.1% SDS) containing protease-phosphatase inhibitor cocktail (Cell Signaling). Cell suspensions were rotated at 4C for 15 minutes followed by microcentrifugation to remove cell debris, and protein concentration was quantified using the DC Protein Assay (BioRad). Equal amounts of protein were separated by SDS/PAGE and transferred onto nitrocellulose membranes using the Transblot Turbo Transfer system (Bio-Rad). Membranes were incubated with primary antibody overnight at 4° C., followed by 3 washes in 1×TBST and incubation with corresponding secondary antibody for 1 hr at room temperature. Blots were developed using SuperSignal West PLUS Chemiluminescent Substrate developing solution (Invitrogen) and imaged using either film or a ChemiDoc XRS+imager (Bio-Rad. The following antibodies used for immunoblot analysis were purchased from: Cell Signaling: Phospho-CrkL (Tyr207) (3181L), beta-Tubulin (D2N5G) (15115S), cleaved PARP (5625), total PARP (9542), cleaved caspase 3 (9661), total caspase 3 (9668), cytochrome C (11940), beta-Catenin (8480); Thermofisher: HDJ2 (MA5-12748); Millipore Sigma: ABL1 (8E9) (MAB1130), ABL2 (6D5) (H00000027-M03); Santa Cruz: RAP1 (sc-398755), CRKL (C-20) (sc-319), GAPDH (6C5) (sc-32233); Jackson Immunoresearch: Peroxidase AffiniPure Goat Anti-Mouse IgG (H+L) (115-035-003), Peroxidase AffiniPure Goat Anti-Rabbit IgG (H+L) (115-035-144).

REAL-TIME QUANTITATIVE PCR. RNA was isolated from subconfluent monolayers of cancer cells using the RNeasy RNA isolation kit (QIAGEN), and cDNA synthesis was performed using oligo(dT) primers and M-MLV reverse transcriptase (Invitrogen). RT-qPCR was performed in triplicate wells using iTaq Universal SYBR Green Supermix (Bio-Rad). Primers used in this study were purchased from Sigma Aldrich. Analysis of real-time data was collected using a Bio-Rad CFX384 machine and CFX Maestro software. Expression levels of each gene were normalized to GAPDH control housekeeping genes using the ddCT algorithm. Primers sequences used are listed in Table 2.

TABLE 2
RT-PCR Primers

| Primer | Sequence |
|---|---|
| BBC3 Fwd | GACCTCAACGCACAGTACGAG (SEQ ID NO: 1) |
| BBC3 Rev | AGGAGTCCCATGATGAGATTGT (SEQ ID NO: 2) |
| Bcl2 Fwd | GGTGGGGTCATGTGTGTGG (SEQ ID NO: 3) |
| Bcl2 Rev | CGGTTCAGGTACTCAGTCATCC (SEQ ID NO: 4) |
| Bcl2L1 Fwd | GAGCTGGTGGTTGACTTTCTC (SEQ ID NO: 5) |
| Bcl2L1 Rev | TCCATCTCCGATTCAGTCCCT (SEQ ID NO: 6) |
| GAPDH-Fwd | TGTGGGCATCAATGGATTTGG (SEQ ID NO: 7) |
| GAPDH-Rev | ACACCATGTATTCCGGGTCAAT (SEQ ID NO: 8) |

INTRACARDIAC INJECTIONS. All animal experiments were conducted in accordance with protocols approved by the Duke University Division of Laboratory Animal Resources Institutional Animal Care and Use Committee (IACUC). Cells were stably transduced with pFU-luciferase-Tomato (pFuLT) DNA prior to injection to allow for bioluminescent imaging (BLI) in vivo. 8-12-week old age-matched female athymic nu/nu mice were used for all studies (Jackson Laboratory). Mice were anesthetized with 5% isoflurane prior to injections. For all studies, 4×105 lung cancer cells suspended in 100 μL PBS were injected into the left cardiac ventricle with a 30-gauge needle. Animals were monitored until full recovery from anesthesia and were subsequently imaged weekly to both confirm proper anatomical injection and to monitor for progression of disease burden using an IVIS XR bioluminescent imager. The ABL allosteric inhibitor ABL001 (Asciminib) was used for in vivo inhibition of the ABL kinasesin tumor-bearing mice and was prepared as a suspension in sterile 0.5% methylcellulose/0.5% Tween-80 as described previously (Wylie et al., 2017). Mice were treated with either vehicle control or 100 mg/kg QD (daily) ABL001 via oral gavage once per day. ABL001 was synthesized by the Duke University Small Molecule Synthesis Facility and validated by LC-MS and 1H-NMR. Simvastatin was purchased from Toronto Research Chemicals (cat. S485000) and dissolved in aqueous 2% dimethylsulfoxide (DMSO), 30% polyethylene glycol 400 (PEG 400), and 5% Tween 80. Mice were treated with either vehicle control or 10 mg/kg/QD simvastatin. To account for potential interactions between the two drugs and solvents, mice were treated each morning with either simvastatin or vehicle control, and two hours later with either ABL001 or vehicle control via oral gavage. The presence of brain metastases was confirmed through in vivo BLI followed by isolation of brains for OCT or paraffin sectioning. Living Image software was used for analysis of BLI data.

IMMUNOFLUORESCENCE AND CONFOCAL MICROSCOPY. Brains were perfused and fixed with 4% paraformaldehyde in PBS prior to extraction. Upon extraction, brains were rotated overnight (0/N) in 4% paraformaldehyde in PBS at 4° C. followed by subsequent washes in PBS the following day. For OCT embedding, brains underwent sucrose protection in 15% sucrose in water at 4° C. O/N rotation following by 0/N rotation in 30% sucrose in water at 4° C. before OCT embedding at −80° C. OCT sections were 10 μm thick. For paraffin embedding, brains were placed into 70% ethanol prior to paraffin embedding. Paraffin embedding was performed at the Duke University Immunohistopathology Core Facility sections were cut at 5 μm thick. OCT sections were thawed at room temperature for 15 minutes followed by acetone fixation. Paraffin sections were deparaffinized, rehydrated, and heat inactivated (BioCare Medical Decloaking Chamber). Both deparaffinized and OCT sections were then washed in PBS and blocked in 3% goat serum in PBS with 0.05% Tween-20 for one hour. Sections were incubated with primary antibodies in blocking solution overnight at 4° C. in a humidified chamber at concentrations indicated below. Sections were then washed with PBS followed by incubation with the appropriate secondary antibody in blocking solution for one hour at room temperature. Sections were then washed with PBS, incubated with the nuclear stain, Hoechst33342, and washed again with PBS before mounting using aqueous mounting media (Dako-53025). Antibodies for immunofluorescence and IHC experiments included: cleaved caspase 3 (Cell Signaling 9661) at 1:100 dilution, Ki67 (Cell Signaling 9449) at 1:200 dilution, tdTomato (Kerafast EST203) at 1:100 dilution. All images were captured on an Axio Imager D10 (Carl Zeiss) with a 20×/0.75 EC Plan-Neofluar objective lens.

DNA PLASMIDS. The sequences for shRNAs targeting the ABL kinases are listed in Table 2. Stable non-inducible shRNAs against non-target control (NTC) and HMGCR in the pLK0.1 vector were from the Sigma Mission TRC1 Lentiviral snRNA library and were obtained through the Duke Functional Genomics Shared Resource Facility. Sequences and Sigma clone identifiers for each of these shRNAs are listed in Table 3.

TABLE 3
ShRNA Sequences

| shRNA | Sequence |
|---|---|
| scrambled | GGTGTATGGGCTACTATAGAA (SEQ ID NO: 9) |
| shRNA | |
| ABLI shRNA | GGTGTATGAGCTGCTAGAGAA (SEQ ID NO: 10) |
| ABL2 shRNA | CCTTATCTCACCCACTCTGAA (SEQ ID NO: 11) |
| HMGCR shRNA | CCGGGCAGTGATAAAGGAGGCATTTCTCGAGAAA |
| 46448 | TGCCTCCTTTATCACTGCTTTTTG (SEQ ID NO: |
| | 12) |
| HMGCR shRNA | CCGGCCTGCTGCTATAAATTGGATACTCGAGTATC |
| 46452 | CAATTTATAGCAGCAGGTTTTTG (SEQ ID NO: |
| | 13) |

QUANTIFICATION AND STATISTICAL ANALYSES. Statistical analyses were performed using GraphPad Prism 7 and GraphPad Prism 9 software. Mouse numbers per group were determined through statistical power calculations where 10 mice per group allows for 90% power, at the unadjusted 0.05 two-sided level, to detect inter-group differences of 50% and assuming intra-group differences of 25%. For Kaplan-Meier survival curves, p values were calculated using log-rank (Mantel-Cox) testing. P values below an adjusted p<0.017 were deemed significant accounting for 3 pairwise comparisons. Statistical analysis of tumor flux was evaluated by ANOVA followed by Fisher post hoc testing to calculate p values and those less than 0.05 were quantified as statistically significant. For comparisons between mouse groups of unequal size, the mean value and SEM were used to allow for statistical analysis by ANOVA. Bar graph data represent averages±SEM.

STUDY APPROVAL. All procedures involving mice were approved and performed following the guidelines of the IACUC of Duke University Division of Laboratory Animal Resources.

### Example 2. ABL Kinase Allosteric Inhibitors Regulate Mitochondria Function in Lung Cancer Cells

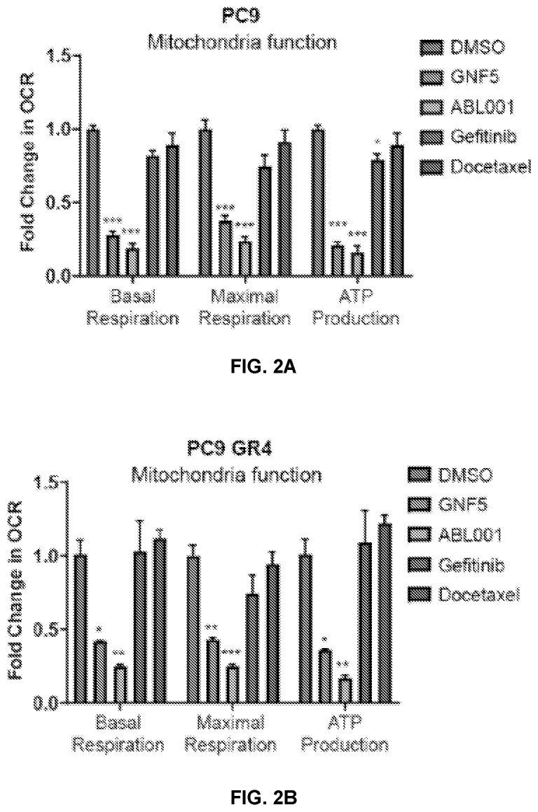

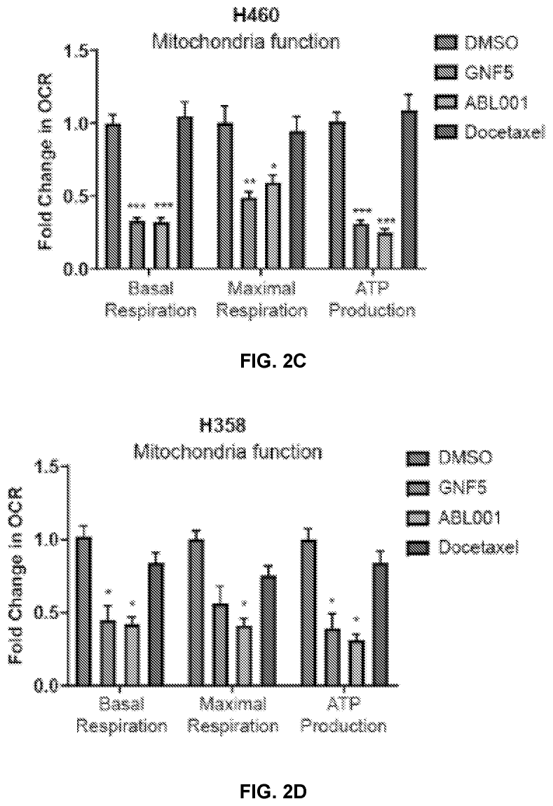

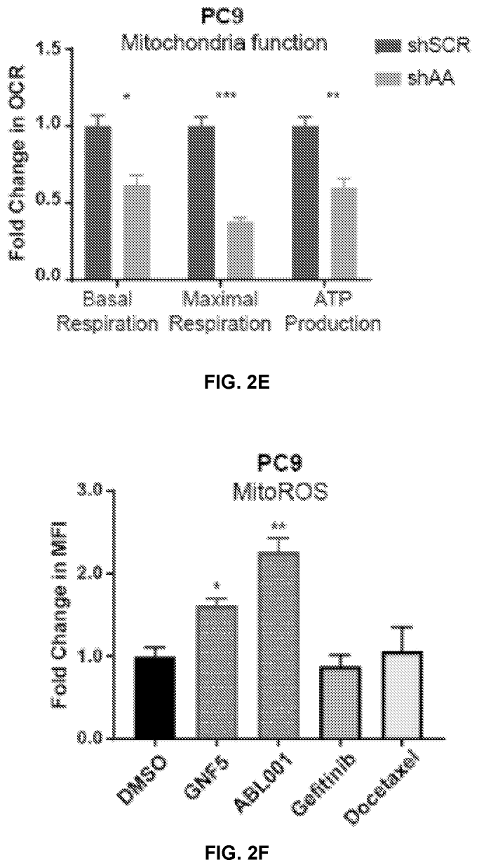

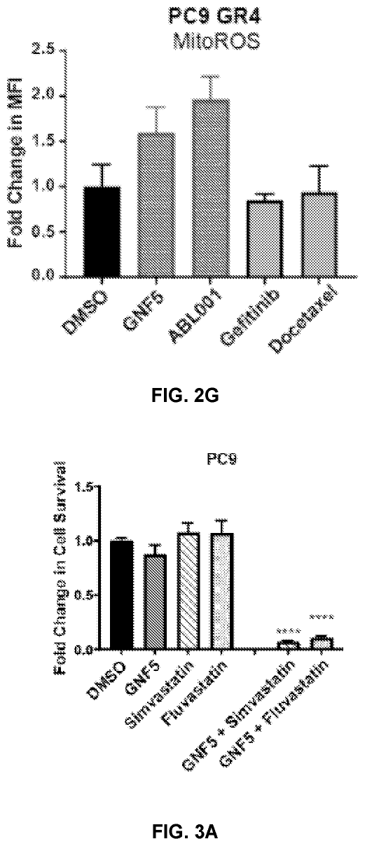

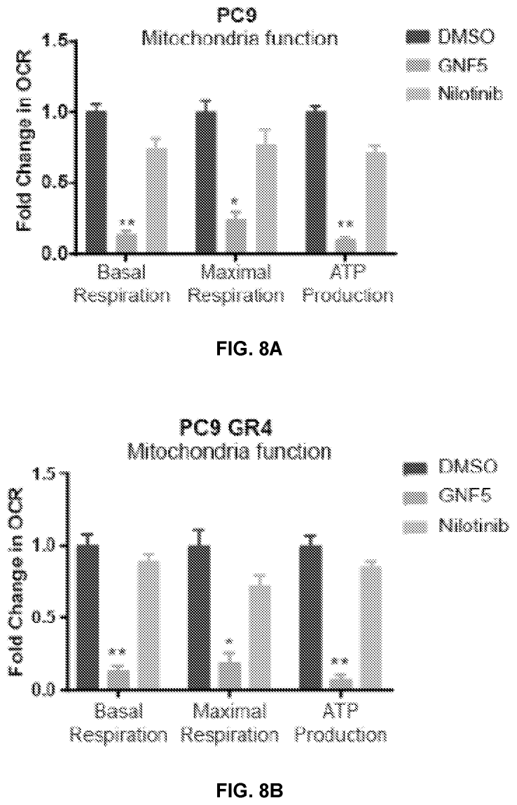

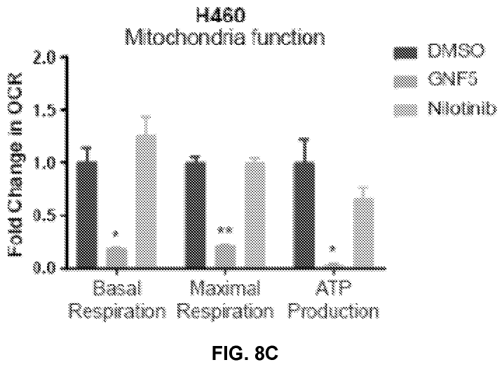

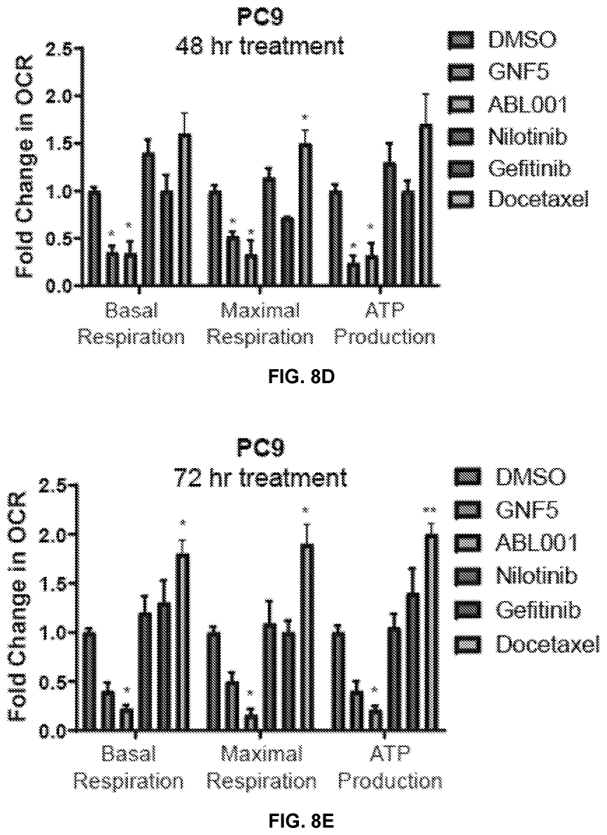

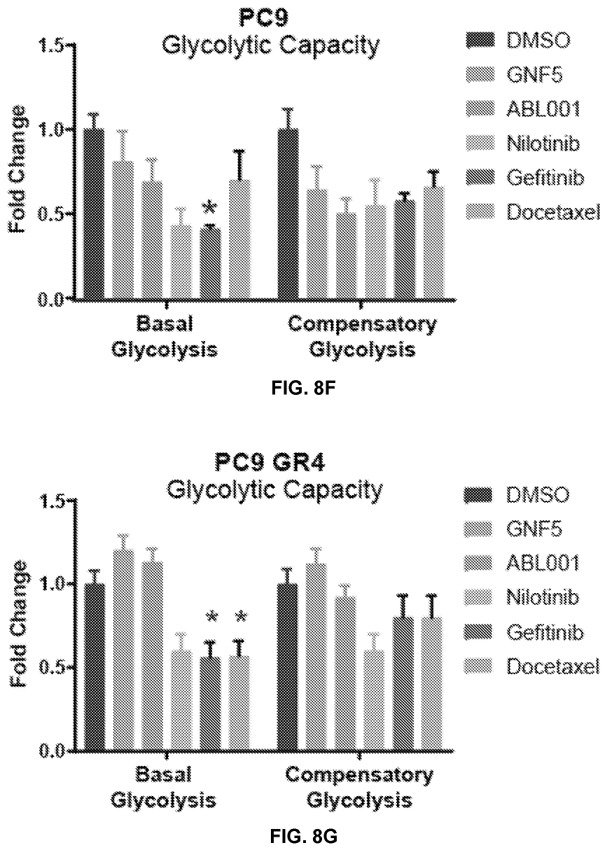

It was investigated whether inhibition of the ABL kinases could perturb mitochondrial function in lung adenocarcinoma cells with oncogenic mutations in EGFR, either sensitive to EGFR tyrosine kinase inhibitors (TKIs) (PC9) or TKI-resistant (PC9 GR4), as well as KRAS mutant large cell lung carcinoma (LCC) H460 cells and KRAS mutant lung adenocarcinoma H358 cells. Lung cancer cells were analyzed by Seahorse XF Analyzer Mito Stress Test for mitochondrial basal respiration, maximal respiration, and ATP production as measured by changes in oxygen consumption rate (OCR) following treatment with ABL kinase inhibitors (FIGS. 2A-2D). For these studies we used ABL allosteric inhibitors, GNF5 and ABL001 (Asciminib), which bind with high affinity to the unique myristate-binding pocket of the ABL kinases, as well as the second-generation ABL ATP-competitive inhibitor Nilotinib. Surprisingly, only the ABL allosteric inhibitors markedly impaired mitochondria basal respiration, maximal respiration, and ATP production (FIGS. 2A-2D; FIGS. 8A-8C). To validate the results of the screen, PC9 cells were treated with sublethal doses of GNF5 and two statins, simvastatin and fluvastatin. The inability of Nilotinib to inhibit mitochondria function might be due to the lack of specificity of ABL ATP-competitive inhibitors as these drugs inhibit multiple enzymes other than ABL in solid tumors, and/or the inability of Nilotinib to disrupt interactions with specific downstream targets, which was shown can be blocked by ABL allosteric inhibitors through binding to a distinct site in the ABL kinase domain. Moreover, treatment with ABL ATP-competitive inhibitors, but not allosteric inhibitors, induces activation of the RAF-ERK pathway in diverse cancer cell types. Notably, genetic knockdown of ABL1 and ABL2 (shAA) demonstrated that depletion of the ABL kinases similarly decreased mitochondrial respiration and ATP production in EGFR and KRAS mutant lung cancer cells (FIG. 2E; FIG. 8A).

It was also evaluated whether aberrant mitochondria function induced by treatment with ABL allosteric inhibitors was observed following treatment with two current FDA-approved therapeutics for lung adenocarcinoma patients: gefitinib, an EGFR TKI, and docetaxel, a taxane chemotherapy. Lung cancer cells were treated with IC50 drug doses determined by dose-response assays (Table 4). Strikingly, mitochondrial function as measured by basal and maximal respiration, and ATP production, was greatly decreased following treatment with either GNF5 or ABL001 in lung cancer cells harboring EGFR or KRAS mutations (FIGS. 2A-2D), but was not impaired upon treatment with gefitinib in EGFR mutant PC9 parental cells and gefitinib-resistant PC9 GR4 cells (FIGS. 2A-2B). Further, treatment with docetaxel in the EGFR mutant cells, as well as KRAS mutant H460 and H358 lung cancer cells, did not significantly impair mitochondria function (FIGS. 2C-2D). These findings show that ABL allosteric inhibitors preferentially impair mitochondrial function in lung cancer cells with diverse oncogenic drivers.

TABLE 4
IC50 Calculations for Cell Lines

| Cell Line | Drug | Calculated IC50 | |
|---|---|---|---|
| PC9 | GNF5 | 10 | μM |
| | ABL001 | 7.5 | μM |
| | Gefitinib | 15 | nM |
| | Docetaxel | 1 | nM |
| | Nilotinib | 2 | μM |
| | Simvastatin | 4 | μM |
| | Fluvastatin | 2 | μM |
| | FTI-277 | 50 | μM |
| | GGTI-298 | 20 | μM |
| PC9 GR4 | GNF5 | 10 | μM |
| | ABL001 | 7.5 | μM |
| | Gefitinib | 200 | nM |
| | Docetaxel | 1 | nM |
| | Nilotinib | 2 | μM |
| | Simvastatin | 4 | μM |
| | Fluvastatin | 2 | μM |
| | FTI-277 | 50 | μM |
| | GGTI-298 | 20 | μM |
| H460 | GNF5 | 15 | μM |
| | ABL001 | 15 | μM |
| | Docetaxel | 0.5 | nM |
| | Nilotinib | 2 | μM |
| | Simvastatin | 12 | μM |
| | Fluvastatin | 4 | μM |
| | FTI-277 | 15 | μM |
| | GGTI-298 | 15 | μM |
| H358 | GNF5 | 15 | μM |
| | ABL001 | 15 | μM |
| | Docetaxel | 1 | nM |
| | Simvastatin | 12 | μM |
| | Fluvastatin | 4 | μM |
| PC9 BrM3 | GNF5 | 10 | μM |
| | ABL001 | 7.5 | μM |
| | Gefitinib | 15 | nM |
| | Docetaxel | 1 | nM |
| | Simvastatin | 4 | μM |
| | Fluvastatin | 2 | μM |

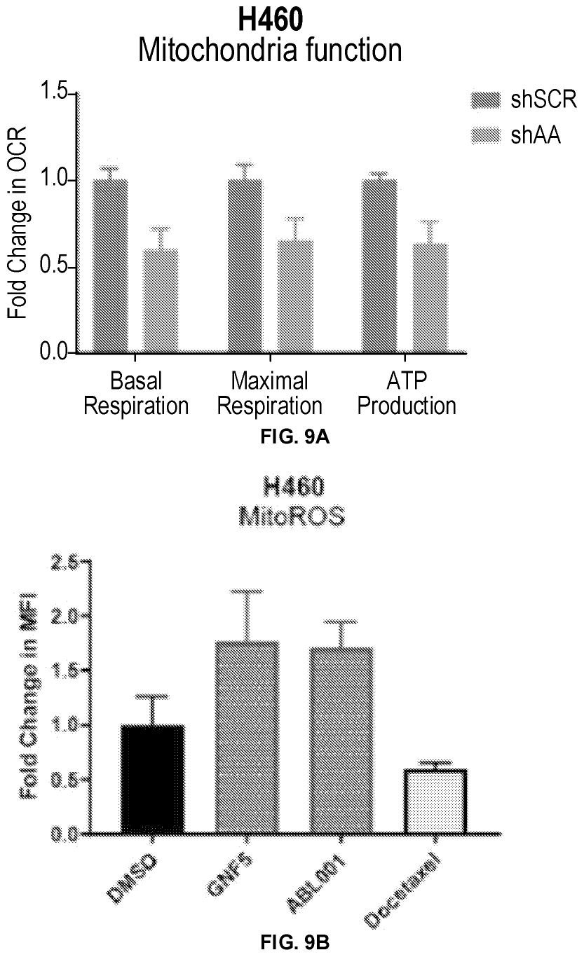

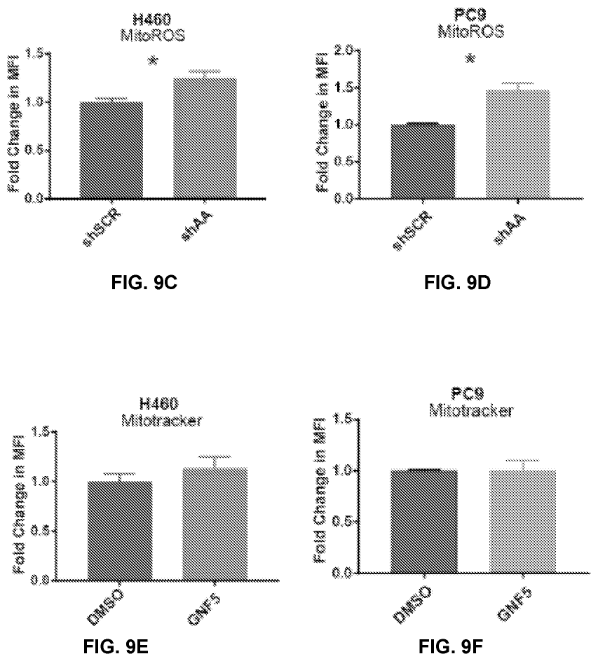

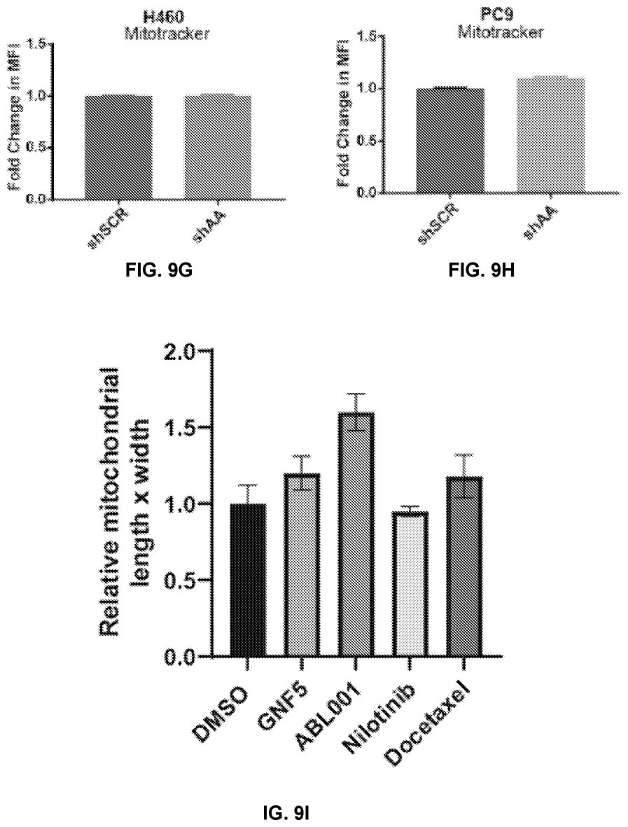

To dissect the mechanism by which mitochondria function is impaired by ABL allosteric inhibitors, mitochondrial superoxide release was examined to identify changes in organelle integrity. It was observed that mitochondrial reactive oxygen species (MitoROS) levels were increased upon GNF5 or ABL001 treatment, but not following gefitinib or docetaxel treatment in EGFR mutant lung cancer cells sensitive or resistant to gefitinib therapy (FIGS. 2F-2G). MitoROS levels were also increased in KRAS mutant H460 cells upon ABL allosteric inhibitor treatment, but not by docetaxel (FIG. 9B). Thus, ABL allosteric inhibitors impair organelle integrity in lung cancer cells irrespective of oncogenic driver (FIG. 9B). MitoROS levels were also increased following ABL1 and ABL2 knockdown in PC9 and H460 cells (FIGS. 9C-9D). Analysis of mitochondria number following knockdown or pharmacologic inhibition of the ABL kinases did not show detectable changes in mitochondria numbers in PC9 and H460 cells indicating that the decrease in OCR is not due to changes in mitochondrial density but rather mitochondria function (FIGS. 9E-9H). Examination of changes in mitochondrial morphology by immunofluorescence staining with MitoTracker revealed that ABL001 caused a marginal, nonsignificant increase in mitochondrial network morphology, while the other drugs did not affect mitochondria length and width (FIG. 9I).

Metabolically focused CRISPR/Cas9 loss-of-function screen identifies HMGCR inhibition with statin therapy as an apoptotic sensitizer in lung cancer cells. Because inhibition of ABL kinases impairs oxidative mitochondrial metabolism, it was sought to determine whether targeting additional metabolic nodes enhanced sensitivity to ABL inhibition. Thus, a CRISPR/Cas9 loss-of-function screen targeting 2,322 metabolic enzymes and transporters in the absence and presence of sublethal doses of the ABL allosteric inhibitor GNF5 that corresponded to a 20% loss in cell viability following a 3-day dose response assay was employed. Library-transduced cells were puromycin selected and grown for 10 days prior to treatment. The cells were then exposed to either vehicle or GNF5 for two weeks after which DNA was extracted from cell samples and polymerase chain reaction (PCR) was used to amplify and index barcode short guide RNA (sgRNA) amplicons, and the composition of sgRNA pools was deconvoluted through deep sequencing. The screen was validated for known essential genes by comparing the final and initial sgRNA pools in the vehicle treated screen as previously described. Depletion metrics for each sgRNA were determined by normalizing the relative abundance of each construct following GNF5 treatment to the construct quantity present in vehicle treated cells. The three most depleted constructs per gene were averaged to produce a gene-level three score as previously detailed (Table 5) [end of Detailed Description]. TS scores were ranked allowing for identification of genes that were specifically depleted or enriched in the GNF5 treated cell population (data not shown). The subset of depleted genes that fell below the inflection point of the curve to experimentally was focused on to evaluate whether loss of the top 5% of deleted genes could potentiate the cell killing effects of ABL allosteric inhibition (Table 5). Among these hits were metabolic enzymes and transporters that converged on metabolic nodes that regulate cholesterol synthesis and mobilization, as well as complexes of the electron transport chain. Focus was made on targets that could be pharmacologically inhibited with FDA-approved drugs. HMG-CoA reductase (HMGCR), the rate-limiting enzyme of the mevalonate pathway, was identified as a top-scoring reactive sensitizer to cell death in the presence of low dose GNF5 (data not shown). HMGCR was selected for further study because it was in the top 1% of depleted genes and is the target of statin therapies commonly prescribed for patients with high cholesterol. Statins have a highly tolerable pharmacokinetic profile and availability making HMGCR an attractive target for combination therapy.

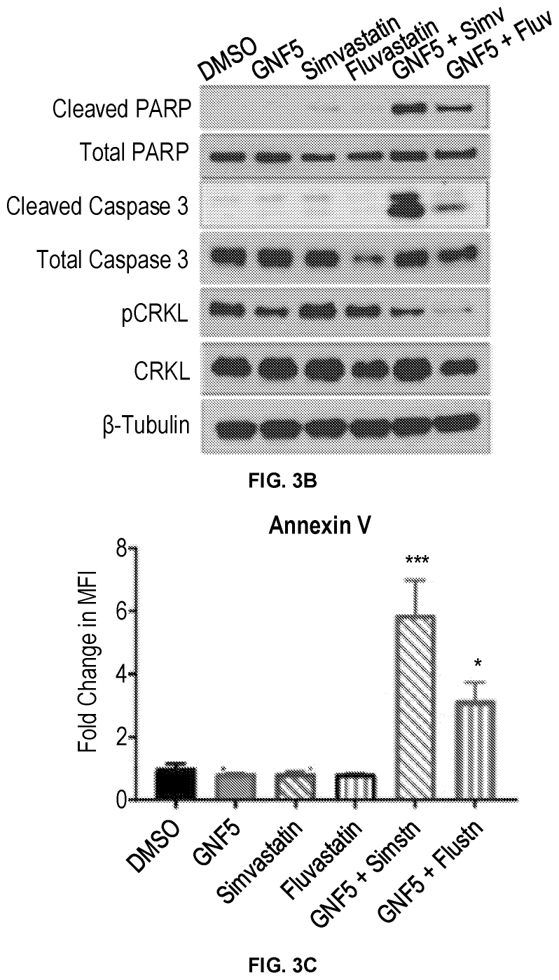

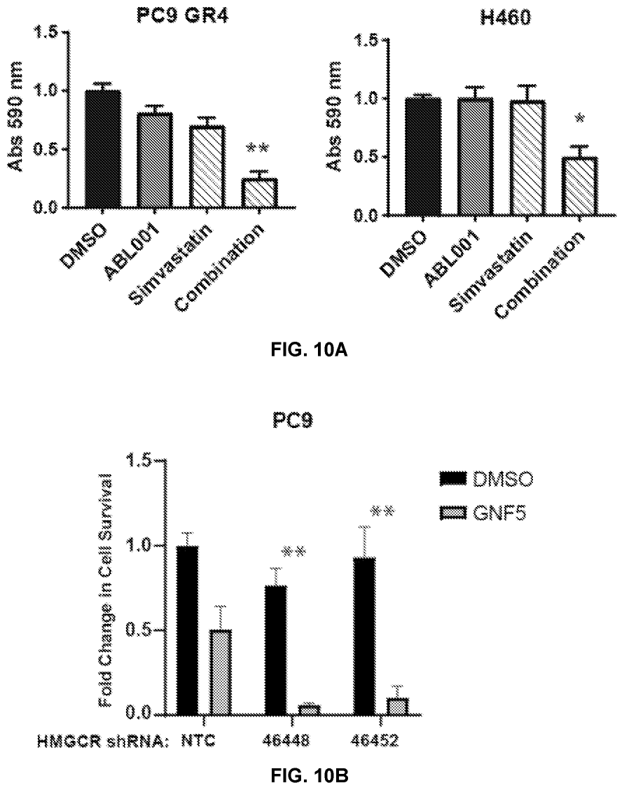

To validate the results of the screen, PC9 cells were treated with sublethal doses of GNF5 and two statins, simvastatin and fluvastatin. Following 72 hr of combination treatment, over 90% of cells underwent cell death (FIG. 3D). Similar results were seen when cells were cultured in colony formation assays (FIG. 10A). To validate the on-target effect of statins, shRNAs against HMGCR were transduced into PC9 lung cancer cells, and these cells were then treated with GNF5 (FIG. 10A). Immunoblotting for HMGCR and β-Tubulin shows knockdown of HMGCR protein with both shRNAs #46448 and #46452 (data not shown). Markedly decreased cell survival in HMGCR-knockdown was found compared to control lung cancer cells following treatment with GNF5 (FIGS. 10A-10B). Further, immunoblotting revealed dramatic induction of the apoptotic mediators cleaved poly (ADP-ribose) polymerase (PARP) and cleaved caspase-3 following 24 hr of combination treatment with GNF5 and statins, which was consistent with identification of apoptotic cells by flow cytometry of annexin V-stained lung cancer cells co-treated with GNF5 and statins (FIGS. 3E-3F). These data reveal a novel treatment paradigm whereby HMGCR inhibition combined with ABL allosteric inhibition sensitizes cells towards apoptotic cell death.

### Example 3. ABL Allosteric Inhibitors Preferentially Synergize with Statins to Induce Lung Cancer Cell Death

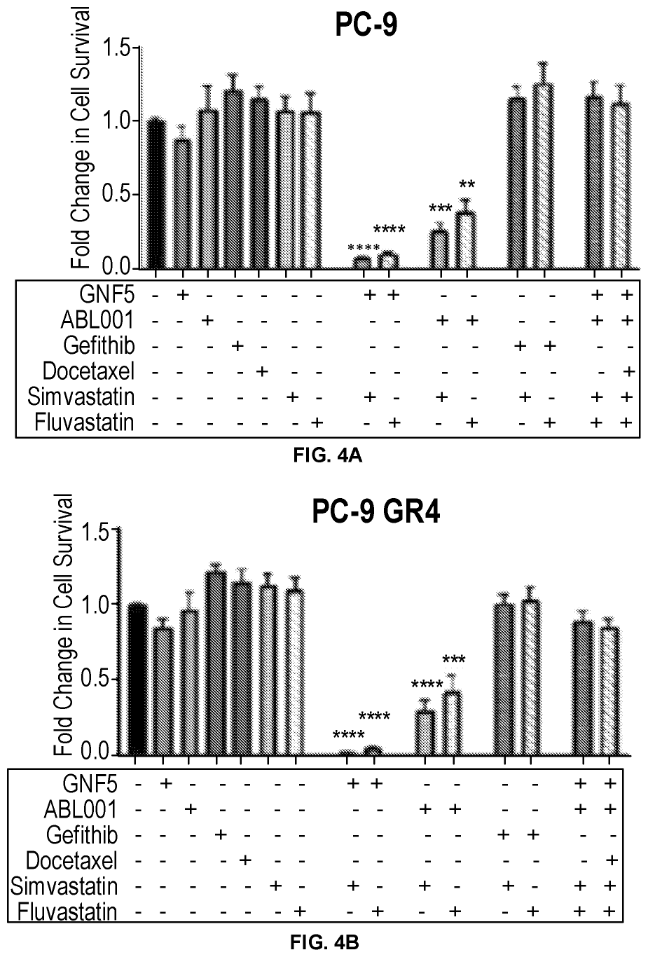

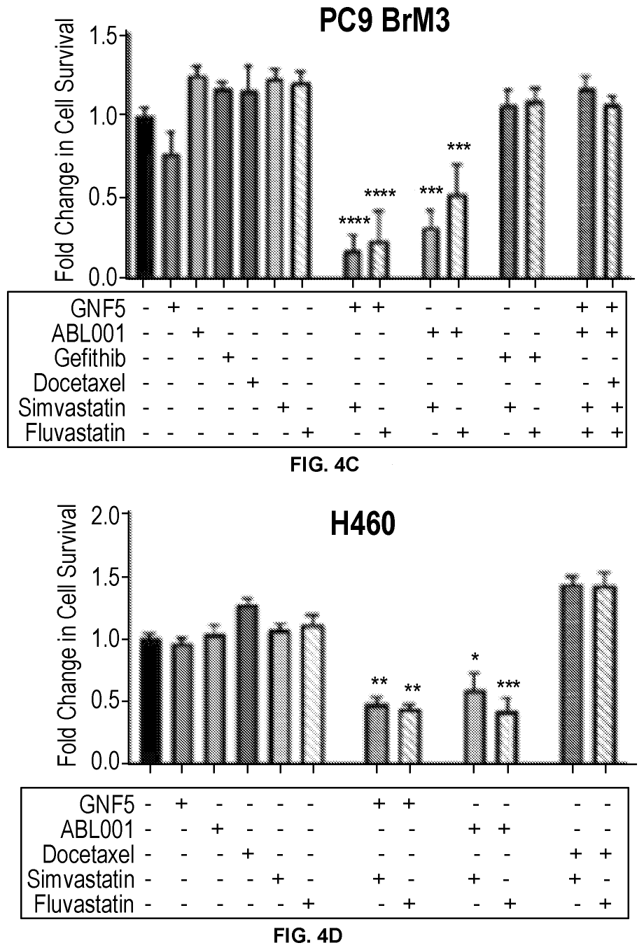

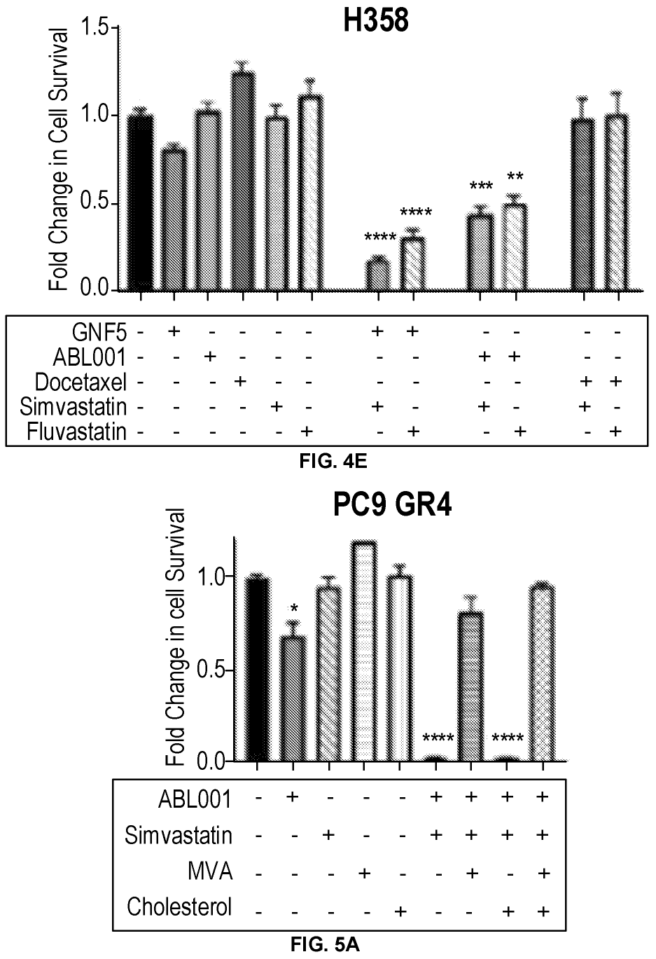

To assess whether ABL allosteric inhibitors preferentially synergize with statins, lung cancer cells were treated at equivalent sublethal doses below the IC₅₀ value of each drug as determined with dose response assays for each cell line. Notably, only the ABL allosteric inhibitors exhibited enhanced cell killing effects upon combination with either simvastatin or fluvastatin in EGFR mutant cells sensitive to EGFR TKIs (PC9), resistant to gefitinib (PC9 GR4), or harboring metastatic tropism to the brain (PC9 BrM3) (FIGS. 4A-4C). Similar findings were observed in KRAS mutant H460 and H358 cancer cells lines (FIGS. 4D-4E). The combination of ABL allosteric inhibitors with statins was found to be synergistic across cell lines as assessed by the Bliss formula for synergy, where a score of 1 indicates true synergy (data not shown).

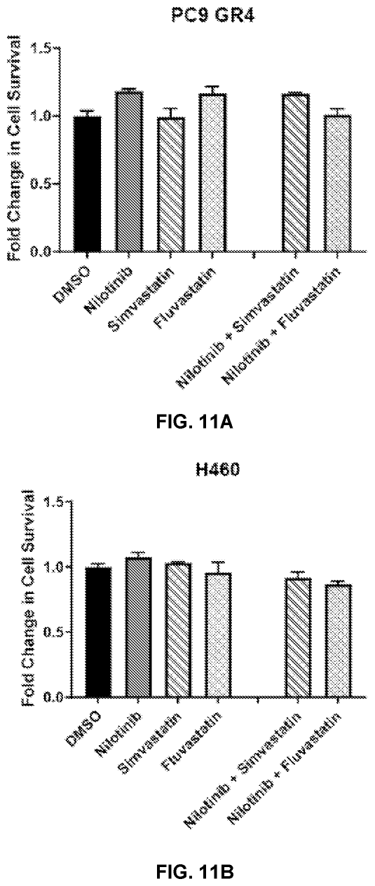

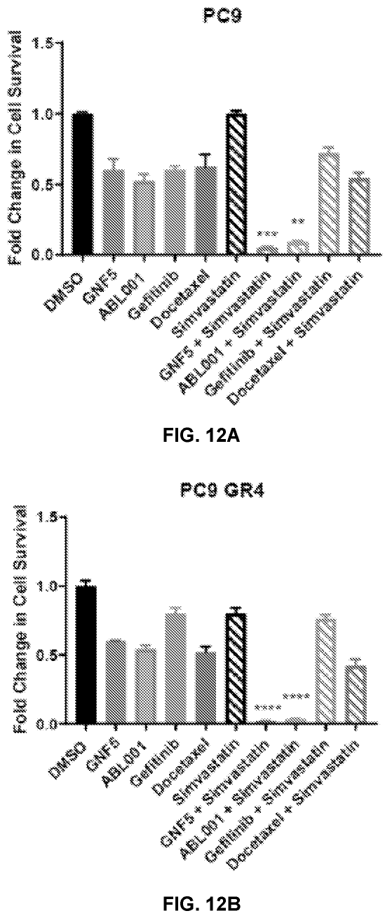

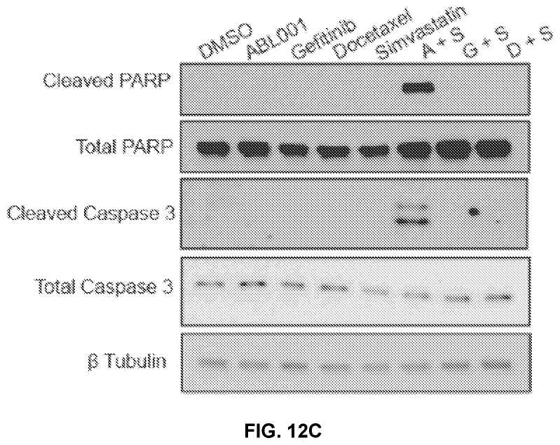

Next, it was sought to evaluate whether sensitization to statin treatment was specific to the ABL allosteric inhibitors or could also be induced by ABL ATP-site inhibitors. Co-treatment of PC9 GR4 and H460 cells with sublethal doses of Nilotinib and either simvastatin or fluvastatin did not promote additive or synergistic decreases in cell viability (FIG. 11A). There are conflicting in vitro reports indicating additive cell killing effects of chemotherapy with high-dose statin therapeutics. Thus, lung cancer cells were treated with IC₅₀ doses of GNF5, ABL001, gefitinib, and docetaxel to determine whether treatment at higher than sublethal doses could enhance the cell killing effects of these drugs. Again, it was found that only ABL allosteric inhibitors could synergize with simvastatin and promote cleavage of PARP and caspase-3 in PC9 cells (FIGS. 12A-12C). In this regard, clinical trial data have shown that statins in combination with various chemotherapies had either marginal or no effect on progression-free survival or overall survival in lung cancer patients. Together, these results show that in contrast to gefitinib and docetaxel, ABL allosteric inhibitors are preferentially capable of sensitizing lung cancer cells to statins and dramatically inhibit cancer cell survival.

### Example 4. Mevalonate, but not Cholesterol, Rescues Cell Survival in Lung Cancer Cells Co-Treated with Statins and ABL Allosteric Inhibitors

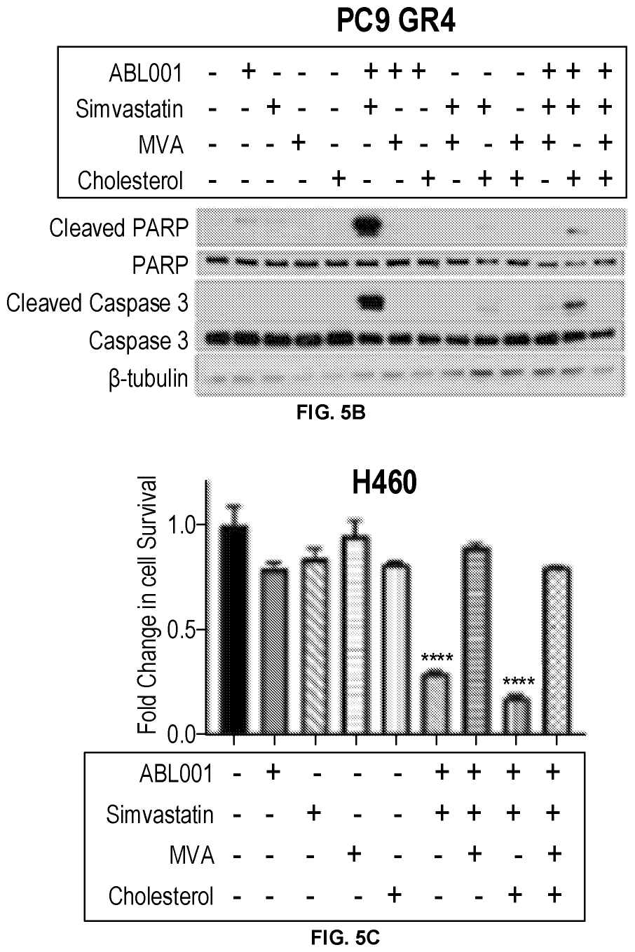

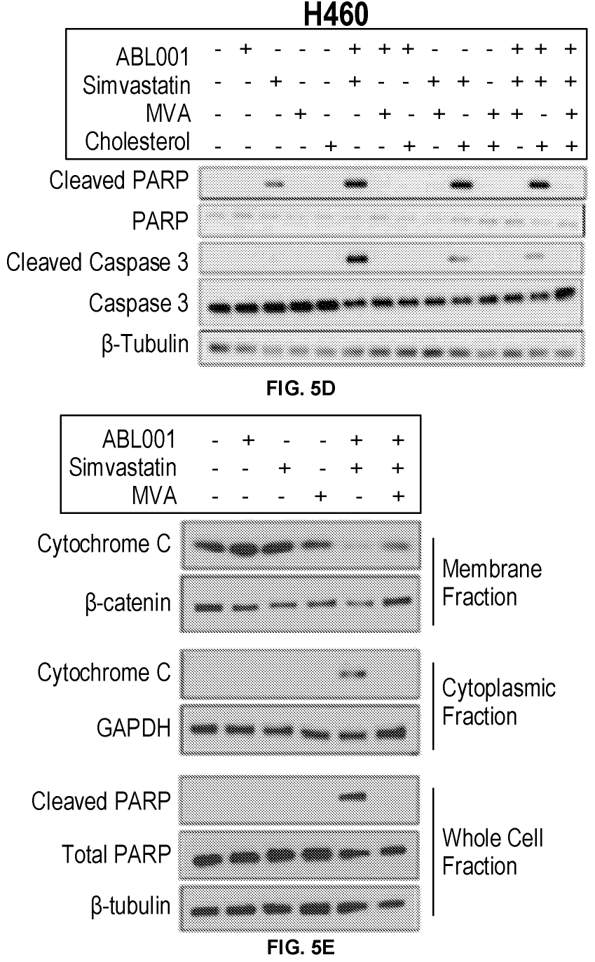

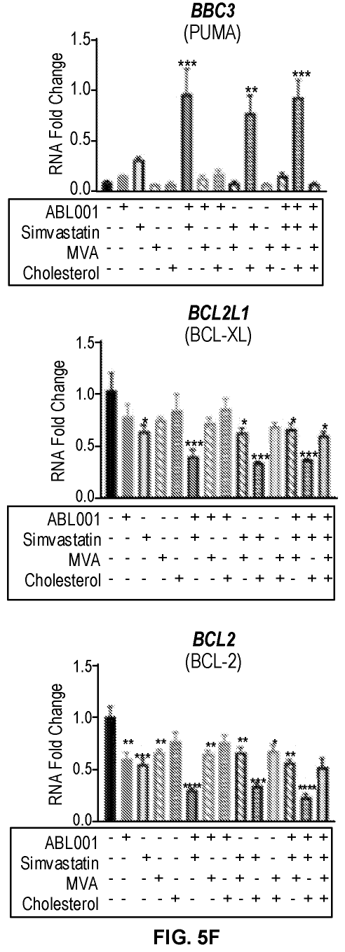

The mevalonate (MVA) pathway catalyzes the conversion of acetyl-CoA to HMG-CoA which is then converted by HMGCR into mevalonate (FIG. 3C). Mevalonate is required for the generation of cholesterol and isoprenoids among other end products. Rescue experiments were performed to identify if MVA or the downstream metabolite cholesterol could reverse the cell killing effect induced by low dose simvastatin treatment in cells co-treated with sublethal doses of ABL allosteric inhibitors. Interestingly, only MVA, but not cholesterol, could rescue cell survival in PC9 GR4 and H460 lung cancer cells (FIGS. 5A and 5C). Consistent with these findings, addition of MVA, but not cholesterol, prevented cleavage of PARP and caspase-3 suggesting that sensitization of lung cancer cells to apoptosis induced by ABL allosteric inhibitors in combination with statin therapy is independent of cholesterol (FIGS. 5B and 5D).

The apoptotic cascade is mediated by interplay among BCL-2 family proteins comprised of pro-apoptotic and anti-apoptotic proteins. Following combination treatment with ABL allosteric inhibitors and statins, we observed that gene expression of the pro-survival factors BCL-2 and BCL-XL was downregulated, while expression of pro-apoptotic PUMA was increased (FIG. 5E). Supplementation with MVA, but not cholesterol, restored gene expression back to baseline (FIG. 5F). Changes in BCL-2 family member expression can elicit pore formation in the mitochondria, resulting in mitochondria outer membrane permeabilization, leading to cytochrome c release. Subcellular fractionation revealed release of cytochrome c from the mitochondrial membrane fraction into the cytosol in response to co-treatment with ABL001 and Simvastatin, which was reversed following the addition of MVA (FIG. 5E). These data show that ABL001 and Simvastatin combination therapy alters gene expression of BCL-2 family members leading to permeabilization of the mitochondria, release of cytochrome c into the cytosol, and cleavage of caspase 3, and that these processes can be reversed by the addition of MVA.

### Example 5. Apoptotic Sensitization to Statin Therapy by ABL Allosteric Inhibitors Requires Inhibition of Protein Prenylation

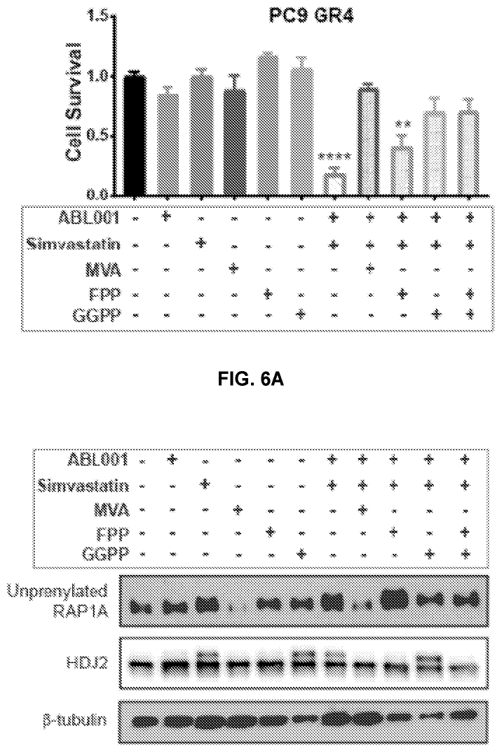

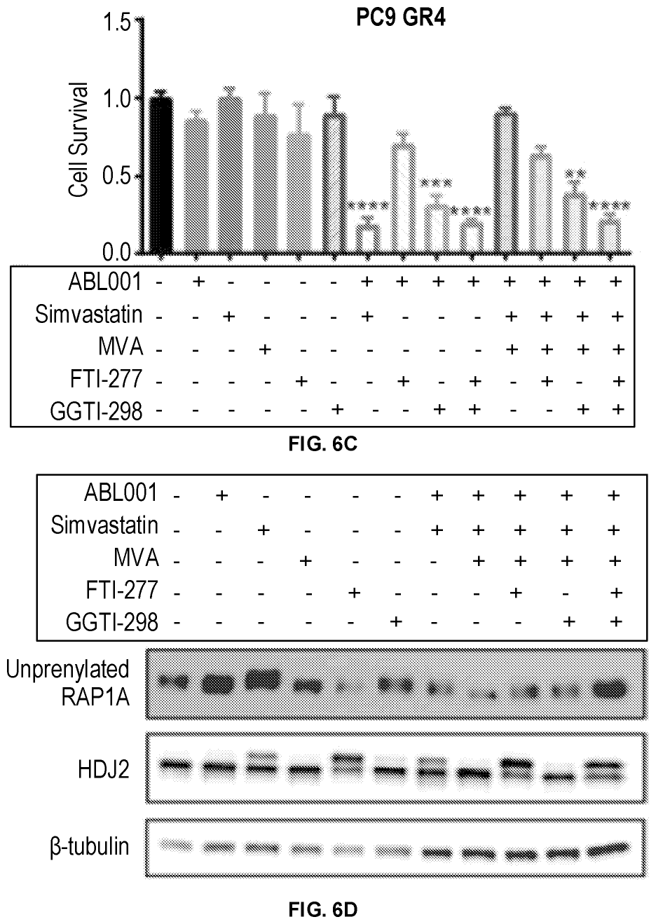

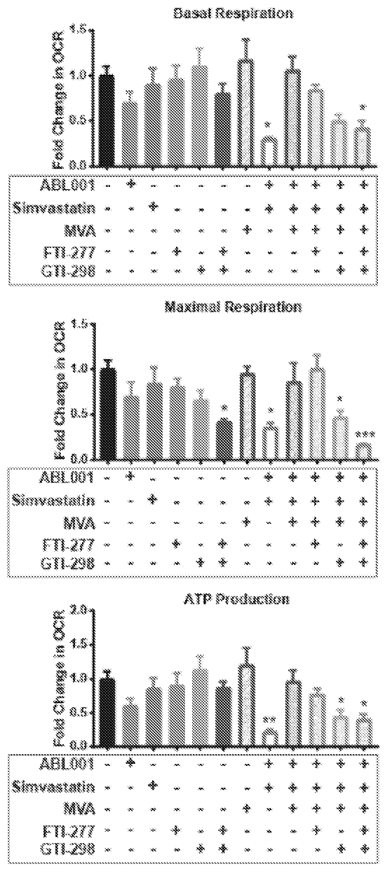

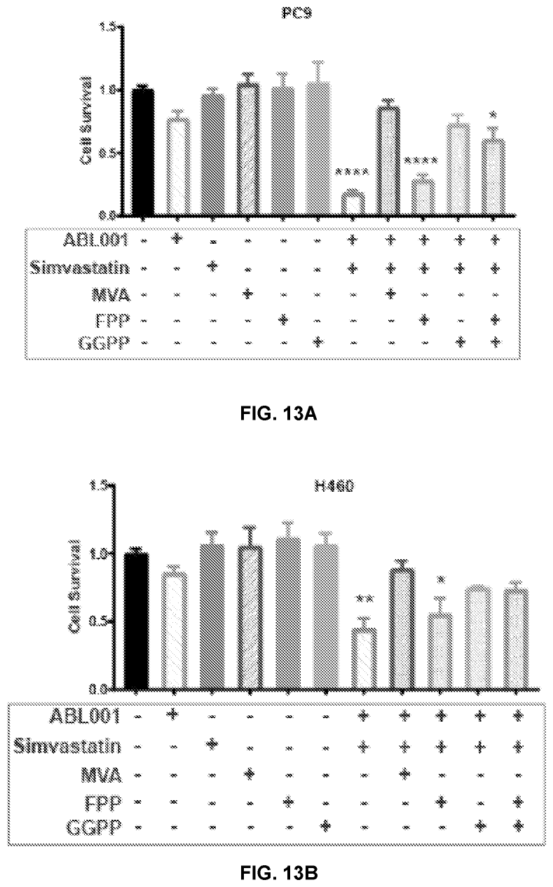

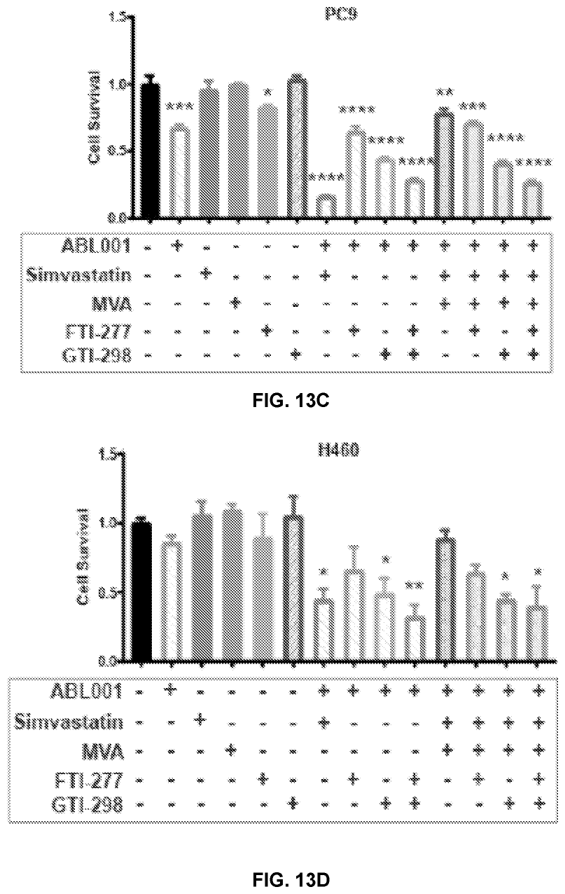

Mevalonate is the precursor to farnesyl diphosphate (FPP), which can either be elongated to geranylgeranyl diphosphate (GGPP) or cyclized to produce squalene for cholesterol production. Both FPP and GGPP are metabolites in the isoprenoid pathway required for protein prenylation, a posttranslational enzymatic modification that adds a prenylated motif to CAAX proteins, such as the RAP1A GTPase. These modifications regulate protein localization to different cellular compartments, facilitate specific protein-protein interactions and modulate protein stability. Since the downstream sterol metabolite cholesterol did not rescue cell survival, it was investigated whether metabolites in the isoprenoid pathway were critical for sensitization to statin therapeutics. It was found that addition of GGPP preferentially rescued cell viability compared to FPP in PC9 GR4, PC9, and H460 cells co-treated with ABL001 and simvastatin (FIG. 6A; FIGS. 13A and 13B). Immunoblotting was performed to assess whether protein prenylation was altered following simvastatin treatment (FIG. 6B). Simvastatin treatment increased levels of unprenylated RAP1A protein indicating inhibition of the geranylgeranylation pathway and induced a mobility shift in HDJ2 signifying inhibition of the farnesylation pathway, both of which were reversed by the addition of the indicated prenylation metabolites (FIG. 6B).

It was next tested whether inhibition of either geranylgeranyl transferase (GGT) or farnesyl transferase (FT) could impact cell survival in a manner similar to simvastatin treatment. Survival of PC9 GR4, PC9, and H460 cells, co-treated with ABL001 and the GGT-1 inhibitor (GGTI-298) was significantly impaired, but cell survival was only slightly decreased following addition of the FT inhibitor (FTI-277) in the presence of ABL001. Further, the non-additivity observed for statin treatment and GGT and FT inhibition, suggested that the synergizing effects of statins or GGTI-298+FTI-277 in the presence of ABL allosteric inhibitor operate through the same pathway (FIG. 6C; FIGS. 13C-13D). Immunoblotting confirmed that each inhibitor specifically suppressed its target pathway (FIG. 6D). Collectively, these data reveal that inhibition of protein geranylgeranylation is sufficient to sensitize cells to ABL allosteric inhibitors leading to enhanced intrinsic apoptosis.

As it was observed that oxidative metabolism was impaired following treatment with IC50 doses of the ABL allosteric inhibitors (Example 2) and that combination therapy induced MOMP (Example 4), it was investigated whether combination treatment with an ABL allosteric inhibitor and a statin affected mitochondrial metabolism and whether these effects might be due to changes in the protein prenylation pathway. To this end, changes in mitochondrial respiration was examined in cells cotreated with low doses of ABL001 and simvastatin and found that basal and maximal respiration as well as ATP production were decreased (FIG. 6E). Notably, the addition of MVA restored mitochondrial respiration back to baseline; while treatment with FTI-277 had minimal effect, treatment with GGTI-298 negated the MVA rescue and caused mitochondrial respiration levels to decrease to a similar degree as to those observed following ABL001 and simvastatin treatment (FIG. 6E). These data suggest that inhibition of the MVA pathway in combination with ABL kinase inhibition promotes cell death by impairing mitochondrial function.

### Example 6. Both Lung and Breast Cancer Cells Undergo Apoptotic Sensitization to Statin Therapy by ABL Allosteric Inhibitors

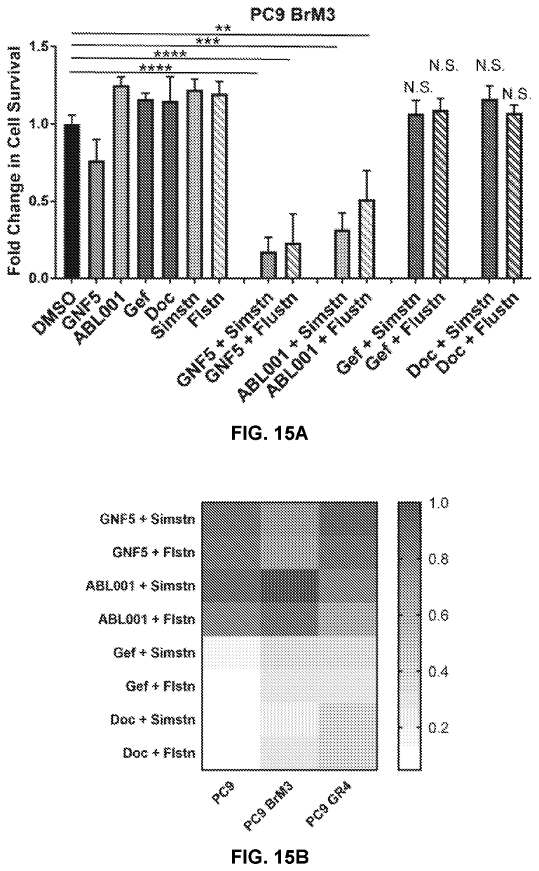

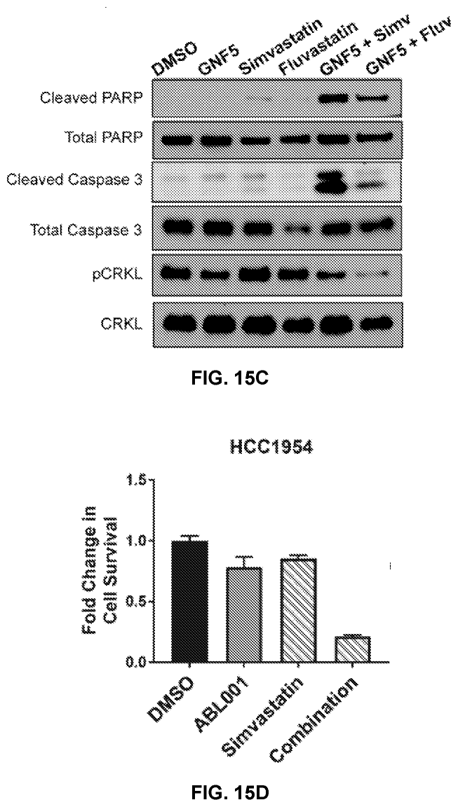

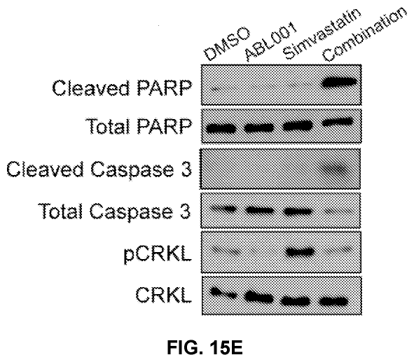

Treatment of PC9-BrM3 lung cancer cells with sub-therapeutic doses of GNF5 or ABL001 (1/2 of the calculated IC50 values) decreased cell survival in the presence of statins at sub-therapeutic doses that were 1/4 of their calculated IC50 values (FIG. 15A). Synergy scores showed that brain metastatic lung cancer cells were more sensitive to the combination of ABL001 with statins vs. parental cells (FIG. 15B). Interestingly, treatment of brain metastatic lung cancer cells with statins in combination with an EGFR TKI (Gefitinib) or chemotherapy (docetaxel) did not affect cell survival (FIGS. 15A-15B). Thus, ABL allosteric inhibitors preferentially sensitize brain metastatic cells to cell death in the presence of lipophilic statins. We found upregulation of cleaved PARP and cleaved caspase-3 apoptotic markers in cells treated with combination of the ABL inhibitor with either Simvastatin or Fluvastatin (FIG. 15C). Mitochondrial integrity was impaired following co-treatment with ABL001+statins as shown by FACS analysis of TMRM, a marker of mitochondrial membrane potential (data not shown). Notably, co-treatment of HCC1954 breast cancer cells with sub-therapeutic doses of ABL001 and Simvastatin resulted in a profound decrease in viability (FIG. 15D) and increased expression of apoptotic markers (FIG. 15E). Consistent with our data, epidemiological data suggest that use of lipophilic statins is associated with a reduced risk of breast cancer recurrence among postmenopausal women diagnosed with early stage breast cancer who received aromatase inhibitors in the adjuvant setting.

### Example 7. Combination Therapy of ABL001 and Simvastatin Impairs Tumor Growth and Increases Survival in Mouse Models of Lung Cancer Brain Metastasis and Gefitinib Resistance

Despite recent clinical successes with next-generation EGFR TKIs such as osimertinib, relapses occur for patients harboring EGFR mutant NSCLC. Moreover, patients harboring KRAS driver mutations have few tractable therapeutic options available. Further, the ability of anti-cancer drugs to efficiently penetrate the blood-brain barrier (BBB) and reach therapeutic doses for lung cancer patients harboring brain metastases is limited. Thus, it was chosen to evaluate whether statins could synergize with ABL inhibitors in vivo to treat cancer cells seeded at distal sites following intracardiac injection in clinically relevant mouse models of brain metastasis and therapy-resistance. ABL001 was employed as it has been shown to cross the BBB in preclinical mouse models and is currently in clinical trials for therapy-resistant patients with BCR-ABL+chronic myeloid leukemia. Importantly, administration of ABL001 by oral gavage is well tolerated and does not induce weight loss in mice. Pharmacokinetic data has shown that lipophilic statins can cross the BBB more readily than hydrophilic statins. In this regard, studies testing the ability of radiolabeled simvastatin to cross the BBB identified simvastatin-derived radioactivity in the rat brain following oral administration. Thus, we employed clinically relevant low doses of simvastatin, and treated mice with 10 mg/kd QD simvastatin, which is equivalent to doses used in humans.

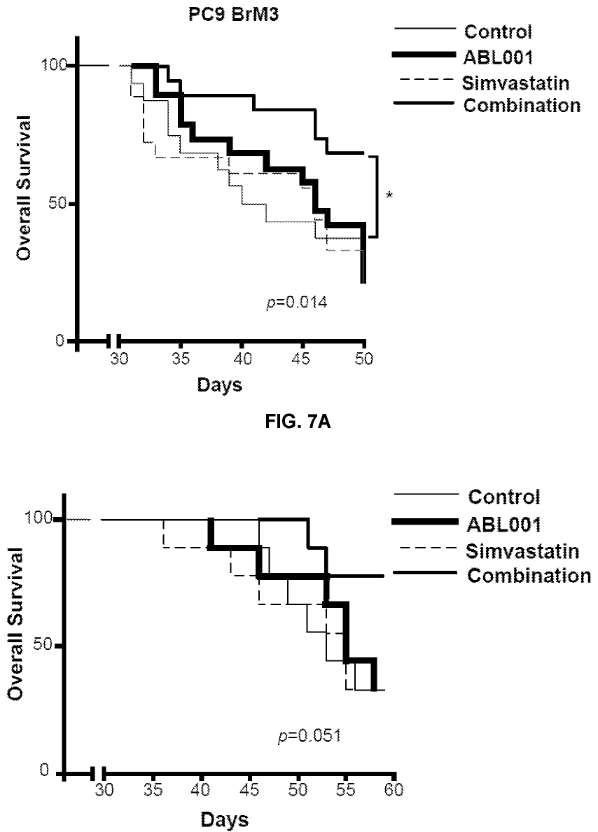

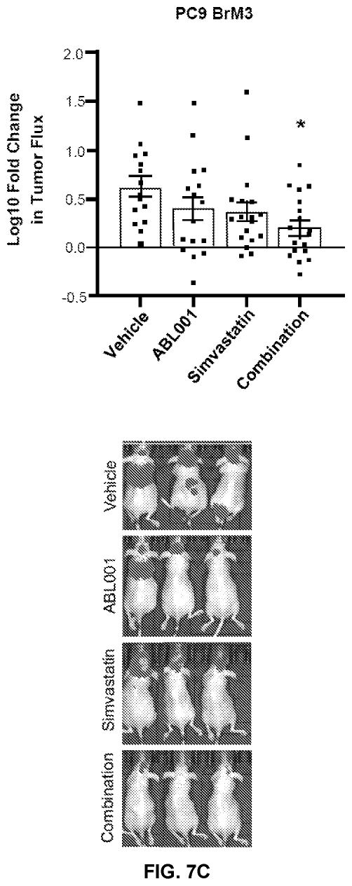

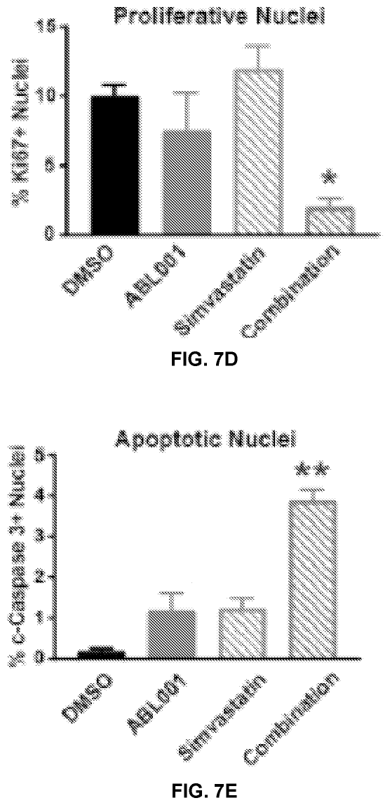

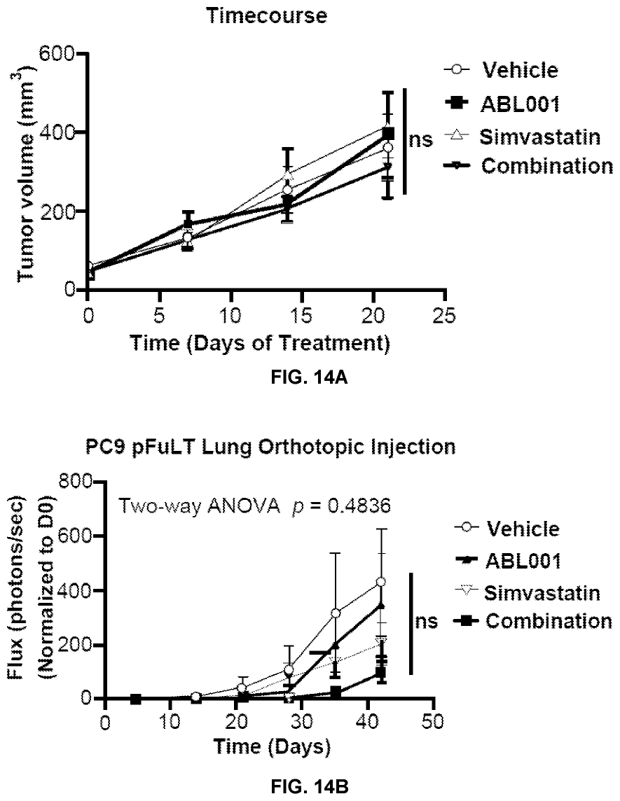

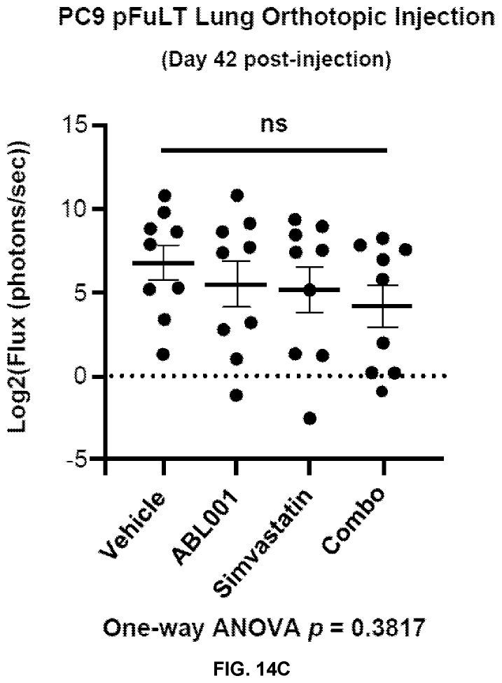

To determine whether combination treatment could impair brain metastatic outgrowth, brain-metastatic PC9-BrM3 cells derived through serial rounds of intracardiac injection in athymic nude mice were used. Previous studies have shown that following injection into the arterial circulation, brain-metastatic lung cancer cells extravasate into the brain parenchyma by day 6 post-injection. Thus, to evaluate the effectiveness of combination ABL001 and statin treatment on metastatic colonization, bioluminescent imaging (BLI) was performed on day 6 post-intracardiac injection to stratify mice into treatment groups and began drug treatments on day 7. Mice were divided into four treatment groups: vehicle, ABL001, simvastatin, or combination of ABL001 and simvastatin. It was found that overall survival was significantly increased in mice harboring PC9-BrM3 brain metastases following combination treatment in comparison to vehicle, ABL001, or simvastatin alone (FIG. 7A). Similar results were also observed in mice harboring gefitinib resistant PC9 cells (PC9 GR4), with ABL001 and simvastatin combination therapy extending their overall survival (FIG. 7B). Quantification of brain flux at day 27 revealed decreased disease burden in mice treated with both ABL001 and simvastatin compared to vehicle or single drug treatment groups (FIG. 7C). Immunofluorescence analysis of brain metastases for proliferation and apoptotic markers revealed decreased expression of the proliferative marker Ki67 and increased expression of the cell death marker cleaved caspase-3 (FIGS. 7D-7E; data not shown). Notably, the effect of combination drug treatment on subcutaneous xenograft tumor growth was minimal in comparison to the vehicle or single treatment groups for PC9 GR4 cells injected subcutaneously into the flank (FIG. 14A). Quantification of vehicle- and drug-treated mice harboring orthotopic lung tumors following intrathoracic injections showed that the combination of ABL001 and simvastatin elicited a decrease in tumor burden, but this did not reach statistical significance (FIGS. 14B-14C). These findings are consistent with previous reports showing that the inhibitory effects of ABL inactivation on primary tumor growth are cell context dependent and that genetic and pharmacologic inhibition of the ABL kinases predominantly decreased spontaneous metastasis. Together, these data reveal that combination treatment with both ABL001 and simvastatin impairs metastatic outgrowth of lung cancer cells by enhancing tumor cell death.

### Example 8. ABL Kinase Inhibition Impairs Outgrowth of HER2+ Breast Cancer Brain Metastatic Cells and Improve Overall Survival of Tumor-Bearing Mice

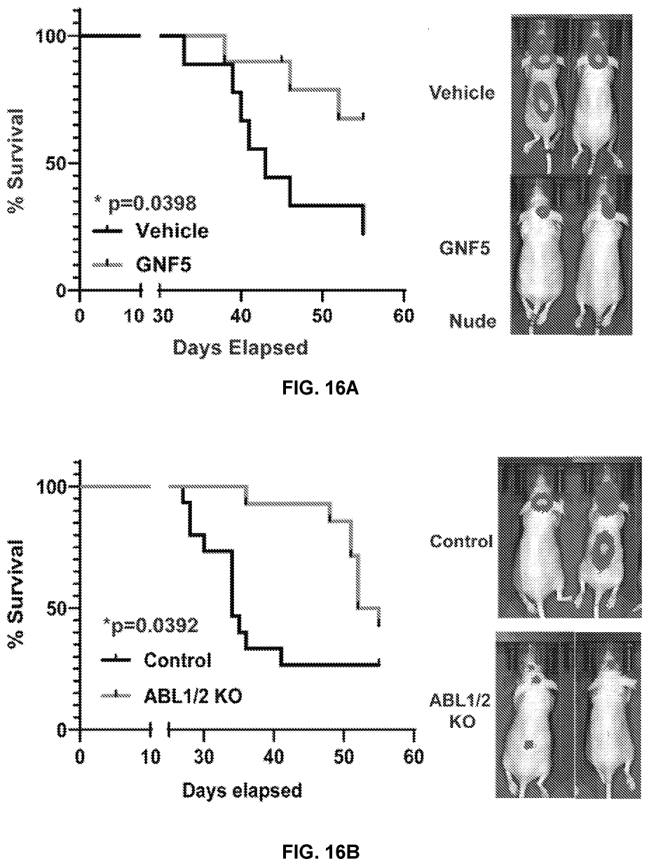

ABL allosteric inhibitors have been shown to cross the blood brain barrier (BBB) and are effective in treating lung cancer brain metastases in mouse models (Hoj, Mayro and Pendergast 2019 Cell Reports). Thus, it was evaluated whether ABL allosteric inhibitors might be effective in treating HER2+ breast cancer colonization of the brain, which is the limiting step in the metastatic cascade. Following intracranial injection of brain metastatic HCC1954-LCC1 breast cancer cells, mice harboring brain metastases were treated by oral gavage with the ABL allosteric inhibitor GNF5, which resulted in impaired metastatic outgrowth and colonization of the brain parenchyma as measured by bioluminescence imaging (BLI), and markedly increased animal survival (FIG. 16). To ascertain that drug effects were on target, ABL kinases were depleted in HCC1954-LCC1 cells using CRISPR-Cas9. Knockout of ABL1+ABL2 markedly reduced brain colonization and improved overall survival (FIG. 16B). These data support a role for ABL kinases in promoting colonization of the brain by HER2+ breast cancer cells, and show that ABL allosteric inhibitors effectively penetrate the BBB and inhibit intracranial growth.

TABLE S2
Gene-Level Three Scores from CRISPR/Cas9 Screen

| Genes | Descriptive Gene Name | log2 TS |
|---|---|---|
| A4GALT | alpha 1,4-galactosyltransferase(A4GALT) | −0.0548 |
| AACS | acetoacetyl-CoA synthetase(AACS) | −0.07047 |
| AADAC | arylacetamide deacetylase(AADAC) | −0.12851 |
| AADAT | aminoadipate aminotransferase(AADAT) | −0.04222 |
| AASS | aminoadipate-semialdehyde synthase(AASS) | −0.14817 |
| ABAT | 4-aminobutyrate aminotransferase(ABAT) | 0.087318 |
| ABCA1 | ATP binding cassette subfamily A member 1(ABCA1) | 0.05348 |
| ABCA10 | ATP binding cassette subfamily A member 10(ABCA10) | −0.09416 |
| ABCA12 | ATP binding cassette subfamily A member 12(ABCA12) | −0.08821 |
| ABCA13 | ATP binding cassette subfamily A member 13(ABCA13) | 0.010711 |
| ABCA2 | ATP binding cassette subfamily A member 2(ABCA2) | −0.21022 |
| ABCA3 | ATP binding cassette subfamily A member 3(ABCA3) | −0.24682 |
| ABCA4 | ATP binding cassette subfamily A member 4(ABCA4) | −0.40937 |
| ABCA5 | ATP binding cassette subfamily A member 5(ABCA5) | −0.15441 |
| ABCA6 | ATP binding cassette subfamily A member 6(ABCA6) | 0.006517 |
| ABCA7 | ATP binding cassette subfamily A member 7(ABCA7) | −0.00498 |
| ABCA8 | ATP binding cassette subfamily A member 8(ABCA8) | −0.09945 |
| ABCA9 | ATP binding cassette subfamily A member 9(ABCA9) | −0.31431 |
| ABCB1 | ATP binding cassette subfamily B member 1(ABCB1) | −0.26501 |
| ABCB10 | ATP binding cassette subfamily B member 10(ABCB10) | −0.44957 |
| ABCB11 | ATP binding cassette subfamily B member 11(ABCB11) | −0.46443 |
| ABCB4 | ATP binding cassette subfamily B member 4(ABCB4) | 0.140538 |
| ABCB5 | ATP binding cassette subfamily B member 5(ABCB5) | −0.08867 |
| ABCB6 | ATP binding cassette subfamily B member 6 (Langereis blood group)(ABCB6) | −0.51176 |
| ABCB7 | ATP binding cassette subfamily B member 7(ABCB7) | −0.17983 |
| ABCB8 | ATP binding cassette subfamily B member 8(ABCB8) | −0.02223 |
| ABCB9 | ATP binding cassette subfamily B member 9(ABCB9) | 0.050842 |
| ABCC1 | ATP binding cassette subfamily C member 1(ABCC1) | −0.49868 |
| ABCC10 | ATP binding cassette subfamily C member 10(ABCC10) | −0.22696 |
| ABCC11 | ATP binding cassette subfamily C member 11(ABCC11) | −0.46683 |
| ABCC12 | ATP binding cassette subfamily C member 12(ABCC12) | −0.03014 |
| ABCC2 | ATP binding cassette subfamily C member 2(ABCC2) | −0.33152 |
| ABCC3 | ATP binding cassette subfamily C member 3(ABCC3) | −0.04848 |
| ABCC4 | ATP binding cassette subfamily C member 4(ABCC4) | −0.15645 |
| ABCC5 | ATP binding cassette subfamily C member 5(ABCC5) | −0.27801 |
| ABCC6 | ATP binding cassette subfamily C member 6(ABCC6) | −0.12696 |
| ABCC8 | ATP binding cassette subfamily C member 8(ABCC8) | −0.14074 |
| ABCC9 | ATP binding cassette subfamily C member 9(ABCC9) | 0.161825 |
| ABCD1 | ATP binding cassette subfamily D member 1(ABCD1) | −0.25673 |
| ABCD2 | ATP binding cassette subfamily D member 2(ABCD2) | −0.10786 |
| ABCD3 | ATP binding cassette subfamily D member 3(ABCD3) | 0.056186 |
| ABCD4 | ATP binding cassette subfamily D member 4(ABCD4) | 0.078528 |
| ABCE1 | ATP binding cassette subfamily E member 1(ABCE1) | −0.4113 |
| ABCF1 | ATP binding cassette subfamily F member 1(ABCF1) | 0.143533 |
| ABCF2 | ATP binding cassette subfamily F member 2(ABCF2) | −0.21415 |
| ABCF3 | ATP binding cassette subfamily F member 3(ABCF3) | −0.0403 |
| ABCG1 | ATP binding cassette subfamily G member 1(ABCG1) | −0.27853 |
| ABCG2 | ATP binding cassette subfamily G member 2 (Junior blood group)(ABCG2) | −0.27378 |
| ABCG4 | ATP binding cassette subfamily G member 4(ABCG4) | −0.04084 |
| ABCG5 | ATP binding cassette subfamily G member 5(ABCG5) | −0.22479 |
| ABCG8 | ATP binding cassette subfamily G member 8(ABCG8) | −0.13034 |
| ABHD10 | abhydrolase domain containing 10(ABHD10) | 0.018339 |
| ABHD2 | abhydrolase domain containing 2(ABHD2) | −0.39785 |
| ABHD3 | abhydrolase domain containing 3(ABHD3) | 0.082143 |
| ABHD4 | abhydrolase domain containing 4(ABHD4) | −0.15709 |
| ABHD5 | abhydrolase domain containing 5(ABHD5) | −0.28065 |
| ABHD6 | abhydrolase domain containing 6(ABHD6) | −0.11598 |
| ABHD8 | abhydrolase domain containing 8(ABHD8) | −0.35452 |
| ACAA1 | acetyl-CoA acyltransferase 1(ACAA1) | −0.19841 |
| ACAA2 | acetyl-CoA acyltransferase 2(ACAA2) | −0.00667 |
| ACACA | acetyl-CoA carboxylase alpha(ACACA) | 0.115115 |
| ACACB | acetyl-CoA carboxylase beta(ACACB) | 0.05836 |
| ACAD10 | acyl-CoA dehydrogenase family member 10(ACAD10) | −0.12025 |
| ACAD11 | acyl-CoA dehydrogenase family member 11(ACAD11) | −0.44036 |
| ACAD8 | acyl-CoA dehydrogenase family member 8(ACAD8) | −0.13775 |
| ACAD9 | acyl-CoA dehydrogenase family member 9(ACAD9) | −0.08889 |
| ACADL | acyl-CoA dehydrogenase, long chain(ACADL) | −0.29298 |
| ACADM | acyl-CoA dehydrogenase, C-4 to C-12 straight chain(ACADM) | −0.31199 |
| ACADS | acyl-CoA dehydrogenase, C-2 to C-3 short chain(ACADS) | −0.22986 |
| ACADSB | acyl-CoA dehydrogenase, short/branched chain(ACADSB) | 0.04496 |
| ACADVL | acyl-CoA dehydrogenase, very long chain(ACADVL) | −0.3465 |
| ACAT1 | acetyl-CoA acetyltransferase 1(ACAT1) | −0.09943 |
| ACAT2 | acetyl-CoA acetyltransferase 2(ACAT2) | −0.04443 |
| ACBD3 | acyl-CoA binding domain containing 3(ACBD3) | −0.45615 |
| ACBD4 | acyl-CoA binding domain containing 4(ACBD4) | −0.18115 |
| ACBD5 | acyl-CoA binding domain containing 5(ACBD5) | −0.6591 |
| ACBD6 | acyl-CoA binding domain containing 6(ACBD6) | −0.22042 |
| ACBD7 | acyl-CoA binding domain containing 7(ACBD7) | −0.18335 |
| ACE | angiotensin I converting enzyme(ACE) | −0.00201 |
| ACE2 | angiotensin I converting enzyme 2(ACE2) | −0.58658 |
| ACER1 | alkaline ceramidase 1(ACER1) | 0.127137 |
| ACER2 | alkaline ceramidase 2(ACER2) | −0.16153 |
| ACER3 | alkaline ceramidase 3(ACER3) | 0.061753 |
| ACHE | acetylcholinesterase (Cartwright blood group)(ACHE) | −0.41637 |
| ACLY | ATP citrate lyase(ACLY) | −0.46955 |
| ACO1 | aconitase 1(ACO1) | −0.07986 |
| ACO2 | aconitase 2(ACO2) | −0.28725 |
| ACOT1 | acyl-CoA thioesterase 1(ACOT1) | −0.03665 |
| ACOT11 | acyl-CoA thioesterase 11(ACOT11) | −0.18417 |
| ACOT12 | acyl-CoA thioesterase 12(ACOT12) | −0.42041 |
| ACOT2 | acyl-CoA thioesterase 2(ACOT2) | −0.10849 |
| ACOT4 | acyl-CoA thioesterase 4(ACOT4) | −0.04057 |
| ACOT6 | acyl-CoA thioesterase 6(ACOT6) | −0.22903 |
| ACOT7 | acyl-CoA thioesterase 7(ACOT7) | 0.100039 |
| ACOT8 | acyl-CoA thioesterase 8(ACOT8) | −0.03184 |
| ACOT9 | acyl-CoA thioesterase 9(ACOT9) | −0.13798 |
| ACOX1 | acyl-CoA oxidase 1(ACOX1) | −0.45482 |
| ACOX2 | acyl-CoA oxidase 2(ACOX2) | −0.28001 |
| ACOX3 | acyl-CoA oxidase 3, pristanoyl(ACOX3) | −0.38397 |
| ACOXL | acyl-CoA oxidase-like(ACOXL) | 0.164157 |
| ACP1 | acid phosphatase 1, soluble(ACP1) | −0.1503 |
| ACP2 | acid phosphatase 2, lysosomal(ACP2) | −0.14242 |
| ACSBG1 | acyl-CoA synthetase bubblegum family member 1(ACSBG1) | −0.08408 |
| ACSBG2 | acyl-CoA synthetase bubblegum family member 2(ACSBG2) | −0.45815 |
| ACSF2 | acyl-CoA synthetase family member 2(ACSF2) | −0.27453 |
| ACSF3 | acyl-CoA synthetase family member 3(ACSF3) | −0.1766 |
| ACSL1 | acyl-CoA synthetase long-chain family member 1(ACSL1) | −0.02883 |
| ACSL3 | acyl-CoA synthetase long-chain family member 3(ACSL3) | −0.3983 |
| ACSL4 | acyl-CoA synthetase long-chain family member 4(ACSL4) | −0.19617 |
| ACSL5 | acyl-CoA synthetase long-chain family member 5(ACSL5) | −0.02567 |
| ACSL6 | acyl-CoA synthetase long-chain family member 6(ACSL6) | −0.08578 |
| ACSM1 | acyl-CoA synthetase medium-chain family member 1(ACSM1) | −0.15474 |
| ACSM2A | acyl-CoA synthetase medium-chain family member 2A(ACSM2A) | −0.15893 |
| ACSM2B | acyl-CoA synthetase medium-chain family member 2B(ACSM2B) | 0.105694 |
| ACSM3 | acyl-CoA synthetase medium-chain family member 3(ACSM3) | −0.2869 |
| ACSM4 | acyl-CoA synthetase medium-chain family member 4(ACSM4) | −0.04692 |
| ACSM5 | acyl-CoA synthetase medium-chain family member 5(ACSM5) | −0.28894 |
| ACSS1 | acyl-CoA synthetase short-chain family member 1(ACSS1) | −0.27746 |
| ACSS2 | acyl-CoA synthetase short-chain family member 2(ACSS2) | 0.216067 |
| ACSS3 | acyl-CoA synthetase short-chain family member 3(ACSS3) | −0.09541 |
| ACY1 | aminoacylase 1(ACY1) | −0.25534 |
| ACY3 | aminoacylase 3(ACY3) | −0.13247 |
| ACYP1 | acylphosphatase 1(ACYP1) | 0.119099 |
| ACYP2 | acylphosphatase 2(ACYP2) | 0.030119 |
| ADA | adenosine deaminase(ADA) | −0.05297 |
| ADAD1 | adenosine deaminase domain containing 1(ADAD1) | −0.54176 |
| ADAD2 | adenosine deaminase domain containing 2(ADAD2) | −0.1572 |
| ADAL | adenosine deaminase like(ADAL) | −0.22736 |
| ADC | | 0.026006 |
| ADCK3 | | −0.0344 |
| ADCY1 | adenylate cyclase 1(ADCY1) | 0.31277 |
| ADCY10 | adenylate cyclase 10, soluble(ADCY10) | −0.13109 |
| ADCY2 | adenylate cyclase 2(ADCY2) | −0.30417 |
| ADCY3 | adenylate cyclase 3(ADCY3) | −0.03144 |
| ADCY4 | adenylate cyclase 4(ADCY4) | 0.002648 |
| ADCY5 | adenylate cyclase 5(ADCY5) | −0.0498 |
| ADCY6 | adenylate cyclase 6(ADCY6) | −0.04535 |
| ADCY7 | adenylate cyclase 7(ADCY7) | −0.27772 |
| ADCY8 | adenylate cyclase 8(ADCY8) | −0.04847 |
| ADCY9 | adenylate cyclase 9(ADCY9) | −0.10534 |
| ADH1A | alcohol dehydrogenase 1A (class I), alpha polypeptide(ADH1A) | −0.20152 |
| ADH1B | alcohol dehydrogenase 1B (class I), beta polypeptide(ADH1B) | 0.077414 |
| ADH4 | alcohol dehydrogenase 4 (class II), pi polypeptide(ADH4) | −0.4213 |
| ADH5 | alcohol dehydrogenase 5 (class III), chi polypeptide(ADH5) | −0.24621 |
| ADH6 | alcohol dehydrogenase 6 (class V)(ADH6) | −0.2976 |
| ADH7 | alcohol dehydrogenase 7 (class IV), mu or sigma polypeptide(ADH7) | 0.109518 |
| ADI1 | acireductone dioxygenase 1(ADI1) | −0.13503 |
| ADK | adenosine kinase(ADK) | −0.17282 |
| ADPGK | ADP dependent glucokinase(ADPGK) | −0.20816 |
| ADSL | adenylosuccinate lyase(ADSL) | −1.30126 |
| ADSS | adenylosuccinate synthase(ADSS) | 0.70165 |
| ADSSL1 | adenylosuccinate synthase like 1(ADSSL1) | −0.0122 |
| AGA | aspartylglucosaminidase(AGA) | −0.28206 |
| AGK | acylglycerol kinase(AGK) | 0.086732 |
| AGL | amylo-alpha-1, 6-glucosidase, 4-alpha-glucanotransferase(AGL) | −0.14266 |
| AGPAT1 | 1-acylglycerol-3-phosphate O-acyltransferase 1(AGPAT1) | −0.43255 |
| AGPAT2 | 1-acylglycerol-3-phosphate O-acyltransferase 2(AGPAT2) | −0.10348 |
| AGPS | alkylglycerone phosphate synthase(AGPS) | −0.21246 |
| AGXT | alanine-glyoxylate aminotransferase(AGXT) | −0.32566 |
| AGXT2 | alanine--glyoxylate aminotransferase 2(AGXT2) | −0.1448 |
| AHCY | adenosylhomocysteinase(AHCY) | −0.59912 |
| AHCYL1 | adenosylhomocysteinase like 1(AHCYL1) | −0.20986 |
| AHCYL2 | adenosylhomocysteinase like 2(AHCYL2) | −0.20781 |
| AHR | aryl hydrocarbon receptor(AHR) | 0.01358 |
| AIPL1 | aryl hydrocarbon receptor interacting protein like 1(AIPL1) | −0.09546 |
| AK1 | adenylate kinase 1(AK1) | −0.17236 |
| AK2 | adenylate kinase 2(AK2) | −0.20706 |
| AK3 | adenylate kinase 3(AK3) | −0.17478 |
| AK5 | adenylate kinase 5(AK5) | 0.02394 |
| AK7 | adenylate kinase 7(AK7) | −0.07438 |
| AKR1A1 | aldo-keto reductase family 1 member A1(AKR1A1) | −0.66089 |
| AKR1B1 | aldo-keto reductase family 1 member B(AKR1B1) | −0.06727 |
| AKR1B10 | aldo-keto reductase family 1 member B10(AKR1B10) | 0.079509 |
| AKR1C1 | aldo-keto reductase family 1 member C1(AKR1C1) | −0.00726 |
| AKR1C2 | aldo-keto reductase family 1 member C2(AKR1C2) | −0.15228 |
| AKR1C3 | aldo-keto reductase family 1 member C3(AKR1C3) | −0.33759 |
| AKR1C4 | aldo-keto reductase family 1 member C4(AKR1C4) | −0.1468 |
| AKR1D1 | aldo-keto reductase family 1 member D1(AKR1D1) | −0.10706 |
| AKR7A2 | aldo-keto reductase family 7 member A2(AKR7A2) | −0.14265 |
| AKR7A3 | aldo-keto reductase family 7 member A3(AKR7A3) | 0.27812 |
| ALAD | aminolevulinate dehydratase(ALAD) | −0.34967 |
| ALAS1 | 5′-aminolevulinate synthase 1(ALAS1) | 0.11615 |
| ALAS2 | 5′-aminolevulinate synthase 2(ALAS2) | 0.055527 |
| ALDH16A1 | aldehyde dehydrogenase 16 family member A1(ALDH16A1) | −0.46845 |
| ALDH18A1 | aldehyde dehydrogenase 18 family member A1(ALDH18A1) | −0.00227 |
| ALDH1A1 | aldehyde dehydrogenase 1 family member A1(ALDH1A1) | −0.18157 |
| ALDH1A2 | aldehyde dehydrogenase 1 family member A2(ALDH1A2) | −0.07068 |
| ALDH1A3 | aldehyde dehydrogenase 1 family member A3(ALDH1A3) | −0.10414 |
| ALDH1B1 | aldehyde dehydrogenase 1 family member B1(ALDH1B1) | −0.09746 |
| ALDH1L1 | aldehyde dehydrogenase 1 family member L1(ALDH1L1) | −0.08117 |
| ALDH1L2 | aldehyde dehydrogenase 1 family member L2(ALDH1L2) | −0.61917 |
| ALDH2 | aldehyde dehydrogenase 2 family (mitochondrial)(ALDH2) | −0.18317 |
| ALDH3A1 | aldehyde dehydrogenase 3 family member A1(ALDH3A1) | −0.34977 |
| ALDH3A2 | aldehyde dehydrogenase 3 family member A2(ALDH3A2) | −0.01328 |
| ALDH3B2 | aldehyde dehydrogenase 3 family member B2(ALDH3B2) | −0.43437 |
| ALDH4A1 | aldehyde dehydrogenase 4 family member A1(ALDH4A1) | −0.05261 |
| ALDH5A1 | aldehyde dehydrogenase 5 family member A1(ALDH5A1) | −0.18917 |
| ALDH6A1 | aldehyde dehydrogenase 6 family member A1(ALDH6A1) | 0.162471 |
| ALDH7A1 | aldehyde dehydrogenase 7 family member A1(ALDH7A1) | −0.17121 |
| ALDH8A1 | aldehyde dehydrogenase 8 family member A1(ALDH8A1) | −0.28275 |
| ALDH9A1 | aldehyde dehydrogenase 9 family member A1(ALDH9A1) | −0.35156 |
| ALDOA | aldolase, fructose-bisphosphate A(ALDOA) | 0.094024 |
| ALDOB | aldolase, fructose-bisphosphate B(ALDOB) | −0.2395 |
| ALDOC | aldolase, fructose-bisphosphate C(ALDOC) | 0.006619 |
| ALG3 | ALG3, alpha-1,3-mannosyltransferase(ALG3) | −0.28711 |
| ALG6 | ALG6, alpha-1,3-glucosyltransferase(ALG6) | −0.17771 |
| ALOX12 | arachidonate 12-lipoxygenase, 12S type(ALOX12) | −0.32091 |
| ALOX12B | arachidonate 12-lipoxygenase, 12R type(ALOX12B) | 0.106955 |
| ALOX15 | arachidonate 15-lipoxygenase(ALOX15) | −0.4068 |
| ALOX15B | arachidonate 15-lipoxygenase, type B(ALOX15B) | −0.25888 |
| ALOX5 | arachidonate 5-lipoxygenase(ALOX5) | −0.31305 |
| ALOX5AP | arachidonate 5-lipoxygenase activating protein(ALOX5AP) | −0.2302 |
| ALOXE3 | arachidonate lipoxygenase 3(ALOXE3) | −0.35074 |
| ALX3 | ALX homeobox 3(ALX3) | −0.38103 |
| AMACR | alpha-methylacyl-CoA racemase(AMACR) | −0.16293 |
| AMD1 | adenosylmethionine decarboxylase 1(AMD1) | −0.17028 |
| AMDHD1 | amidohydrolase domain containing 1(AMDHD1) | −0.02372 |
| AMPD1 | adenosine monophosphate deaminase 1(AMPD1) | −0.04263 |
| AMPD2 | adenosine monophosphate deaminase 2(AMPD2) | −0.33321 |
| AMPD3 | adenosine monophosphate deaminase 3(AMPD3) | −0.13795 |
| AMT | aminomethyltransferase(AMT) | −0.10256 |
| AMY1A | amylase, alpha 1A (salivary)(AMY1A) | −0.03793 |
| AMY1B | amylase, alpha 1B (salivary)(AMY1B) | −0.03793 |
| AMY1C | amylase, alpha 1C (salivary)(AMY1C) | −0.03793 |
| AMY2A | amylase, alpha 2A (pancreatic)(AMY2A) | −0.01633 |
| AMY2B | amylase, alpha 2B (pancreatic)(AMY2B) | −0.21439 |
| ANKH | ANKH inorganic pyrophosphate transport regulator(ANKH) | −0.13945 |
| AOAH | acyloxyacyl hydrolase(AOAH) | −0.08497 |
| AOC1 | amine oxidase, copper containing 1(AOC1) | −0.07104 |
| AOX1 | aldehyde oxidase 1(AOX1) | −0.2825 |
| APEH | acylaminoacyl-peptide hydrolase(APEH) | −0.06698 |
| APRT | adenine phosphoribosyltransferase(APRT) | 0.017907 |
| AQP1 | aquaporin 1 (Colton blood group)(AQP1) | −0.04668 |
| AQP2 | aquaporin 2(AQP2) | −0.09327 |
| AQP3 | aquaporin 3 (Gill blood group)(AQP3) | −0.08783 |
| AQP4 | aquaporin 4(AQP4) | −0.30836 |
| AQP5 | aquaporin 5(AQP5) | −0.03167 |
| AQP6 | aquaporin 6(AQP6) | −0.12047 |
| AQP7 | aquaporin 7(AQP7) | −0.20549 |
| AQP8 | aquaporin 8(AQP8) | −0.02709 |
| AQP9 | aquaporin 9(AQP9) | −0.24632 |
| AR | androgen receptor(AR) | −0.13979 |
| ARG1 | arginase 1(ARG1) | −0.65155 |
| ARSA | arylsulfatase A(ARSA) | −0.15768 |
| ARSB | arylsulfatase B(ARSB) | −0.25239 |
| ARSD | arylsulfatase D(ARSD) | −0.43419 |
| ARSE | arylsulfatase E (chondrodysplasia punctata 1)(ARSE) | −0.33286 |
| ARSF | arylsulfatase F(ARSF) | −0.21099 |
| ARSG | arylsulfatase G(ARSG) | −0.46637 |
| ARSH | arylsulfatase family member H(ARSH) | 0.17036 |
| ARSI | arylsulfatase family member I(ARSI) | −0.39565 |
| ARSJ | arylsulfatase family member J(ARSJ) | −0.19193 |
| ARSK | arylsulfatase family member K(ARSK) | −0.1794 |
| AS3MT | arsenite methyltransferase(AS3MT) | −0.14014 |
| ASAH1 | N-acylsphingosine amidohydrolase 1(ASAH1) | −0.37661 |
| ASAH2 | N-acylsphingosine amidohydrolase 2(ASAH2) | −0.28187 |
| ASAH2B | N-acylsphingosine amidohydrolase 2B(ASAH2B) | −0.55626 |
| ASIC1 | acid sensing ion channel subunit 1(ASIC1) | −0.15293 |
| ASIC2 | acid sensing ion channel subunit 2(ASIC2) | −0.06803 |
| ASIC3 | acid sensing ion channel subunit 3(ASIC3) | 0.01586 |
| ASIC4 | acid sensing ion channel subunit family member 4(ASIC4) | −0.27652 |
| ASIC5 | acid sensing ion channel subunit family member 5(ASIC5) | 0.007914 |
| ASL | argininosuccinate lyase(ASL) | 0.027926 |
| ASMT | acetylserotonin O-methyltransferase(ASMT) | −0.34095 |
| ASMTL | acetylserotonin O-methyltransferase-like(ASMTL) | −0.3519 |
| ASNS | asparagine synthetase (glutamine-hydrolyzing)(ASNS) | −0.2272 |
| ASNSD1 | asparagine synthetase domain containing 1(ASNSD1) | −0.42477 |
| ASPA | aspartoacylase(ASPA) | −0.00646 |
| ASPG | asparaginase(ASPG) | −0.14433 |
| ASRGL1 | asparaginase like 1(ASRGL1) | −0.17504 |
| ASS1 | argininosuccinate synthase 1(ASS1) | −0.23805 |
| ATIC | 5-aminoimidazole-4-carboxamide ribonucleotide formyltransferase/IMP cyclc [symbol] | −1.15819 |
| ATOH1 | atonal bHLH transcription factor 1(ATOH1) | −0.47875 |
| ATP11B | ATPase phospholipid transporting 11B (putative)(ATP11B) | −0.2463 |
| ATP12A | ATPase H+/K+ transporting non-gastric alpha2 subunit(ATP12A) | −0.17361 |
| ATP13A2 | ATPase 13A2(ATP13A2) | −0.1777 |
| ATP13A3 | ATPase 13A3(ATP13A3) | 0.084532 |
| ATP13A4 | ATPase 13A4(ATP13A4) | 0.43686 |
| ATP13A5 | ATPase 13A5(ATP13A5) | −0.20861 |
| ATP1A1 | ATPase Na+/K+ transporting subunit alpha 1(ATP1A1) | 0.016565 |
| ATP1A2 | ATPase Na+/K+ transporting subunit alpha 2(ATP1A2) | −0.29491 |
| ATP1A3 | ATPase Na+/K+ transporting subunit alpha 3(ATP1A3) | −0.22281 |
| ATP1A4 | ATPase Na+/K+ transporting subunit alpha 4(ATP1A4) | 0.11782 |
| ATP2A1 | ATPase sarcoplasmic/endoplasmic reticulum Ca2+ transporting 1(ATP2A1) | −0.10204 |
| ATP2A2 | ATPase sarcoplasmic/endoplasmic reticulum Ca2+ transporting 2(ATP2A2) | −0.20967 |
| ATP2A3 | ATPase sarcoplasmic/endoplasmic reticulum Ca2+ transporting 3(ATP2A3) | −0.27863 |
| ATP2B1 | ATPase plasma membrane Ca2+ transporting 1(ATP2B1) | −0.6689 |
| ATP2B2 | ATPase plasma membrane Ca2+ transporting 2(ATP2B2) | −0.37805 |
| ATP2B3 | ATPase plasma membrane Ca2+ transporting 3(ATP2B3) | −0.27284 |
| ATP2B4 | ATPase plasma membrane Ca2+ transporting 4(ATP2B4) | −0.20946 |
| ATP2C1 | ATPase secretory pathway Ca2+ transporting 1(ATP2C1) | −0.05352 |
| ATP2C2 | ATPase secretory pathway Ca2+ transporting 2(ATP2C2) | −0.0542 |
| ATP4A | ATPase H+/K+ transporting alpha subunit(ATP4A) | 0.007593 |
| ATP4B | ATPase H+/K+ transporting beta subunit(ATP4B) | −0.49557 |
| ATP5A1 | ATP synthase, H+ transporting, mitochondrial F1 complex, alpha subunit 1, ca [symbol] | −0.67188 |
| ATP5B | ATP synthase, H+ transporting, mitochondrial F1 complex, beta polypeptide(A [symbol] | −0.68843 |
| ATP5C1 | ATP synthase, H+ transporting, mitochondrial F1 complex, gamma polypeptid [symbol] | 0.215257 |
| ATP5D | ATP synthase, H+ transporting, mitochondrial F1 complex, delta subunit(ATP5 [symbol] | −0.57218 |
| ATP5E | ATP synthase, H+ transporting, mitochondrial F1 complex, epsilon subunit(AT [symbol] | 0.150107 |
| ATP5G3 | ATP synthase, H+ transporting, mitochondrial Fo complex subunit C3 (subunit [symbol] | 0.140576 |
| ATP5H | ATP synthase, H+ transporting, mitochondrial Fo complex subunit D(ATP5H) | −0.29148 |
| ATP5J | ATP synthase, H+ transporting, mitochondrial Fo complex subunit F6(ATP5J) | −0.25505 |
| ATP6AP2 | ATPase H+ transporting accessory protein 2(ATP6AP2) | −0.48193 |
| ATP6V0A4 | ATPase H+ transporting V0 subunit a4(ATP6V0A4) | −0.11048 |
| ATP6V1A | ATPase H+ transporting V1 subunit A(ATP6V1A) | −0.67425 |
| ATP6V1B1 | ATPase H+ transporting V1 subunit B1(ATP6V1B1) | −0.08149 |
| ATP6V1B2 | ATPase H+ transporting V1 subunit B2(ATP6V1B2) | −0.60104 |
| ATP6V1C1 | ATPase H+ transporting V1 subunit C1(ATP6V1C1) | −0.66354 |
| ATP6V1D | ATPase H+ transporting V1 subunit D(ATP6V1D) | −0.76334 |
| ATP6V1F | ATPase H+ transporting V1 subunit F(ATP6V1F) | −0.12123 |
| ATP6V1H | ATPase H+ transporting V1 subunit H(ATP6V1H) | −0.45183 |
| ATP7A | ATPase copper transporting alpha(ATP7A) | 0.061192 |
| ATP7B | ATPase copper transporting beta(ATP7B) | −0.30403 |
| ATP8A1 | ATPase phospholipid transporting 8A1(ATP8A1) | −0.44102 |
| ATP8A2 | ATPase phospholipid transporting 8A2(ATP8A2) | −0.38336 |
| ATP8B1 | ATPase phospholipid transporting 8B1(ATP8B1) | −0.18356 |
| ATP8B2 | ATPase phospholipid transporting 8B2(ATP8B2) | −0.42023 |
| ATP9A | ATPase phospholipid transporting 9A (putative)(ATP9A) | −0.48155 |
| ATPAF1 | ATP synthase mitochondrial F1 complex assembly factor 1(ATPAF1) | −0.3556 |
| ATPAF2 | ATP synthase mitochondrial F1 complex assembly factor 2(ATPAF2) | −0.54327 |
| AUH | AU RNA binding methylglutaconyl-CoA hydratase(AUH) | −0.40569 |
| B3GNT3 | UDP-GlcNAc:betaGal beta-1,3-N-acetylglucosaminyltransferase 3(B3GNT3) | −0.38929 |
| B4GALNT1 | beta-1,4-N-acetyl-galactosaminyltransferase 1(B4GALNT1) | −0.33839 |
| B4GALT1 | beta-1,4-galactosyltransferase 1(B4GALT1) | −0.46679 |
| B4GALT7 | beta-1,4-galactosyltransferase 7(B4GALT7) | −0.33373 |
| BAAT | bile acid-CoA:amino acid N-acyltransferase(BAAT) | 0.107787 |
| BBOX1 | gamma-butyrobetaine hydroxylase 1(BBOX1) | −0.34856 |
| BCAT1 | branched chain amino acid transaminase 1(BCAT1) | −0.30299 |
| BCAT2 | branched chain amino acid transaminase 2(BCAT2) | −0.17441 |
| BCHE | butyrylcholinesterase(BCHE) | −0.25407 |
| BCKDHA | branched chain keto acid dehydrogenase E1, alpha polypeptide(BCKDHA) | −0.27394 |
| BCKDHB | branched chain keto acid dehydrogenase E1 subunit beta(BCKDHB) | 0.032229 |
| BDH1 | 3-hydroxybutyrate dehydrogenase, type 1(BDH1) | −0.38136 |
| BDH2 | 3-hydroxybutyrate dehydrogenase, type 2(BDH2) | −0.32345 |
| BHMT | betaine--homocysteine S-methyltransferase(BHMT) | 0.025801 |
| BLVRA | biliverdin reductase A(BLVRA) | −0.144 |
| BLVRB | biliverdin reductase B(BLVRB) | −0.23377 |
| BPGM | bisphosphoglycerate mutase(BPGM) | −0.27857 |
| BPHL | biphenyl hydrolase like(BPHL) | −0.28562 |
| BPNT1 | 3′(2′),5′-bisphosphate nucleotidase 1(BPNT1) | −0.09218 |
| BRD8 | bromodomain containing 8(BRD8) | −0.87308 |
| BSND | barttin CLCNK type accessory beta subunit(BSND) | 0.263253 |
| BTD | biotinidase(BTD) | −0.12568 |
| C1orf85 | | −0.15289 |
| CA1 | carbonic anhydrase 1(CA1) | −0.3151 |
| CA10 | carbonic anhydrase 10(CA10) | −0.32017 |
| CA11 | carbonic anhydrase 11(CA11) | −0.36274 |
| CA12 | carbonic anhydrase 12(CA12) | −0.22728 |
| CA13 | carbonic anhydrase 13(CA13) | −0.1299 |
| CA14 | carbonic anhydrase 14(CA14) | −0.42426 |
| CA2 | carbonic anhydrase 2(CA2) | −0.31586 |
| CA3 | carbonic anhydrase 3(CA3) | −0.04721 |
| CA4 | carbonic anhydrase 4(CA4) | −0.12413 |
| CA5A | carbonic anhydrase 5A(CA5A) | −0.20153 |
| CA5B | carbonic anhydrase 5B(CA5B) | −0.04218 |
| CA6 | carbonic anhydrase 6(CA6) | −0.2977 |
| CA7 | carbonic anhydrase 7(CA7) | −0.38786 |
| CA8 | carbonic anhydrase 8(CA8) | −0.69377 |
| CA9 | carbonic anhydrase 9(CA9) | 0.218835 |
| CACNA1A | calcium voltage-gated channel subunit alpha1 A(CACNA1A) | −0.2363 |
| CACNA1B | calcium voltage-gated channel subunit alpha1 B(CACNA1B) | −0.16357 |
| CACNA1C | calcium voltage-gated channel subunit alpha1 C(CACNA1C) | 0.21006 |
| CACNA1D | calcium voltage-gated channel subunit alpha1 D(CACNA1D) | −0.1663 |
| CACNA1E | calcium voltage-gated channel subunit alpha1 E(CACNA1E) | −0.06306 |
| CACNA1F | calcium voltage-gated channel subunit alpha1 F(CACNA1F) | −0.04599 |
| CACNA1G | calcium voltage-gated channel subunit alpha1 G(CACNA1G) | −0.11754 |
| CACNA1H | calcium voltage-gated channel subunit alpha1 H(CACNA1H) | 0.029975 |
| CACNA1I | calcium voltage-gated channel subunit alpha1 I(CACNA1I) | 0.067234 |
| CACNA1S | calcium voltage-gated channel subunit alpha1 S(CACNA1S) | −0.22422 |
| CACNA2D1 | calcium voltage-gated channel auxiliary subunit alpha2delta 1(CACNA2D1) | 0.035365 |
| CACNA2D2 | calcium voltage-gated channel auxiliary subunit alpha2delta 2(CACNA2D2) | −0.20456 |
| CACNA2D3 | calcium voltage-gated channel auxiliary subunit alpha2delta 3(CACNA2D3) | −0.47896 |
| CACNA2D4 | calcium voltage-gated channel auxiliary subunit alpha2delta 4(CACNA2D4) | −0.20228 |
| CACNB1 | calcium voltage-gated channel auxiliary subunit beta 1(CACNB1) | 0.150495 |
| CACNB2 | calcium voltage-gated channel auxiliary subunit beta 2(CACNB2) | −0.51985 |
| CACNB3 | calcium voltage-gated channel auxiliary subunit beta 3(CACNB3) | −0.28831 |
| CACNB4 | calcium voltage-gated channel auxiliary subunit beta 4(CACNB4) | −0.27979 |
| CACNG1 | calcium voltage-gated channel auxiliary subunit gamma 1(CACNG1) | 0.01352 |
| CACNG2 | calcium voltage-gated channel auxiliary subunit gamma 2(CACNG2) | 0.022299 |
| CACNG3 | calcium voltage-gated channel auxiliary subunit gamma 3(CACNG3) | 0.044938 |
| CACNG4 | calcium voltage-gated channel auxiliary subunit gamma 4(CACNG4) | 0.023123 |
| CACNG5 | calcium voltage-gated channel auxiliary subunit gamma 5(CACNG5) | −0.22098 |
| CACNG6 | calcium voltage-gated channel auxiliary subunit gamma 6(CACNG6) | −0.15193 |
| CACNG7 | calcium voltage-gated channel auxiliary subunit gamma 7(CACNG7) | −0.28984 |
| CACNG8 | calcium voltage-gated channel auxiliary subunit gamma 8(CACNG8) | −0.23933 |
| CAD | carbamoyl-phosphate synthetase 2, aspartate transcarbamylase, and dihydro [symbol] | −0.56241 |
| CAMK2G | calcium/calmodulin dependent protein kinase II gamma(CAMK2G) | −0.0007 |
| CAT | catalase(CAT) | −0.07717 |
| CATSPER1 | cation channel sperm associated 1(CATSPER1) | −0.4286 |
| CATSPER2 | cation channel sperm associated 2(CATSPER2) | −0.30937 |
| CATSPER3 | cation channel sperm associated 3(CATSPER3) | −0.2456 |
| CBR1 | carbonyl reductase 1(CBR1) | −0.14147 |
| CBR3 | carbonyl reductase 3(CBR3) | −0.14856 |
| CBR4 | carbonyl reductase 4(CBR4) | −0.34874 |
| CBS | cystathionine-beta-synthase(CBS) | −0.33551 |
| CCBL1 | | −0.23616 |
| CCBL2 | | −0.00176 |
| CCDC102B | coiled-coil domain containing 102B(CCDC102B) | −0.31865 |
| CCDC155 | coiled-coil domain containing 155(CCDC155) | −0.12804 |
| CDA | cytidine deaminase(CDA) | −0.36779 |
| CDADC1 | cytidine and dCMP deaminase domain containing 1(CDADC1) | −0.18899 |
| CDC14A | cell division cycle 14A(CDC14A) | −0.52692 |
| CDC14B | cell division cycle 14B(CDC14B) | −0.33122 |
| CDC25A | cell division cycle 25A(CDC25A) | −0.31252 |
| CDC25B | cell division cycle 25B(CDC25B) | −0.57694 |
| CDC25C | cell division cycle 25C(CDC25C) | 0.02041 |
| CDKN3 | cyclin dependent kinase inhibitor 3(CDKN3) | −0.2304 |
| CDO1 | cysteine dioxygenase type 1(CDO1) | −0.36261 |
| CECR1 | cat eye syndrome chromosome region, candidate 1(CECR1) | 0.137842 |
| CEL | carboxyl ester lipase(CEL) | −0.26263 |
| CERK | ceramide kinase(CERK) | −0.20674 |
| CERKL | ceramide kinase like(CERKL) | −0.47546 |
| CERS1 | ceramide synthase 1(CERS1) | −0.5675 |
| CERS2 | ceramide synthase 2(CERS2) | −0.1757 |
| CERS3 | ceramide synthase 3(CERS3) | −0.39658 |
| CERS4 | ceramide synthase 4(CERS4) | −0.0552 |
| CERS5 | ceramide synthase 5(CERS5) | −0.09954 |
| CERS6 | ceramide synthase 6(CERS6) | −0.23159 |
| CES1 | carboxylesterase 1(CES1) | −0.11025 |
| CES2 | carboxylesterase 2(CES2) | −0.11499 |
| CES3 | carboxylesterase 3(CES3) | −0.18163 |
| CFTR | cystic fibrosis transmembrane conductance regulator(CFTR) | −0.55195 |
| CHAT | choline O-acetyltransferase(CHAT) | 0.038893 |
| CHI3L1 | chitinase 3 like 1(CHI3L1) | −0.08904 |
| CHI3L2 | chitinase 3 like 2(CHI3L2) | −0.20303 |
| CHIA | chitinase, acidic(CHIA) | −0.18538 |
| CHID1 | chitinase domain containing 1(CHID1) | −0.23046 |
| CHIT1 | chitinase 1(CHIT1) | −0.10131 |
| CHKA | choline kinase alpha(CHKA) | 0.189907 |
| CHPF | chondroitin polymerizing factor(CHPF) | −0.11961 |
| CHPT1 | choline phosphotransferase 1(CHPT1) | 0.056641 |
| CHRNA1 | cholinergic receptor nicotinic alpha 1 subunit(CHRNA1) | −0.20911 |
| CHRNA10 | cholinergic receptor nicotinic alpha 10 subunit(CHRNA10) | −0.09517 |
| CHRNA2 | cholinergic receptor nicotinic alpha 2 subunit(CHRNA2) | −0.09543 |
| CHRNA3 | cholinergic receptor nicotinic alpha 3 subunit(CHRNA3) | 0.19304 |
| CHRNA4 | cholinergic receptor nicotinic alpha 4 subunit(CHRNA4) | −0.21187 |
| CHRNA5 | cholinergic receptor nicotinic alpha 5 subunit(CHRNA5) | −0.19271 |
| CHRNA6 | cholinergic receptor nicotinic alpha 6 subunit(CHRNA6) | −0.00193 |
| CHRNA7 | cholinergic receptor nicotinic alpha 7 subunit(CHRNA7) | −0.41657 |
| CHRNA9 | cholinergic receptor nicotinic alpha 9 subunit(CHRNA9) | −0.25425 |
| CHRNB1 | cholinergic receptor nicotinic beta 1 subunit(CHRNB1) | −0.07599 |
| CHRNB2 | cholinergic receptor nicotinic beta 2 subunit(CHRNB2) | −0.22449 |
| CHRNB3 | cholinergic receptor nicotinic beta 3 subunit(CHRNB3) | −0.31891 |
| CHRNB4 | cholinergic receptor nicotinic beta 4 subunit(CHRNB4) | −0.41413 |
| CHRND | cholinergic receptor nicotinic delta subunit(CHRND) | −0.56864 |
| CHRNE | cholinergic receptor nicotinic epsilon subunit(CHRNE) | −0.13696 |
| CHRNG | cholinergic receptor nicotinic gamma subunit(CHRNG) | 0.052055 |
| CHST1 | carbohydrate sulfotransferase 1(CHST1) | −0.0245 |
| CHST6 | carbohydrate sulfotransferase 6(CHST6) | −0.30906 |
| CKB | creatine kinase B(CKB) | −0.24703 |
| CKM | creatine kinase, M-type(CKM) | −0.31264 |
| CKMT2 | creatine kinase, mitochondrial 2(CKMT2) | −0.25249 |
| CLC | Charcot-Leyden crystal galectin(CLC) | −0.31396 |
| CLCA1 | chloride channel accessory 1(CLCA1) | −0.14204 |
| CLCA2 | chloride channel accessory 2(CLCA2) | −0.46141 |
| CLCA4 | chloride channel accessory 4(CLCA4) | 0.053964 |
| CLCN1 | chloride voltage-gated channel 1(CLCN1) | −0.13258 |
| CLCN2 | chloride voltage-gated channel 2(CLCN2) | −0.17466 |
| CLCN3 | chloride voltage-gated channel 3(CLCN3) | −0.49403 |
| CLCN4 | chloride voltage-gated channel 4(CLCN4) | 0.042799 |
| CLCN5 | chloride voltage-gated channel 5(CLCN5) | −0.4161 |
| CLCN6 | chloride voltage-gated channel 6(CLCN6) | −0.36348 |
| CLCN7 | chloride voltage-gated channel 7(CLCN7) | −0.13188 |
| CLCNKA | chloride voltage-gated channel Ka(CLCNKA) | −0.1822 |
| CLCNKB | chloride voltage-gated channel Kb(CLCNKB) | −0.33517 |
| CLIC1 | chloride intracellular channel 1(CLIC1) | −0.09123 |
| CLIC2 | chloride intracellular channel 2(CLIC2) | −0.11586 |
| CLIC3 | chloride intracellular channel 3(CLIC3) | 0.074723 |
| CLIC4 | chloride intracellular channel 4(CLIC4) | −0.06827 |
| CLIC5 | chloride intracellular channel 5(CLIC5) | −0.39353 |
| CLIC6 | chloride intracellular channel 6(CLIC6) | −0.02678 |
| CLYBL | citrate lyase beta like(CLYBL) | 0.057336 |
| CMAS | cytidine monophosphate N-acetylneuraminic acid synthetase(CMAS) | −0.30565 |
| CMPK1 | cytidine/uridine monophosphate kinase 1(CMPK1) | −0.14828 |
| CNDP1 | carnosine dipeptidase 1(CNDP1) | −0.13139 |
| CNDP2 | CNDP dipeptidase 2 (metallopeptidase M20 family)(CNDP2) | −0.17429 |
| CNGA1 | cyclic nucleotide gated channel alpha 1(CNGA1) | −0.18174 |
| CNGA2 | cyclic nucleotide gated channel alpha 2(CNGA2) | −0.35425 |
| CNGA3 | cyclic nucleotide gated channel alpha 3(CNGA3) | 0.054842 |
| CNGA4 | cyclic nucleotide gated channel alpha 4(CNGA4) | −0.3821 |
| CNGB1 | cyclic nucleotide gated channel beta 1(CNGB1) | −0.41649 |
| CNGB3 | cyclic nucleotide gated channel beta 3(CNGB3) | −0.19684 |
| COA5 | cytochrome c oxidase assembly factor 5(COA5) | −0.27193 |
| COASY | Coenzyme A synthase(COASY) | 0.068043 |
| COMT | catechol-O-methyltransferase(COMT) | −0.23521 |
| COMTD1 | catechol-O-methyltransferase domain containing 1(COMTD1) | −0.12303 |
| COQ3 | coenzyme Q3, methyltransferase(COQ3) | −0.11155 |
| COQ5 | coenzyme Q5, methyltransferase(COQ5) | −0.3619 |
| COQ6 | coenzyme Q6, monooxygenase(COQ6) | −0.13567 |
| COQ9 | coenzyme Q9(COQ9) | 0.009879 |
| COX10 | COX10, heme A:farnesyltransferase cytochrome c oxidase assembly factor(CC [symbol] | −0.32329 |
| COX11 | COX11, cytochrome c oxidase copper chaperone(COX11) | −0.31484 |
| COX15 | COX15, cytochrome c oxidase assembly homolog(COX15) | −0.62464 |
| COX17 | COX17, cytochrome c oxidase copper chaperone(COX17) | −0.65998 |
| COX20 | COX20, cytochrome c oxidase assembly factor(COX20) | −0.21636 |
| COX5A | cytochrome c oxidase subunit 5A(COX5A) | −0.09822 |
| COX6B2 | cytochrome c oxidase subunit 6B2(COX6B2) | −0.0858 |
| COX7A1 | cytochrome c oxidase subunit 7A1(COX7A1) | −0.18063 |
| COX7A2L | cytochrome c oxidase subunit 7A2 like(COX7A2L) | −0.33759 |
| CP | ceruloplasmin(CP) | −0.34815 |
| CPOX | coproporphyrinogen oxidase(CPOX) | 0.153277 |
| CPS1 | carbamoyl-phosphate synthase 1(CPS1) | 0.11814 |
| CPT1A | carnitine palmitoyltransferase 1A(CPT1A) | −0.21008 |
| CPT1B | carnitine palmitoyltransferase 1B(CPT1B) | −0.32316 |
| CPT1C | carnitine palmitoyltransferase 1C(CPT1C) | −0.02283 |
| CPT2 | carnitine palmitoyltransferase 2(CPT2) | 0.123329 |
| CRAT | carnitine O-acetyltransferase(CRAT) | −0.11972 |
| CROT | carnitine O-octanoyltransferase(CROT) | −0.25738 |
| CRYL1 | crystallin lambda 1(CRYL1) | −0.42026 |
| CRYM | crystallin mu(CRYM) | 0.088907 |
| CRYZ | crystallin zeta(CRYZ) | −0.67118 |
| CRYZL1 | crystallin zeta like 1(CRYZL1) | −0.50773 |
| CS | citrate synthase(CS) | 0.173973 |
| CSAD | cysteine sulfinic acid decarboxylase(CSAD) | −0.21256 |
| CTDNEP1 | CTD nuclear envelope phosphatase 1(CTDNEP1) | −0.18006 |
| CTDP1 | CTD phosphatase subunit 1(CTDP1) | −0.31504 |
| CTDSP1 | CTD small phosphatase 1(CTDSP1) | −0.06943 |
| CTDSP2 | CTD small phosphatase 2(CTDSP2) | −0.17301 |
| CTDSPL | CTD small phosphatase like(CTDSPL) | −0.23051 |
| CTDSPL2 | CTD small phosphatase like 2(CTDSPL2) | −0.72345 |
| CTH | cystathionine gamma-lyase(CTH) | −0.27029 |
| CTNS | cystinosin, lysosomal cystine transporter(CTNS) | −0.40406 |
| CTPS1 | CTP synthase 1(CTPS1) | −0.36869 |
| CTPS2 | CTP synthase 2(CTPS2) | −0.13748 |
| CUBN | cubilin(CUBN) | −0.03137 |
| CYB5D2 | cytochrome b5 domain containing 2(CYB5D2) | −0.93303 |
| CYB5R1 | cytochrome b5 reductase 1(CYB5R1) | −0.17754 |
| CYB5R2 | cytochrome b5 reductase 2(CYB5R2) | −0.41689 |
| CYB5R3 | cytochrome b5 reductase 3(CYB5R3) | 0.062975 |
| CYB5R4 | cytochrome b5 reductase 4(CYB5R4) | −0.21633 |
| CYB5RL | cytochrome b5 reductase like(CYB5RL) | −0.06717 |
| CYBA | cytochrome b-245 alpha chain(CYBA) | −0.33829 |
| CYBB | cytochrome b-245 beta chain(CYBB) | −0.02661 |
| CYC1 | cytochrome c1(CYC1) | −0.01027 |
| CYCS | cytochrome c, somatic(CYCS) | −0.04523 |
| CYP11A1 | cytochrome P450 family 11 subfamily A member 1(CYP11A1) | −0.1162 |
| CYP11B1 | cytochrome P450 family 11 subfamily B member 1(CYP11B1) | −0.37132 |
| CYP11B2 | cytochrome P450 family 11 subfamily B member 2(CYP11B2) | 0.019282 |
| CYP17A1 | cytochrome P450 family 17 subfamily A member 1(CYP17A1) | −0.2997 |
| CYP19A1 | cytochrome P450 family 19 subfamily A member 1(CYP19A1) | 0.046264 |
| CYP1A1 | cytochrome P450 family 1 subfamily A member 1(CYP1A1) | 0.028119 |
| CYP1A2 | cytochrome P450 family 1 subfamily A member 2(CYP1A2) | −0.06118 |
| CYP1B1 | cytochrome P450 family 1 subfamily B member 1(CYP1B1) | 0.013797 |
| CYP20A1 | cytochrome P450 family 20 subfamily A member 1(CYP20A1) | −0.27375 |
| CYP21A2 | cytochrome P450 family 21 subfamily A member 2(CYP21A2) | −0.0465 |
| CYP24A1 | cytochrome P450 family 24 subfamily A member 1(CYP24A1) | −0.20783 |
| CYP26A1 | cytochrome P450 family 26 subfamily A member 1(CYP26A1) | −0.11988 |
| CYP26B1 | cytochrome P450 family 26 subfamily B member 1(CYP26B1) | 0.036637 |
| CYP26C1 | cytochrome P450 family 26 subfamily C member 1(CYP26C1) | −0.09507 |
| CYP27A1 | cytochrome P450 family 27 subfamily A member 1(CYP27A1) | −0.44032 |
| CYP27B1 | cytochrome P450 family 27 subfamily B member 1(CYP27B1) | 0.015371 |
| CYP27C1 | cytochrome P450 family 27 subfamily C member 1(CYP27C1) | 0.102931 |
| CYP2A13 | cytochrome P450 family 2 subfamily A member 13(CYP2A13) | −0.12234 |
| CYP2A6 | cytochrome P450 family 2 subfamily A member 6(CYP2A6) | −0.13316 |
| CYP2A7 | cytochrome P450 family 2 subfamily A member 7(CYP2A7) | −0.02701 |
| CYP2B6 | cytochrome P450 family 2 subfamily B member 6(CYP2B6) | −0.02414 |
| CYP2C18 | cytochrome P450 family 2 subfamily C member 18(CYP2C18) | −0.36635 |
| CYP2C19 | cytochrome P450 family 2 subfamily C member 19(CYP2C19) | −0.2257 |
| CYP2C8 | cytochrome P450 family 2 subfamily C member 8(CYP2C8) | −0.2513 |
| CYP2C9 | cytochrome P450 family 2 subfamily C member 9(CYP2C9) | −0.44223 |
| CYP2D6 | cytochrome P450 family 2 subfamily D member 6(CYP2D6) | −0.24774 |
| CYP2E1 | cytochrome P450 family 2 subfamily E member 1(CYP2E1) | −0.01693 |
| CYP2F1 | cytochrome P450 family 2 subfamily F member 1(CYP2F1) | −0.21076 |
| CYP2J2 | cytochrome P450 family 2 subfamily J member 2(CYP2J2) | −0.04197 |
| CYP2R1 | cytochrome P450 family 2 subfamily R member 1(CYP2R1) | −0.54822 |
| CYP2S1 | cytochrome P450 family 2 subfamily S member 1(CYP2S1) | −0.43844 |
| CYP2U1 | cytochrome P450 family 2 subfamily U member 1(CYP2U1) | −0.10148 |
| CYP2W1 | cytochrome P450 family 2 subfamily W member 1(CYP2W1) | −0.2905 |
| CYP39A1 | cytochrome P450 family 39 subfamily A member 1(CYP39A1) | −0.20733 |
| CYP3A4 | cytochrome P450 family 3 subfamily A member 4(CYP3A4) | −0.04294 |
| CYP3A43 | cytochrome P450 family 3 subfamily A member 43(CYP3A43) | −0.23968 |
| CYP3A5 | cytochrome P450 family 3 subfamily A member 5(CYP3A5) | −0.06709 |
| CYP3A7 | cytochrome P450 family 3 subfamily A member 7(CYP3A7) | −0.13398 |
| CYP46A1 | cytochrome P450 family 46 subfamily A member 1(CYP46A1) | −0.24459 |
| CYP4A11 | cytochrome P450 family 4 subfamily A member 11(CYP4A11) | −0.16295 |
| CYP4A22 | cytochrome P450 family 4 subfamily A member 22(CYP4A22) | −0.08654 |
| CYP4B1 | cytochrome P450 family 4 subfamily B member 1(CYP4B1) | −0.13854 |
| CYP4F11 | cytochrome P450 family 4 subfamily F member 11(CYP4F11) | −0.28463 |
| CYP4F12 | cytochrome P450 family 4 subfamily F member 12(CYP4F12) | −0.12046 |
| CYP4F2 | cytochrome P450 family 4 subfamily F member 2(CYP4F2) | −0.59303 |
| CYP4F22 | cytochrome P450 family 4 subfamily F member 22(CYP4F22) | −0.57831 |
| CYP4F3 | cytochrome P450 family 4 subfamily F member 3(CYP4F3) | −0.43212 |
| CYP4V2 | cytochrome P450 family 4 subfamily V member 2(CYP4V2) | −0.1495 |
| CYP4X1 | cytochrome P450 family 4 subfamily X member 1(CYP4X1) | 0.4149 |
| CYP4Z1 | cytochrome P450 family 4 subfamily Z member 1(CYP4Z1) | −0.41021 |
| CYP51A1 | cytochrome P450 family 51 subfamily A member 1(CYP51A1) | −0.08749 |
| CYP7A1 | cytochrome P450 family 7 subfamily A member 1(CYP7A1) | −0.03838 |
| CYP7B1 | cytochrome P450 family 7 subfamily B member 1(CYP7B1) | −0.07034 |
| CYP8B1 | cytochrome P450 family 8 subfamily B member 1(CYP8B1) | −0.18946 |
| D2HGDH | D-2-hydroxyglutarate dehydrogenase(D2HGDH) | −0.26507 |
| DAD1 | defender against cell death 1(DAD1) | 0.43074 |
| DAK | | 0.003688 |
| DAO | D-amino acid oxidase(DAO) | −0.49217 |
| DAPP1 | dual adaptor of phosphotyrosine and 3-phosphoinositides 1(DAPP1) | −0.41421 |
| DBH | dopamine beta-hydroxylase(DBH) | −0.17134 |
| DBT | dihydrolipoamide branched chain transacylase E2(DBT) | −0.35636 |
| DCK | deoxycytidine kinase(DCK) | −0.01302 |
| DCT | dopachrome tautomerase(DCT) | 0.029404 |
| DCTD | dCMP deaminase(DCTD) | −0.23302 |
| DCXR | dicarbonyl and L-xylulose reductase(DCXR) | −0.11732 |
| DDC | dopa decarboxylase(DDC) | −0.11722 |
| DDO | D-aspartate oxidase(DDO) | −0.10869 |
| DECR1 | 2,4-dienoyl-CoA reductase 1, mitochondrial(DECR1) | −0.13667 |
| DECR2 | 2,4-dienoyl-CoA reductase 2, peroxisomal(DECR2) | −0.19985 |
| DEFB129 | defensin beta 129(DEFB129) | −0.06484 |
| DEGS1 | delta 4-desaturase, sphingolipid 1(DEGS1) | −1.29665 |
| DEGS2 | delta 4-desaturase, sphingolipid 2(DEGS2) | 0.168849 |
| DERA | deoxyribose-phosphate aldolase(DERA) | −0.05577 |
| DGAT1 | diacylglycerol O-acyltransferase 1(DGAT1) | −0.19652 |
| DGAT2 | diacylglycerol O-acyltransferase 2(DGAT2) | −0.06327 |
| DGAT2L6 | diacylglycerol O-acyltransferase 2 like 6(DGAT2L6) | −0.05158 |
| DGKA | diacylglycerol kinase alpha(DGKA) | −0.23469 |
| DGKB | diacylglycerol kinase beta(DGKB) | −0.10644 |
| DGKD | diacylglycerol kinase delta(DGKD) | −0.49631 |
| DGKE | diacylglycerol kinase epsilon(DGKE) | 0.207485 |
| DGKG | diacylglycerol kinase gamma(DGKG) | −0.2251 |
| DGKH | diacylglycerol kinase eta(DGKH) | −0.16173 |
| DGKI | diacylglycerol kinase iota(DGKI) | −0.39615 |
| DGKQ | diacylglycerol kinase theta(DGKQ) | −0.08207 |
| DGKZ | diacylglycerol kinase zeta(DGKZ) | −0.08535 |
| DGUOK | deoxyguanosine kinase(DGUOK) | −0.02349 |
| DHCR24 | 24-dehydrocholesterol reductase(DHCR24) | −0.1047 |
| DHCR7 | 7-dehydrocholesterol reductase(DHCR7) | −0.32885 |
| DHFR | dihydrofolate reductase(DHFR) | 0.302482 |
| DHFRL1 | | 0.059268 |
| DHODH | dihydroorotate dehydrogenase (quinone)(DHODH) | −0.08631 |
| DHRS1 | dehydrogenase/reductase 1(DHRS1) | −0.21248 |
| DHRS11 | dehydrogenase/reductase 11(DHRS11) | −0.22475 |
| DHRS12 | dehydrogenase/reductase 12(DHRS12) | −0.09529 |
| DHRS13 | dehydrogenase/reductase 13(DHRS13) | 0.086138 |
| DHRS2 | dehydrogenase/reductase 2(DHRS2) | −0.0717 |
| DHRS3 | dehydrogenase/reductase 3(DHRS3) | −0.23169 |
| DHRS4 | dehydrogenase/reductase 4(DHRS4) | −0.25489 |
| DHRS4L2 | dehydrogenase/reductase 4 like 2(DHRS4L2) | −0.16836 |
| DHRS7 | dehydrogenase/reductase 7(DHRS7) | −0.23623 |
| DHRS7B | dehydrogenase/reductase 7B(DHRS7B) | −0.24105 |
| DHRS7C | dehydrogenase/reductase 7C(DHRS7C) | −0.16851 |
| DHRS9 | dehydrogenase/reductase 9(DHRS9) | 0.41947 |
| DHRSX | dehydrogenase/reductase X-linked(DHRSX) | −0.48562 |
| DHTKD1 | dehydrogenase E1 and transketolase domain containing 1(DHTKD1) | −0.34207 |
| DIO1 | deiodinase, iodothyronine type I(DIO1) | −0.13569 |
| DIO2 | deiodinase, iodothyronine type II(DIO2) | −0.07028 |
| DIO3 | deiodinase, iodothyronine type III(DIO3) | −0.20517 |
| DIRC2 | disrupted in renal carcinoma 2(DIRC2) | −0.32247 |
| DLAT | dihydrolipoamide S-acetyltransferase(DLAT) | −0.09114 |
| DLD | dihydrolipoamide dehydrogenase(DLD) | 0.07957 |
| DLG1 | discs large MAGUK scaffold protein 1(DLG1) | −0.63414 |
| DLGAP5 | DLG associated protein 5(DLGAP5) | −0.2788 |
| DLST | dihydrolipoamide S-succinyltransferase(DLST) | 0.170856 |
| DMGDH | dimethylglycine dehydrogenase(DMGDH) | −0.25744 |
| DNAJC6 | DnaJ heat shock protein family (Hsp40) member C6(DNAJC6) | −0.17327 |
| DOLPP1 | dolichyldiphosphatase 1(DOLPP1) | −0.50287 |
| DPEP1 | dipeptidase 1 (renal)(DPEP1) | −0.28281 |
| DPEP2 | dipeptidase 2(DPEP2) | −0.10866 |
| DPEP3 | dipeptidase 3(DPEP3) | −0.36605 |
| DPM1 | dolichyl-phosphate mannosyltransferase subunit 1, catalytic(DPM1) | −0.55452 |
| DPYD | dihydropyrimidine dehydrogenase(DPYD) | −0.18442 |
| DPYS | dihydropyrimidinase(DPYS) | −0.16107 |
| DPYSL2 | dihydropyrimidinase like 2(DPYSL2) | −0.25404 |
| DPYSL3 | dihydropyrimidinase like 3(DPYSL3) | −0.07222 |
| DPYSL4 | dihydropyrimidinase like 4(DPYSL4) | 0.259786 |
| DPYSL5 | dihydropyrimidinase like 5(DPYSL5) | −0.08652 |
| DTYMK | deoxythymidylate kinase(DTYMK) | −0.37698 |
| DUOX1 | dual oxidase 1(DUOX1) | 0.074415 |
| DUOX2 | dual oxidase 2(DUOX2) | −0.31365 |
| DUPD1 | dual specificity phosphatase and pro isomerase domain containing 1(DUPD1) | 0.077704 |
| DUSP1 | dual specificity phosphatase 1(DUSP1) | −0.12388 |
| DUSP10 | dual specificity phosphatase 10(DUSP10) | −0.61078 |
| DUSP11 | dual specificity phosphatase 11(DUSP11) | −0.05079 |
| DUSP12 | dual specificity phosphatase 12(DUSP12) | −0.24482 |
| DUSP13 | dual specificity phosphatase 13(DUSP13) | −0.22521 |
| DUSP14 | dual specificity phosphatase 14(DUSP14) | −0.20941 |
| DUSP15 | dual specificity phosphatase 15(DUSP15) | −0.22642 |
| DUSP16 | dual specificity phosphatase 16(DUSP16) | −0.37902 |
| DUSP18 | dual specificity phosphatase 18(DUSP18) | −0.11007 |
| DUSP19 | dual specificity phosphatase 19(DUSP19) | −0.08059 |
| DUSP2 | dual specificity phosphatase 2(DUSP2) | −0.33922 |
| DUSP21 | dual specificity phosphatase 21(DUSP21) | −0.4716 |
| DUSP22 | dual specificity phosphatase 22(DUSP22) | 0.036355 |
| DUSP23 | dual specificity phosphatase 23(DUSP23) | −0.18247 |
| DUSP26 | dual specificity phosphatase 26 (putative)(DUSP26) | 0.026129 |
| DUSP27 | dual specificity phosphatase 27 (putative)(DUSP27) | −0.61028 |
| DUSP28 | dual specificity phosphatase 28(DUSP28) | −0.23683 |
| DUSP3 | dual specificity phosphatase 3(DUSP3) | −0.2171 |
| DUSP4 | dual specificity phosphatase 4(DUSP4) | −0.50194 |
| DUSP5 | dual specificity phosphatase 5(DUSP5) | −0.23262 |
| DUSP6 | dual specificity phosphatase 6(DUSP6) | −0.35823 |
| DUSP7 | dual specificity phosphatase 7(DUSP7) | 0.033278 |
| DUSP8 | dual specificity phosphatase 8(DUSP8) | −0.23542 |
| DUSP9 | dual specificity phosphatase 9(DUSP9) | −0.04255 |
| DUT | deoxyuridine triphosphatase(DUT) | −0.29488 |
| EBP | emopamil binding protein (sterol isomerase)(EBP) | −0.02785 |
| EBPL | emopamil binding protein like(EBPL) | −0.36989 |
| ECH1 | enoyl-CoA hydratase 1(ECH1) | −0.25844 |
| ECHDC1 | ethylmalonyl-CoA decarboxylase 1(ECHDC1) | −0.19007 |
| ECHDC2 | enoyl-CoA hydratase domain containing 2(ECHDC2) | −0.02211 |
| ECHDC3 | enoyl-CoA hydratase domain containing 3(ECHDC3) | −0.19365 |
| ECHS1 | enoyl-CoA hydratase, short chain 1(ECHS1) | 0.0552 |
| ECI1 | enoyl-CoA delta isomerase 1(ECI1) | −0.12468 |
| ECI2 | enoyl-CoA delta isomerase 2(ECI2) | −0.12573 |
| EDEM1 | ER degradation enhancing alpha-mannosidase like protein 1(EDEM1) | −0.11372 |
| EHHADH | enoyl-CoA hydratase and 3-hydroxyacyl CoA dehydrogenase(EHHADH) | −0.28304 |
| ELOVL1 | ELOVL fatty acid elongase 1(ELOVL1) | −0.641 |
| ELOVL2 | ELOVL fatty acid elongase 2(ELOVL2) | −0.07479 |
| ELOVL3 | ELOVL fatty acid elongase 3(ELOVL3) | 0.12463 |
| ELOVL4 | ELOVL fatty acid elongase 4(ELOVL4) | −0.2271 |
| ELOVL5 | ELOVL fatty acid elongase 5(ELOVL5) | −0.1699 |
| ELOVL6 | ELOVL fatty acid elongase 6(ELOVL6) | −0.07665 |
| ELOVL7 | ELOVL fatty acid elongase 7(ELOVL7) | −0.14127 |
| ENO1 | enolase 1(ENO1) | −0.55532 |
| ENO2 | enolase 2(ENO2) | −0.30526 |
| ENO3 | enolase 3(ENO3) | −0.18626 |
| ENPP1 | ectonucleotide pyrophosphatase/phosphodiesterase 1(ENPP1) | −0.14143 |
| ENPP2 | ectonucleotide pyrophosphatase/phosphodiesterase 2(ENPP2) | −0.1586 |
| ENPP3 | ectonucleotide pyrophosphatase/phosphodiesterase 3(ENPP3) | −0.21566 |
| ENPP4 | ectonucleotide pyrophosphatase/phosphodiesterase 4 (putative)(ENPP4) | −0.28155 |
| ENPP5 | ectonucleotide pyrophosphatase/phosphodiesterase 5 (putative)(ENPP5) | −0.35074 |
| ENTHD1 | ENTH domain containing 1(ENTHD1) | 0.161264 |
| ENTPD1 | ectonucleoside triphosphate diphosphohydrolase 1(ENTPD1) | −0.12032 |
| ENTPD3 | ectonucleoside triphosphate diphosphohydrolase 3(ENTPD3) | −0.26166 |
| ENTPD5 | ectonucleoside triphosphate diphosphohydrolase 5(ENTPD5) | −0.42479 |
| ENTPD8 | ectonucleoside triphosphate diphosphohydrolase 8(ENTPD8) | −0.10932 |
| EPHX1 | epoxide hydrolase 1(EPHX1) | −0.22586 |
| EPHX2 | epoxide hydrolase 2(EPHX2) | −0.06149 |
| EPM2A | epilepsy, progressive myoclonus type 2A, Lafora disease (laforin)(EPM2A) | −0.26617 |
| EPX | eosinophil peroxidase(EPX) | 0.053134 |
| ESD | esterase D(ESD) | −0.19367 |
| ESR1 | estrogen receptor 1(ESR1) | −0.24667 |
| ESR2 | estrogen receptor 2(ESR2) | −0.27541 |
| ESRRA | estrogen related receptor alpha(ESRRA) | −0.01016 |
| ESRRB | estrogen related receptor beta(ESRRB) | −0.04631 |
| ESRRG | estrogen related receptor gamma(ESRRG) | −0.13909 |
| ETFA | electron transfer flavoprotein alpha subunit(ETFA) | −0.16858 |
| ETFB | electron transfer flavoprotein beta subunit(ETFB) | −0.35656 |
| ETFDH | electron transfer flavoprotein dehydrogenase(ETFDH) | −0.23092 |
| ETNK1 | ethanolamine kinase 1(ETNK1) | −0.34703 |
| ETNK2 | ethanolamine kinase 2(ETNK2) | −0.14117 |
| EXT1 | exostosin glycosyltransferase 1(EXT1) | −0.53823 |
| EXT2 | exostosin glycosyltransferase 2(EXT2) | −0.12154 |
| EXTL1 | exostosin like glycosyltransferase 1(EXTL1) | −0.12351 |
| EXTL2 | exostosin like glycosyltransferase 2(EXTL2) | −0.33882 |
| EXTL3 | exostosin like glycosyltransferase 3(EXTL3) | −0.23727 |
| EYA1 | EYA transcriptional coactivator and phosphatase 1(EYA1) | −0.49618 |
| EYA2 | EYA transcriptional coactivator and phosphatase 2(EYA2) | 0.075331 |
| EYA3 | EYA transcriptional coactivator and phosphatase 3(EYA3) | −0.16646 |
| EYA4 | EYA transcriptional coactivator and phosphatase 4(EYA4) | −0.12489 |
| FA2H | fatty acid 2-hydroxylase(FA2H) | −0.21476 |
| FAAH | fatty acid amide hydrolase(FAAH) | −0.11011 |
| FAAH2 | fatty acid amide hydrolase 2(FAAH2) | 0.003786 |
| FABP1 | fatty acid binding protein 1(FABP1) | −0.46239 |
| FABP2 | fatty acid binding protein 2(FABP2) | −0.02207 |
| FABP3 | fatty acid binding protein 3(FABP3) | −0.15161 |
| FABP4 | fatty acid binding protein 4(FABP4) | −0.11384 |
| FABP6 | fatty acid binding protein 6(FABP6) | −0.25603 |
| FADS1 | fatty acid desaturase 1(FADS1) | −0.18401 |
| FADS2 | fatty acid desaturase 2(FADS2) | −0.40122 |
| FADS3 | fatty acid desaturase 3(FADS3) | −0.20438 |
| FAH | fumarylacetoacetate hydrolase(FAH) | −0.23307 |
| FAR1 | fatty acyl-CoA reductase 1(FAR1) | 0.037493 |
| FAR2 | fatty acyl-CoA reductase 2(FAR2) | −0.5209 |
| FASN | fatty acid synthase(FASN) | 0.038527 |
| FBP1 | fructose-bisphosphatase 1(FBP1) | 0.047433 |
| FBP2 | fructose-bisphosphatase 2(FBP2) | −0.10024 |
| FDFT1 | farnesyl-diphosphate farnesyltransferase 1(FDFT1) | −0.10062 |
| FDPS | farnesyl diphosphate synthase(FDPS) | −0.32253 |
| FDX1 | ferredoxin 1(FDX1) | −0.08935 |
| FDXR | ferredoxin reductase(FDXR) | 0.198128 |
| FECH | ferrochelatase(FECH) | −0.2876 |
| FH | fumarate hydratase(FH) | 0.103477 |
| FHIT | fragile histidine triad(FHIT) | −0.1716 |
| FLAD1 | flavin adenine dinucleotide synthetase 1(FLAD1) | −0.10015 |
| FLVCR1 | feline leukemia virus subgroup C cellular receptor 1(FLVCR1) | −0.52802 |
| FLVCR2 | feline leukemia virus subgroup C cellular receptor family member 2(FLVCR2) | 0.040283 |
| FMO1 | flavin containing monooxygenase 1(FMO1) | −0.43658 |
| FMO3 | flavin containing monooxygenase 3(FMO3) | 0.059538 |
| FMO4 | flavin containing monooxygenase 4(FMO4) | −0.37695 |
| FMO5 | flavin containing monooxygenase 5(FMO5) | −0.10181 |
| FN3K | fructosamine 3 kinase(FN3K) | −0.13426 |
| FN3KRP | fructosamine 3 kinase related protein(FN3KRP) | −0.14006 |
| FOLH1 | folate hydrolase 1(FOLH1) | −0.19706 |
| FOLR1 | folate receptor 1(FOLR1) | −0.21652 |
| FOLR2 | folate receptor beta(FOLR2) | −0.27299 |
| FPGS | folylpolyglutamate synthase(FPGS) | −0.46161 |
| FPGT | fucose-1-phosphate guanylyltransferase(FPGT) | −0.17188 |
| FRRS1 | ferric chelate reductase 1(FRRS1) | −0.44289 |
| FUCA1 | fucosidase, alpha-L- 1, tissue(FUCA1) | −0.2188 |
| FUCA2 | fucosidase, alpha-L- 2, plasma(FUCA2) | −0.11625 |
| FUK | fucokinase(FUK) | −0.35632 |
| FUT1 | fucosyltransferase 1 (H blood group)(FUT1) | −0.29616 |
| FUT2 | fucosyltransferase 2(FUT2) | 0.02037 |
| FUT3 | fucosyltransferase 3 (Lewis blood group)(FUT3) | 0.27538 |
| FUT6 | fucosyltransferase 6(FUT6) | −0.17568 |
| FUT8 | fucosyltransferase 8(FUT8) | −0.65846 |
| FXN | frataxin(FXN) | 0.24774 |
| G6PC | glucose-6-phosphatase catalytic subunit(G6PC) | −0.22502 |
| G6PC2 | glucose-6-phosphatase catalytic subunit 2(G6PC2) | −0.21184 |
| G6PC3 | glucose-6-phosphatase catalytic subunit 3(G6PC3) | −0.0621 |
| G6PD | glucose-6-phosphate dehydrogenase(G6PD) | −0.10164 |
| GAA | glucosidase alpha, acid(GAA) | 0.054188 |
| GABRA1 | gamma-aminobutyric acid type A receptor alpha1 subunit(GABRA1) | −0.17117 |
| GABRA2 | gamma-aminobutyric acid type A receptor alpha2 subunit(GABRA2) | −0.37505 |
| GABRA3 | gamma-aminobutyric acid type A receptor alpha3 subunit(GABRA3) | 0.011495 |
| GABRA4 | gamma-aminobutyric acid type A receptor alpha4 subunit(GABRA4) | −0.01614 |
| GABRA5 | gamma-aminobutyric acid type A receptor alpha5 subunit(GABRA5) | −0.16378 |
| GABRA6 | gamma-aminobutyric acid type A receptor alpha6 subunit(GABRA6) | −0.30372 |
| GABRB1 | gamma-aminobutyric acid type A receptor beta1 subunit(GABRB1) | −0.30491 |
| GABRB2 | gamma-aminobutyric acid type A receptor beta2 subunit(GABRB2) | −0.31552 |
| GABRB3 | gamma-aminobutyric acid type A receptor beta3 subunit(GABRB3) | 0.10688 |
| GABRD | gamma-aminobutyric acid type A receptor delta subunit(GABRD) | −0.34262 |
| GABRE | gamma-aminobutyric acid type A receptor epsilon subunit(GABRE) | −0.02287 |
| GABRG1 | gamma-aminobutyric acid type A receptor gamma1 subunit(GABRG1) | −0.07527 |
| GABRG2 | gamma-aminobutyric acid type A receptor gamma2 subunit(GABRG2) | −0.00326 |
| GABRG3 | gamma-aminobutyric acid type A receptor gamma3 subunit(GABRG3) | −0.31852 |
| GABRP | gamma-aminobutyric acid type A receptor pi subunit(GABRP) | 0.027419 |
| GABRQ | gamma-aminobutyric acid type A receptor theta subunit(GABRQ) | 0.217229 |
| GABRR1 | gamma-aminobutyric acid type A receptor rho1 subunit(GABRR1) | −0.22456 |
| GABRR2 | gamma-aminobutyric acid type A receptor rho2 subunit(GABRR2) | −0.12418 |
| GAD1 | glutamate decarboxylase 1(GAD1) | 0.016857 |
| GAD2 | glutamate decarboxylase 2(GAD2) | 0.074051 |
| GADL1 | glutamate decarboxylase like 1(GADL1) | −0.35494 |
| GAL3ST1 | galactose-3-O-sulfotransferase 1(GAL3ST1) | −0.17043 |
| GALC | galactosylceramidase(GALC) | −0.52398 |
| GALE | UDP-galactose-4-epimerase(GALE) | −0.4214 |
| GALK1 | galactokinase 1(GALK1) | −0.13658 |
| GALK2 | galactokinase 2(GALK2) | −0.07504 |
| GALM | galactose mutarotase(GALM) | −0.28167 |
| GALNS | galactosamine (N-acetyl)-6-sulfatase(GALNS) | −0.33147 |
| GALT | galactose-1-phosphate uridylyltransferase(GALT) | −0.40476 |
| GAMT | guanidinoacetate N-methyltransferase(GAMT) | −0.13109 |
| GANAB | glucosidase II alpha subunit(GANAB) | 0.23609 |
| GANC | glucosidase alpha, neutral C(GANC) | −0.06012 |
| GAPDH | glyceraldehyde-3-phosphate dehydrogenase(GAPDH) | 0.069527 |
| GAPDHS | glyceraldehyde-3-phosphate dehydrogenase, spermatogenic(GAPDHS) | −0.06205 |
| GART | phosphoribosylglycinamide formyltransferase, phosphoribosylglycinamide sy [symbol] | −0.08482 |
| GATM | glycine amidinotransferase(GATM) | −1.21614 |
| GBA | glucosylceramidase beta(GBA) | −0.38732 |
| GBA2 | glucosylceramidase beta 2(GBA2) | −0.18889 |
| GBE1 | 1,4-alpha-glucan branching enzyme 1(GBE1) | 0.001183 |
| GCAT | glycine C-acetyltransferase(GCAT) | −0.09239 |
| GCDH | glutaryl-CoA dehydrogenase(GCDH) | −0.40835 |
| GCH1 | GTP cyclohydrolase 1(GCH1) | −0.30614 |
| GCK | glucokinase(GCK) | −0.28724 |
| GCLC | glutamate-cysteine ligase catalytic subunit(GCLC) | −0.41608 |
| GCLM | glutamate-cysteine ligase modifier subunit(GCLM) | −0.13782 |
| GCNT2 | glucosaminyl (N-acetyl) transferase 2, I-branching enzyme (I blood group) (GCI [symbol] | −0.19572 |
| GCSH | glycine cleavage system protein H(GCSH) | 0.080723 |
| GDA | guanine deaminase(GDA) | −0.31352 |
| GDPD3 | glycerophosphodiester phosphodiesterase domain containing 3(GDPD3) | −0.01428 |
| GFOD1 | glucose-fructose oxidoreductase domain containing 1(GFOD1) | −0.13132 |
| GFOD2 | glucose-fructose oxidoreductase domain containing 2(GFOD2) | −0.03429 |
| GFPT1 | glutamine--fructose-6-phosphate transaminase 1(GFPT1) | −0.67188 |
| GFPT2 | glutamine-fructose-6-phosphate transaminase 2(GFPT2) | 0.034559 |
| GGH | gamma-glutamyl hydrolase(GGH) | −0.31271 |
| GGPS1 | geranylgeranyl diphosphate synthase 1(GGPS1) | −0.20178 |
| GGT1 | gamma-glutamyltransferase 1(GGT1) | −0.16742 |
| GGT5 | gamma-glutamyltransferase 5(GGT5) | 0.031972 |
| GGT6 | gamma-glutamyltransferase 6(GGT6) | −0.36395 |
| GGT7 | gamma-glutamyltransferase 7(GGT7) | −0.09248 |
| GGTLC1 | gamma-glutamyltransferase light chain 1(GGTLC1) | 0.071615 |
| GGTLC2 | gamma-glutamyltransferase light chain 2(GGTLC2) | −0.05891 |
| GK | glycerol kinase(GK) | −0.15415 |
| GK2 | glycerol kinase 2(GK2) | 0.21754 |
| GLA | galactosidase alpha(GLA) | 0.170268 |
| GLB1 | galactosidase beta 1(GLB1) | −0.23332 |
| GLDC | glycine decarboxylase(GLDC) | 0.002736 |
| GLO1 | glyoxalase I(GLO1) | −0.03561 |
| GLRA1 | glycine receptor alpha 1(GLRA1) | 0.010228 |
| GLRA2 | glycine receptor alpha 2(GLRA2) | −0.54128 |
| GLRA3 | glycine receptor alpha 3(GLRA3) | −0.25378 |
| GLRA4 | glycine receptor alpha 4(GLRA4) | 0.35535 |
| GLRB | glycine receptor beta(GLRB) | −0.05775 |
| GLRX | glutaredoxin(GLRX) | −0.34496 |
| GLRX2 | glutaredoxin 2(GLRX2) | −0.50048 |
| GLRX3 | glutaredoxin 3(GLRX3) | −0.12487 |
| GLS | glutaminase(GLS) | −0.18619 |
| GLS2 | glutaminase 2(GLS2) | −0.11969 |
| GLUD1 | glutamate dehydrogenase 1(GLUD1) | 0.044824 |
| GLUD2 | glutamate dehydrogenase 2(GLUD2) | −0.12899 |
| GLUL | glutamate-ammonia ligase(GLUL) | −0.28521 |
| GLYAT | glycine-N-acyltransferase(GLYAT) | −0.17631 |
| GLYCTK | glycerate kinase(GLYCTK) | −0.13641 |
| GMDS | GDP-mannose 4,6-dehydratase(GMDS) | −0.4645 |
| GMPPA | GDP-mannose pyrophosphorylase A(GMPPA) | 0.12895 |
| GMPPB | GDP-mannose pyrophosphorylase B(GMPPB) | 0.140711 |
| GMPR | guanosine monophosphate reductase(GMPR) | −0.19881 |
| GMPR2 | guanosine monophosphate reductase 2(GMPR2) | −0.1054 |
| GMPS | guanine monophosphate synthase(GMPS) | −0.12914 |
| GNE | glucosamine (UDP-N-acetyl)-2-epimerase/N-acetylmannosamine kinase(GNE) | −0.31243 |
| GNMT | glycine N-methyltransferase(GNMT) | 0.2429 |
| GNPAT | glyceronephosphate O-acyltransferase(GNPAT) | −0.02942 |
| GNPDA1 | glucosamine-6-phosphate deaminase 1(GNPDA1) | −0.16829 |
| GNPDA2 | glucosamine-6-phosphate deaminase 2(GNPDA2) | 0.061421 |
| GNPNAT1 | glucosamine-phosphate N-acetyltransferase 1(GNPNAT1) | −0.12048 |
| GNPTAB | N-acetylglucosamine-1-phosphate transferase alpha and beta subunits(GNPT, [symbol] | −1.07218 |
| GNPTG | N-acetylglucosamine-1-phosphate transferase gamma subunit(GNPTG) | −0.46636 |
| GNS | glucosamine (N-acetyl)-6-sulfatase(GNS) | −0.305 |
| GOT1 | glutamic-oxaloacetic transaminase 1(GOT1) | −0.67637 |
| GOT2 | glutamic-oxaloacetic transaminase 2(GOT2) | −0.15241 |
| GPD1 | glycerol-3-phosphate dehydrogenase 1(GPD1) | −0.04588 |
| GPD2 | glycerol-3-phosphate dehydrogenase 2(GPD2) | −0.07779 |
| GPHN | gephyrin(GPHN) | 0.009412 |
| GPI | glucose-6-phosphate isomerase(GPI) | −0.03267 |
| GPLD1 | glycosylphosphatidylinositol specific phospholipase D1(GPLD1) | −0.13809 |
| GPT | glutamic--pyruvic transaminase(GPT) | 0.31417 |
| GPT2 | glutamic--pyruvic transaminase 2(GPT2) | −0.17828 |
| GPX1 | glutathione peroxidase 1(GPX1) | −0.31198 |
| GPX2 | glutathione peroxidase 2(GPX2) | 0.021441 |
| GPX3 | glutathione peroxidase 3(GPX3) | −0.21012 |
| GPX4 | glutathione peroxidase 4(GPX4) | 0.51225 |
| GPX5 | glutathione peroxidase 5(GPX5) | −0.09298 |
| GPX6 | glutathione peroxidase 6(GPX6) | −0.32009 |
| GPX7 | glutathione peroxidase 7(GPX7) | −0.24405 |
| GPX8 | glutathione peroxidase 8 (putative)(GPX8) | −0.08677 |
| GRHPR | glyoxylate and hydroxypyruvate reductase(GRHPR) | −0.29306 |
| GRIA1 | glutamate ionotropic receptor AMPA type subunit 1(GRIA1) | −0.22011 |
| GRIA2 | glutamate ionotropic receptor AMPA type subunit 2(GRIA2) | −0.15834 |
| GRIA3 | glutamate ionotropic receptor AMPA type subunit 3(GRIA3) | −0.01662 |
| GRIA4 | glutamate ionotropic receptor AMPA type subunit 4(GRIA4) | −0.09529 |
| GRID1 | glutamate ionotropic receptor delta type subunit 1(GRID1) | 0.01401 |
| GRID2 | glutamate ionotropic receptor delta type subunit 2(GRID2) | −0.02345 |
| GRIK1 | glutamate ionotropic receptor kainate type subunit 1(GRIK1) | 0.034904 |
| GRIK2 | glutamate ionotropic receptor kainate type subunit 2(GRIK2) | 0.026043 |
| GRIK3 | glutamate ionotropic receptor kainate type subunit 3(GRIK3) | 0.054636 |
| GRIK4 | glutamate ionotropic receptor kainate type subunit 4(GRIK4) | −0.29963 |
| GRIK5 | glutamate ionotropic receptor kainate type subunit 5(GRIK5) | −0.20822 |
| GRIN1 | glutamate ionotropic receptor NMDA type subunit 1(GRIN1) | −0.10573 |
| GRIN2A | glutamate ionotropic receptor NMDA type subunit 2A(GRIN2A) | −0.10553 |
| GRIN2B | glutamate ionotropic receptor NMDA type subunit 2B(GRIN2B) | −0.1726 |
| GRIN2C | glutamate ionotropic receptor NMDA type subunit 2C(GRIN2C) | −0.06384 |
| GRIN2D | glutamate ionotropic receptor NMDA type subunit 2D(GRIN2D) | −0.74322 |
| GRIN3A | glutamate ionotropic receptor NMDA type subunit 3A(GRIN3A) | −0.15559 |
| GRIN3B | glutamate ionotropic receptor NMDA type subunit 3B(GRIN3B) | 0.029739 |
| GSR | glutathione-disulfide reductase(GSR) | 0.35392 |
| GSS | glutathione synthetase(GSS) | −0.27073 |
| GSTA1 | glutathione S-transferase alpha 1(GSTA1) | −0.11294 |
| GSTA2 | glutathione S-transferase alpha 2(GSTA2) | −0.29052 |
| GSTA3 | glutathione S-transferase alpha 3(GSTA3) | 0.26486 |
| GSTA4 | glutathione S-transferase alpha 4(GSTA4) | 0.30064 |
| GSTA5 | glutathione S-transferase alpha 5(GSTA5) | −0.10121 |
| GSTCD | glutathione S-transferase C-terminal domain containing(GSTCD) | −0.33707 |
| GSTK1 | glutathione S-transferase kappa 1(GSTK1) | 0.110222 |
| GSTM1 | glutathione S-transferase mu 1(GSTM1) | −0.0235 |
| GSTM2 | glutathione S-transferase mu 2(GSTM2) | 0.11731 |
| GSTM3 | glutathione S-transferase mu 3(GSTM3) | −0.06963 |
| GSTM4 | glutathione S-transferase mu 4(GSTM4) | −0.18409 |
| GSTM5 | glutathione S-transferase mu 5(GSTM5) | −0.0802 |
| GSTO1 | glutathione S-transferase omega 1(GSTO1) | 0.007078 |
| GSTO2 | glutathione S-transferase omega 2(GSTO2) | −0.37995 |
| GSTP1 | glutathione S-transferase pi 1(GSTP1) | 0.25099 |
| GSTT1 | glutathione S-transferase theta 1(GSTT1) | −0.19232 |
| GSTT2 | glutathione S-transferase theta 2 (gene/pseudogene)(GSTT2) | 0.138902 |
| GSTZ1 | glutathione S-transferase zeta 1(GSTZ1) | −0.06501 |
| GUCY1A2 | guanylate cyclase 1 soluble subunit alpha 2(GUCY1A2) | 0.07905 |
| GUCY1A3 | guanylate cyclase 1 soluble subunit alpha(GUCY1A3) | −0.32101 |
| GUCY1B3 | guanylate cyclase 1 soluble subunit beta(GUCY1B3) | −0.11633 |
| GUCY2C | guanylate cyclase 2C(GUCY2C) | −0.23148 |
| GUCY2D | guanylate cyclase 2D, retinal(GUCY2D) | −0.15554 |
| GUCY2F | guanylate cyclase 2F, retinal(GUCY2F) | −0.01483 |
| GUK1 | guanylate kinase 1(GUK1) | −0.422 |
| GUSB | glucuronidase beta(GUSB) | −0.0517 |
| GYG1 | glycogenin 1(GYG1) | −0.01542 |
| GYG2 | glycogenin 2(GYG2) | −0.06388 |
| GYS1 | glycogen synthase 1(GYS1) | −0.04046 |
| GYS2 | glycogen synthase 2(GYS2) | −0.18325 |
| H6PD | hexose-6-phosphate dehydrogenase/glucose 1-dehydrogenase(H6PD) | −0.31596 |
| HACL1 | 2-hydroxyacyl-CoA lyase 1(HACL1) | −0.21418 |
| HADH | hydroxyacyl-CoA dehydrogenase(HADH) | −0.15681 |
| HADHA | hydroxyacyl-CoA dehydrogenase/3-ketoacyl-CoA thiolase/enoyl-CoA hydratas [symbol] | −0.30153 |
| HADHB | hydroxyacyl-CoA dehydrogenase/3-ketoacyl-CoA thiolase/enoyl-CoA hydratas [symbol] | −0.04983 |
| HAGH | hydroxyacylglutathione hydrolase(HAGH) | −0.14569 |
| HAGHL | hydroxyacylglutathione hydrolase-like(HAGHL) | −0.27827 |
| HAL | histidine ammonia-lyase(HAL) | 0.29924 |
| HAS1 | hyaluronan synthase 1(HAS1) | 0.026951 |
| HAS2 | hyaluronan synthase 2(HAS2) | 0.079701 |
| HCCS | holocytochrome c synthase(HCCS) | −0.05218 |
| HCN1 | hyperpolarization activated cyclic nucleotide gated potassium channel 1(HCN [symbol] | −0.07574 |
| HCN2 | hyperpolarization activated cyclic nucleotide gated potassium channel 2(HCN [symbol] | −0.60553 |
| HCN3 | hyperpolarization activated cyclic nucleotide gated potassium channel 3(HCN [symbol] | −0.25662 |
| HCN4 | hyperpolarization activated cyclic nucleotide gated potassium channel 4(HCN [symbol] | −0.18044 |
| HDC | histidine decarboxylase(HDC) | −0.01265 |
| HEPH | hephaestin(HEPH) | −0.02643 |
| HEXA | hexosaminidase subunit alpha(HEXA) | −0.00754 |
| HEXB | hexosaminidase subunit beta(HEXB) | −0.21728 |
| HGD | homogentisate 1,2-dioxygenase(HGD) | −0.08008 |
| HIBCH | 3-hydroxyisobutyryl-CoA hydrolase(HIBCH) | −0.05954 |
| HK1 | hexokinase 1(HK1) | −0.40614 |
| HK2 | hexokinase 2(HK2) | −0.10345 |
| HK3 | hexokinase 3(HK3) | −0.2603 |
| HKDC1 | hexokinase domain containing 1(HKDC1) | −0.14663 |
| HLCS | holocarboxylase synthetase(HLCS) | −0.31362 |
| HMBS | hydroxymethylbilane synthase(HMBS) | −0.44127 |
| HMGCL | 3-hydroxymethyl-3-methylglutaryl-CoA lyase(HMGCL) | −0.1133 |
| HMGCR | 3-hydroxy-3-methylglutaryl-CoA reductase(HMGCR) | −1.03835 |
| HMGCS1 | 3-hydroxy-3-methylglutaryl-CoA synthase 1(HMGCS1) | −0.23214 |
| HMGCS2 | 3-hydroxy-3-methylglutaryl-CoA synthase 2(HMGCS2) | −0.23888 |
| HMOX1 | heme oxygenase 1(HMOX1) | −0.1565 |
| HMOX2 | heme oxygenase 2(HMOX2) | 0.015743 |
| HMX1 | H6 family homeobox 1(HMX1) | 0.062777 |
| HNF4A | hepatocyte nuclear factor 4 alpha(HNF4A) | 0.015145 |
| HNF4G | hepatocyte nuclear factor 4 gamma(HNF4G) | −0.80308 |
| HNMT | histamine N-methyltransferase(HNMT) | −0.25897 |
| HPD | 4-hydroxyphenylpyruvate dioxygenase(HPD) | 0.022555 |
| HPGD | hydroxyprostaglandin dehydrogenase 15-(NAD)(HPGD) | −0.17152 |
| HPGDS | hematopoietic prostaglandin D synthase(HPGDS) | −0.1239 |
| HPRT1 | hypoxanthine phosphoribosyltransferase 1(HPRT1) | −0.34191 |
| HS2ST1 | heparan sulfate 2-O-sulfotransferase 1(HS2ST1) | −0.31959 |
| HS3ST1 | heparan sulfate-glucosamine 3-sulfotransferase 1(HS3ST1) | −0.349 |
| HSD11B1 | hydroxysteroid 11-beta dehydrogenase 1(HSD11B1) | −0.47948 |
| HSD11B1L | hydroxysteroid 11-beta dehydrogenase 1 like(HSD11B1L) | 0.282928 |
| HSD11B2 | hydroxysteroid 11-beta dehydrogenase 2(HSD11B2) | −0.16095 |
| HSD17B1 | hydroxysteroid 17-beta dehydrogenase 1(HSD17B1) | −0.2692 |
| HSD17B10 | hydroxysteroid 17-beta dehydrogenase 10(HSD17B10) | −0.72504 |
| HSD17B11 | hydroxysteroid 17-beta dehydrogenase 11(HSD17B11) | −0.05592 |
| HSD17B12 | hydroxysteroid 17-beta dehydrogenase 12(HSD17B12) | −0.41374 |
| HSD17B13 | hydroxysteroid 17-beta dehydrogenase 13(HSD17B13) | −0.24457 |
| HSD17B14 | hydroxysteroid 17-beta dehydrogenase 14(HSD17B14) | −0.18894 |
| HSD17B2 | hydroxysteroid 17-beta dehydrogenase 2(HSD17B2) | −0.08844 |
| HSD17B3 | hydroxysteroid 17-beta dehydrogenase 3(HSD17B3) | −0.05522 |
| HSD17B4 | hydroxysteroid 17-beta dehydrogenase 4(HSD17B4) | −0.11062 |
| HSD17B6 | hydroxysteroid 17-beta dehydrogenase 6(HSD17B6) | −0.33772 |
| HSD17B7 | hydroxysteroid 17-beta dehydrogenase 7(HSD17B7) | −0.3151 |
| HSD17B8 | hydroxysteroid 17-beta dehydrogenase 8(HSD17B8) | −0.04725 |
| HSD3B1 | hydroxy-delta-5-steroid dehydrogenase, 3 beta- and steroid delta-isomerase [symbol] | −0.20769 |
| HSD3B2 | hydroxy-delta-5-steroid dehydrogenase, 3 beta- and steroid delta-isomerase [symbol] | 0.034969 |
| HSD3B7 | hydroxy-delta-5-steroid dehydrogenase, 3 beta- and steroid delta-isomerase [symbol] | −0.23389 |
| HSDL1 | hydroxysteroid dehydrogenase like 1(HSDL1) | −0.36347 |
| HSDL2 | hydroxysteroid dehydrogenase like 2(HSDL2) | −0.19136 |
| HTR3A | 5-hydroxytryptamine receptor 3A(HTR3A) | −0.26015 |
| HTR3B | 5-hydroxytryptamine receptor 3B(HTR3B) | 0.058299 |
| HTR3C | 5-hydroxytryptamine receptor 3C(HTR3C) | −0.20876 |
| HTR3D | 5-hydroxytryptamine receptor 3D(HTR3D) | −0.01942 |
| HTR3E | 5-hydroxytryptamine receptor 3E(HTR3E) | −0.30173 |
| HVCN1 | hydrogen voltage gated channel 1(HVCN1) | −0.19725 |
| HYAL1 | hyaluronoglucosaminidase 1(HYAL1) | 0.02038 |
| HYAL2 | hyaluronoglucosaminidase 2(HYAL2) | −0.02666 |
| HYI | hydroxypyruvate isomerase (putative)(HYI) | −0.37654 |
| IAPP | islet amyloid polypeptide(IAPP) | −0.24557 |
| IDH1 | isocitrate dehydrogenase (NADP(+)) 1, cytosolic(IDH1) | −0.23607 |
| IDH2 | isocitrate dehydrogenase (NADP(+)) 2, mitochondrial(IDH2) | −0.22581 |
| IDH3A | isocitrate dehydrogenase 3 (NAD(+)) alpha(IDH3A) | −0.11323 |
| IDH3B | isocitrate dehydrogenase 3 (NAD(+)) beta(IDH3B) | −0.48407 |
| IDH3G | isocitrate dehydrogenase 3 (NAD(+)) gamma(IDH3G) | −0.16866 |
| IDI1 | isopentenyl-diphosphate delta isomerase 1(IDI1) | 0.143824 |
| IDNK | IDNK, gluconokinase(IDNK) | −0.33646 |
| IDO1 | indoleamine 2,3-dioxygenase 1(IDO1) | −0.32191 |
| IDS | iduronate 2-sulfatase(IDS) | 0.071509 |
| IDUA | iduronidase, alpha-L-(IDUA) | −0.36579 |
| ILKAP | ILK associated serine/threonine phosphatase(ILKAP) | −0.30036 |
| ILVBL | ilvB acetolactate synthase like(ILVBL) | −0.33908 |
| IMPA1 | inositol monophosphatase 1(IMPA1) | −0.46081 |
| IMPA2 | inositol monophosphatase 2(IMPA2) | −0.12656 |
| IMPDH1 | inosine monophosphate dehydrogenase 1(IMPDH1) | −0.45932 |
| IMPDH2 | inosine monophosphate dehydrogenase 2(IMPDH2) | −0.29253 |
| INPP1 | inositol polyphosphate-1-phosphatase(INPP1) | −0.22436 |
| INPP4A | inositol polyphosphate-4-phosphatase type I A(INPP4A) | 0.020974 |
| INPP4B | inositol polyphosphate-4-phosphatase type II B(INPP4B) | −0.33864 |
| INPP5A | inositol polyphosphate-5-phosphatase A(INPP5A) | −0.24512 |
| INPP5B | inositol polyphosphate-5-phosphatase B(INPP5B) | 0.066304 |
| INPP5E | inositol polyphosphate-5-phosphatase E(INPP5E) | −0.47757 |
| INPP5F | inositol polyphosphate-5-phosphatase F(INPP5F) | −0.34789 |
| INPP5J | inositol polyphosphate-5-phosphatase J(INPP5J) | −0.00867 |
| INPP5K | inositol polyphosphate-5-phosphatase K(INPP5K) | −0.01779 |
| INPPL1 | inositol polyphosphate phosphatase like 1(INPPL1) | −0.35903 |
| ISYNA1 | inositol-3-phosphate synthase 1(ISYNA1) | −0.47013 |
| ITIH6 | inter-alpha-trypsin inhibitor heavy chain family member 6(ITIH6) | −0.08724 |
| ITPA | inosine triphosphatase(ITPA) | −0.18784 |
| ITPK1 | inositol-tetrakisphosphate 1-kinase(ITPK1) | −0.44228 |
| ITPKA | inositol-trisphosphate 3-kinase A(ITPKA) | −0.23444 |
| ITPKB | inositol-trisphosphate 3-kinase B(ITPKB) | −0.03765 |
| ITPKC | inositol-trisphosphate 3-kinase C(ITPKC) | −0.19671 |
| ITPR1 | inositol 1,4,5-trisphosphate receptor type 1(ITPR1) | −0.05171 |
| ITPR2 | inositol 1,4,5-trisphosphate receptor type 2(ITPR2) | −0.1657 |
| ITPR3 | inositol 1,4,5-trisphosphate receptor type 3(ITPR3) | −0.24994 |
| IVD | isovaleryl-CoA dehydrogenase(IVD) | −0.14575 |
| KCNA1 | potassium voltage-gated channel subfamily A member 1(KCNA1) | −0.27331 |
| KCNA10 | potassium voltage-gated channel subfamily A member 10(KCNA10) | 0.238246 |
| KCNA2 | potassium voltage-gated channel subfamily A member 2(KCNA2) | −0.24772 |
| KCNA3 | potassium voltage-gated channel subfamily A member 3(KCNA3) | −0.36193 |
| KCNA4 | potassium voltage-gated channel subfamily A member 4(KCNA4) | −0.34446 |
| KCNA5 | potassium voltage-gated channel subfamily A member 5(KCNA5) | 0.006843 |
| KCNA6 | potassium voltage-gated channel subfamily A member 6(KCNA6) | 0.018267 |
| KCNA7 | potassium voltage-gated channel subfamily A member 7(KCNA7) | −0.05952 |
| KCNAB1 | potassium voltage-gated channel subfamily A member regulatory beta subun [symbol] | −0.17679 |
| KCNAB2 | potassium voltage-gated channel subfamily A regulatory beta subunit 2(KCNA [symbol] | −0.41835 |
| KCNAB3 | potassium voltage-gated channel subfamily A regulatory beta subunit 3(KCNA [symbol] | −0.02061 |
| KCNB1 | potassium voltage-gated channel subfamily B member 1(KCNB1) | −0.17523 |
| KCNB2 | potassium voltage-gated channel subfamily B member 2(KCNB2) | −0.40948 |
| KCNC1 | potassium voltage-gated channel subfamily C member 1(KCNC1) | −0.23318 |
| KCNC2 | potassium voltage-gated channel subfamily C member 2(KCNC2) | −0.12095 |
| KCNC3 | potassium voltage-gated channel subfamily C member 3(KCNC3) | −0.27876 |
| KCNC4 | potassium voltage-gated channel subfamily C member 4(KCNC4) | 0.050421 |
| KCND1 | potassium voltage-gated channel subfamily D member 1(KCND1) | −0.25014 |
| KCND2 | potassium voltage-gated channel subfamily D member 2(KCND2) | −0.12724 |
| KCND3 | potassium voltage-gated channel subfamily D member 3(KCND3) | 0.136173 |
| KCNE1 | potassium voltage-gated channel subfamily E regulatory subunit 1(KCNE1) | −0.05056 |
| KCNE1L | | −0.16039 |
| KCNE2 | potassium voltage-gated channel subfamily E regulatory subunit 2(KCNE2) | −0.17071 |
| KCNE3 | potassium voltage-gated channel subfamily E regulatory subunit 3(KCNE3) | −0.29425 |
| KCNE4 | potassium voltage-gated channel subfamily E regulatory subunit 4(KCNE4) | −0.00375 |
| KCNF1 | potassium voltage-gated channel modifier subfamily F member 1(KCNF1) | −0.007 |
| KCNG1 | potassium voltage-gated channel modifier subfamily G member 1(KCNG1) | −0.0171 |
| KCNG2 | potassium voltage-gated channel modifier subfamily G member 2(KCNG2) | 0.017826 |
| KCNG3 | potassium voltage-gated channel modifier subfamily G member 3(KCNG3) | −0.16914 |
| KCNG4 | potassium voltage-gated channel modifier subfamily G member 4(KCNG4) | −0.45145 |
| KCNH1 | potassium voltage-gated channel subfamily H member 1(KCNH1) | −0.33771 |
| KCNH2 | potassium voltage-gated channel subfamily H member 2(KCNH2) | −0.09679 |
| KCNH3 | potassium voltage-gated channel subfamily H member 3(KCNH3) | 0.002693 |
| KCNH4 | potassium voltage-gated channel subfamily H member 4(KCNH4) | −0.1763 |
| KCNH5 | potassium voltage-gated channel subfamily H member 5(KCNH5) | −0.475 |
| KCNH6 | potassium voltage-gated channel subfamily H member 6(KCNH6) | −0.13441 |
| KCNH7 | potassium voltage-gated channel subfamily H member 7(KCNH7) | −0.63862 |
| KCNH8 | potassium voltage-gated channel subfamily H member 8(KCNH8) | 0.024066 |
| KCNJ1 | potassium voltage-gated channel subfamily J member 1(KCNJ1) | −0.03015 |
| KCNJ10 | potassium voltage-gated channel subfamily J member 10(KCNJ10) | −0.08698 |
| KCNJ11 | potassium voltage-gated channel subfamily J member 11(KCNJ11) | −0.31713 |
| KCNJ12 | potassium voltage-gated channel subfamily J member 12(KCNJ12) | −0.27649 |
| KCNJ13 | potassium voltage-gated channel subfamily J member 13(KCNJ13) | −0.38786 |
| KCNJ14 | potassium voltage-gated channel subfamily J member 14(KCNJ14) | 0.045668 |
| KCNJ15 | potassium voltage-gated channel subfamily J member 15(KCNJ15) | −0.3144 |
| KCNJ16 | potassium voltage-gated channel subfamily J member 16(KCNJ16) | 0.139898 |
| KCNJ2 | potassium voltage-gated channel subfamily J member 2(KCNJ2) | −0.35502 |
| KCNJ3 | potassium voltage-gated channel subfamily J member 3(KCNJ3) | 0.22537 |
| KCNJ4 | potassium voltage-gated channel subfamily J member 4(KCNJ4) | −0.20549 |
| KCNJ5 | potassium voltage-gated channel subfamily J member 5(KCNJ5) | −0.19165 |
| KCNJ6 | potassium voltage-gated channel subfamily J member 6(KCNJ6) | −0.13958 |
| KCNJ8 | potassium voltage-gated channel subfamily J member 8(KCNJ8) | −0.131 |
| KCNJ9 | potassium voltage-gated channel subfamily J member 9(KCNJ9) | −0.41871 |
| KCNK1 | potassium two pore domain channel subfamily K member 1(KCNK1) | −0.35877 |
| KCNK10 | potassium two pore domain channel subfamily K member 10(KCNK10) | −0.09171 |
| KCNK12 | potassium two pore domain channel subfamily K member 12(KCNK12) | −0.19472 |
| KCNK13 | potassium two pore domain channel subfamily K member 13(KCNK13) | −0.33011 |
| KCNK15 | potassium two pore domain channel subfamily K member 15(KCNK15) | −0.24717 |
| KCNK16 | potassium two pore domain channel subfamily K member 16(KCNK16) | 0.021937 |
| KCNK17 | potassium two pore domain channel subfamily K member 17(KCNK17) | −0.27539 |
| KCNK18 | potassium two pore domain channel subfamily K member 18(KCNK18) | −0.20103 |
| KCNK2 | potassium two pore domain channel subfamily K member 2(KCNK2) | −0.35945 |
| KCNK3 | potassium two pore domain channel subfamily K member 3(KCNK3) | −0.18371 |
| KCNK4 | potassium two pore domain channel subfamily K member 4(KCNK4) | 0.081089 |
| KCNK5 | potassium two pore domain channel subfamily K member 5(KCNK5) | −0.17794 |
| KCNK6 | potassium two pore domain channel subfamily K member 6(KCNK6) | −0.33902 |
| KCNK7 | potassium two pore domain channel subfamily K member 7(KCNK7) | −0.2685 |
| KCNK9 | potassium two pore domain channel subfamily K member 9(KCNK9) | −0.27647 |
| KCNMA1 | potassium calcium-activated channel subfamily M alpha 1(KCNMA1) | −0.20594 |
| KCNMB1 | potassium calcium-activated channel subfamily M regulatory beta subunit 1(k [symbol] | 0.0184 |
| KCNMB2 | potassium calcium-activated channel subfamily M regulatory beta subunit 2(k [symbol] | −0.00936 |
| KCNMB3 | potassium calcium-activated channel subfamily M regulatory beta subunit 3(k [symbol] | −0.48214 |
| KCNMB4 | potassium calcium-activated channel subfamily M regulatory beta subunit 4(k [symbol] | −0.30534 |
| KCNN2 | potassium calcium-activated channel subfamily N member 2(KCNN2) | −0.20837 |
| KCNN3 | potassium calcium-activated channel subfamily N member 3(KCNN3) | −0.15633 |
| KCNN4 | potassium calcium-activated channel subfamily N member 4(KCNN4) | −0.33808 |
| KCNQ1 | potassium voltage-gated channel subfamily Q member 1(KCNQ1) | −0.07841 |
| KCNQ2 | potassium voltage-gated channel subfamily Q member 2(KCNQ2) | −0.06308 |
| KCNQ3 | potassium voltage-gated channel subfamily Q member 3(KCNQ3) | −0.37439 |
| KCNQ4 | potassium voltage-gated channel subfamily Q member 4(KCNQ4) | −0.01267 |
| KCNQ5 | potassium voltage-gated channel subfamily Q member 5(KCNQ5) | −0.21663 |
| KCNS1 | potassium voltage-gated channel modifier subfamily S member 1(KCNS1) | −0.11277 |
| KCNS2 | potassium voltage-gated channel modifier subfamily S member 2(KCNS2) | −0.05155 |
| KCNS3 | potassium voltage-gated channel modifier subfamily S member 3(KCNS3) | −0.71274 |
| KCNT1 | potassium sodium-activated channel subfamily T member 1(KCNT1) | −0.02518 |
| KCNT2 | potassium sodium-activated channel subfamily T member 2(KCNT2) | −0.15184 |
| KCNU1 | potassium calcium-activated channel subfamily U member 1(KCNU1) | −0.17473 |
| KCNV1 | potassium voltage-gated channel modifier subfamily V member 1(KCNV1) | −0.22838 |
| KCNV2 | potassium voltage-gated channel modifier subfamily V member 2(KCNV2) | 0.089559 |
| KCTD5 | potassium channel tetramerization domain containing 5(KCTD5) | −0.19839 |
| KDM1A | lysine demethylase 1A(KDM1A) | −0.46114 |
| KDM1B | lysine demethylase 1B(KDM1B) | −0.22103 |
| KDSR | 3-ketodihydrosphingosine reductase(KDSR) | −0.17271 |
| KHK | ketohexokinase(KHK) | −0.09705 |
| KMO | kynurenine 3-monooxygenase(KMO) | −0.27825 |
| KRTAP17-1 | keratin associated protein 17-1(KRTAP17-1) | −0.09572 |
| KYNU | kynureninase(KYNU) | −0.39522 |
| L2HGDH | L-2-hydroxyglutarate dehydrogenase(L2HGDH) | −0.20509 |
| LALBA | lactalbumin alpha(LALBA) | −0.02394 |
| LARGE | | −0.10544 |
| LBR | lamin B receptor(LBR) | −0.42978 |
| LBX1 | ladybird homeobox 1(LBX1) | −0.31873 |
| LCAT | lecithin-cholesterol acyltransferase(LCAT) | −0.29708 |
| LCK | LCK proto-oncogene, Src family tyrosine kinase(LCK) | −0.01949 |
| LCT | lactase(LCT) | 0.00913 |
| LCTL | lactase like(LCTL) | −0.15874 |
| LDHA | lactate dehydrogenase A(LDHA) | −0.27285 |
| LDHB | lactate dehydrogenase B(LDHB) | −0.18696 |
| LDHC | lactate dehydrogenase C(LDHC) | 0.094979 |
| LGSN | lengsin, lens protein with glutamine synthetase domain(LGSN) | −0.18314 |
| LIPA | lipase A, lysosomal acid type(LIPA) | −0.63039 |
| LIPC | lipase C, hepatic type(LIPC) | −0.08537 |
| LIPE | lipase E, hormone sensitive type(LIPE) | −0.5632 |
| LIPF | lipase F, gastric type(LIPF) | −0.01813 |
| LIPG | lipase G, endothelial type(LIPG) | −0.20178 |
| LIPH | lipase H(LIPH) | −0.12384 |
| LIPI | lipase I(LIPI) | −0.36516 |
| LIPJ | lipase family member J(LIPJ) | −0.07425 |
| LIPK | lipase family member K(LIPK) | −0.07006 |
| LIPM | lipase family member M(LIPM) | −0.22602 |
| LIPN | lipase family member N(LIPN) | −0.06921 |
| LNPEP | leucyl and cystinyl aminopeptidase(LNPEP) | −0.24271 |
| LPCAT3 | lysophosphatidylcholine acyltransferase 3(LPCAT3) | 0.013567 |
| LPL | lipoprotein lipase(LPL) | −0.2554 |
| LPO | lactoperoxidase(LPO) | −0.09302 |
| LPPR2 | | 0.17383 |
| LRAT | lecithin retinol acyltransferase (phosphatidylcholine--retinol O-acyltransferas [symbol] | −0.21493 |
| LRPPRC | leucine rich pentatricopeptide repeat containing(LRPPRC) | −0.1296 |
| LSS | lanosterol synthase (2,3-oxidosqualene-lanosterol cyclase)(LSS) | −0.22387 |
| LTA4H | leukotriene A4 hydrolase(LTA4H) | −0.03936 |
| LTC4S | leukotriene C4 synthase(LTC4S) | −0.1779 |
| LYPLA1 | lysophospholipase I(LYPLA1) | −0.02435 |
| LYPLA2 | lysophospholipase II(LYPLA2) | −0.17948 |
| LYZ | lysozyme(LYZ) | −0.22609 |
| MAGEB4 | MAGE family member B4(MAGEB4) | −0.03781 |
| MAN1A2 | mannosidase alpha class 1A member 2(MAN1A2) | −0.25609 |
| MAN2A1 | mannosidase alpha class 2A member 1(MAN2A1) | −0.21585 |
| MAN2A2 | mannosidase alpha class 2A member 2(MAN2A2) | −0.1806 |
| MAN2B1 | mannosidase alpha class 2B member 1(MAN2B1) | −0.33689 |
| MAN2B2 | mannosidase alpha class 2B member 2(MAN2B2) | 0.0395 |
| MAN2C1 | mannosidase alpha class 2C member 1(MAN2C1) | −0.21282 |
| MANBA | mannosidase beta(MANBA) | −0.08253 |
| MAOA | monoamine oxidase A(MAOA) | −0.20866 |
| MAOB | monoamine oxidase B(MAOB) | −0.29928 |
| MAP2K1 | mitogen-activated protein kinase kinase 1(MAP2K1) | −0.22931 |
| MAT1A | methionine adenosyltransferase 1A(MAT1A) | −0.08136 |
| MAT2A | methionine adenosyltransferase 2A(MAT2A) | −0.00183 |
| MBOAT1 | membrane bound O-acyltransferase domain containing 1(MBOAT1) | −0.34756 |
| MBOAT2 | membrane bound O-acyltransferase domain containing 2(MBOAT2) | 0.020467 |
| MBOAT4 | membrane bound O-acyltransferase domain containing 4(MBOAT4) | −0.14909 |
| MBOAT7 | membrane bound O-acyltransferase domain containing 7(MBOAT7) | −0.14197 |
| MCAT | malonyl-CoA-acyl carrier protein transacylase(MCAT) | −0.04136 |
| MCCC1 | methylcrotonoyl-CoA carboxylase 1(MCCC1) | −0.0878 |
| MCCC2 | methylcrotonoyl-CoA carboxylase 2(MCCC2) | −0.1144 |
| MCEE | methylmalonyl-CoA epimerase(MCEE) | −0.42777 |
| MCOLN1 | mucolipin 1(MCOLN1) | −0.40225 |
| MCOLN2 | mucolipin 2(MCOLN2) | −0.34569 |
| MCOLN3 | mucolipin 3(MCOLN3) | −0.0397 |
| MDH1 | malate dehydrogenase 1(MDH1) | −0.12847 |
| MDH1B | malate dehydrogenase 1B(MDH1B) | −0.08824 |
| MDH2 | malate dehydrogenase 2(MDH2) | 0.032717 |
| MDP1 | magnesium dependent phosphatase 1(MDP1) | −0.2117 |
| ME1 | malic enzyme 1(ME1) | −0.23761 |
| ME2 | malic enzyme 2(ME2) | −0.76729 |
| ME3 | malic enzyme 3(ME3) | −0.07369 |
| MECR | mitochondrial trans-2-enoyl-CoA reductase(MECR) | −0.43901 |
| MFNG | MFNG O-fucosylpeptide 3-beta-N-acetylglucosaminyltransferase(MFNG) | −0.21052 |
| MFSD1 | major facilitator superfamily domain containing 1(MFSD1) | −0.14475 |
| MFSD10 | major facilitator superfamily domain containing 10(MFSD10) | −0.31372 |
| MFSD11 | major facilitator superfamily domain containing 11(MFSD11) | −0.4223 |
| MFSD3 | major facilitator superfamily domain containing 3(MFSD3) | −0.48824 |
| MFSD4 | major facilitator superfamily domain containing 4(MFSD4) | 0.20165 |
| MFSD5 | major facilitator superfamily domain containing 5(MFSD5) | −0.20423 |
| MFSD7 | major facilitator superfamily domain containing 7(MFSD7) | −0.29756 |
| MFSD8 | major facilitator superfamily domain containing 8(MFSD8) | 0.014902 |
| MFSD9 | major facilitator superfamily domain containing 9(MFSD9) | −0.02657 |
| MGAM | maltase-glucoamylase(MGAM) | −0.10213 |
| MGAT1 | mannosyl (alpha-1,3-)-glycoprotein beta-1,2-N-acetylglucosaminyltransferase | −0.11918 |
| MGAT4A | mannosyl (alpha-1,3-)-glycoprotein beta-1,4-N-acetylglucosaminyltransferase | −0.21881 |
| MGLL | monoglyceride lipase(MGLL) | −0.18671 |
| MGST1 | microsomal glutathione S-transferase 1(MGST1) | 0.042427 |
| MGST2 | microsomal glutathione S-transferase 2(MGST2) | −0.31268 |
| MGST3 | microsomal glutathione S-transferase 3(MGST3) | −0.29944 |
| MICAL2 | microtubule associated monooxygenase, calponin and LIM domain containing | −0.5127 |
| MICAL3 | microtubule associated monooxygenase, calponin and LIM domain containing | −0.36753 |
| MINPP1 | multiple inositol-polyphosphate phosphatase 1(MINPP1) | −0.31598 |
| MIP | major intrinsic protein of lens fiber(MIP) | −0.20446 |
| MLST8 | MTOR associated protein, LST8 homolog(MLST8) | −0.07264 |
| MLYCD | malonyl-CoA decarboxylase(MLYCD) | −0.01129 |
| MMAA | methylmalonic aciduria (cobalamin deficiency) cblA type(MMAA) | −0.1566 |
| MMAB | methylmalonic aciduria (cobalamin deficiency) cblB type(MMAB) | −0.28321 |
| MME | membrane metalloendopeptidase(MME) | −0.39113 |
| MMEL1 | membrane metalloendopeptidase like 1(MMEL1) | 0.099197 |
| MOCOS | molybdenum cofactor sulfurase(MOCOS) | −0.07703 |
| MOCS1 | molybdenum cofactor synthesis 1(MOCS1) | −0.24292 |
| MOCS2 | molybdenum cofactor synthesis 2(MOCS2) | 0.0667 |
| MOCS3 | molybdenum cofactor synthesis 3(MOCS3) | −0.5285 |
| MOGAT2 | monoacylglycerol O-acyltransferase 2(MOGAT2) | 0.066345 |
| MOGS | mannosyl-oligosaccharide glucosidase(MOGS) | −0.38532 |
| MPI | mannose phosphate isomerase(MPI) | −0.28481 |
| MPO | myeloperoxidase(MPO) | 0.027758 |
| MPST | mercaptopyruvate sulfurtransferase(MPST) | 0.149028 |
| MSMO1 | methylsterol monooxygenase 1(MSMO1) | −0.12732 |
| MSRA | methionine sulfoxide reductase A(MSRA) | −0.34266 |
| MTAP | methylthioadenosine phosphorylase(MTAP) | −0.16788 |
| MTHFD1 | methylenetetrahydrofolate dehydrogenase, cyclohydrolase and formyltetrah [symbol] | 0.021844 |
| MTHFD1L | methylenetetrahydrofolate dehydrogenase (NADP+ dependent) 1-like(MTHF [symbol] | −0.52991 |
| MTHFD2 | methylenetetrahydrofolate dehydrogenase (NADP+ dependent) 2, methenylt [symbol] | −0.27655 |
| MTHFD2L | methylenetetrahydrofolate dehydrogenase (NADP+ dependent) 2-like(MTHF [symbol] | −0.13374 |
| MTHFR | methylenetetrahydrofolate reductase(MTHFR) | −0.04978 |
| MTHFS | 5,10-methenyltetrahydrofolate synthetase (5-formyltetrahydrofolate cyclo-lig [symbol] | −0.43164 |
| MTHFSD | methenyltetrahydrofolate synthetase domain containing(MTHFSD) | 0.004663 |
| MTM1 | myotubularin 1(MTM1) | −0.29897 |
| MTMR1 | myotubularin related protein 1(MTMR1) | −0.44154 |
| MTMR14 | myotubularin related protein 14(MTMR14) | −0.26356 |
| MTMR2 | myotubularin related protein 2(MTMR2) | 0.070958 |
| MTMR3 | myotubularin related protein 3(MTMR3) | −0.13915 |
| MTMR4 | myotubularin related protein 4(MTMR4) | 0.160298 |
| MTMR6 | myotubularin related protein 6(MTMR6) | −0.79513 |
| MTMR7 | myotubularin related protein 7(MTMR7) | −0.22616 |
| MTMR8 | myotubularin related protein 8(MTMR8) | −0.31382 |
| MTR | 5-methyltetrahydrofolate-homocysteine methyltransferase(MTR) | −0.06938 |
| MTRR | 5-methyltetrahydrofolate-homocysteine methyltransferase reductase(MTRR) | 0.226309 |
| MTTP | microsomal triglyceride transfer protein(MTTP) | −0.21256 |
| MUT | methylmalonyl-CoA mutase(MUT) | −0.27908 |
| MVK | mevalonate kinase(MVK) | −0.27805 |
| NAAA | N-acylethanolamine acid amidase(NAAA) | −0.03904 |
| NAALAD2 | N-acetylated alpha-linked acidic dipeptidase 2(NAALAD2) | −0.19851 |
| NAALADL1 | N-acetylated alpha-linked acidic dipeptidase like 1(NAALADL1) | −0.40588 |
| NAALADL2 | N-acetylated alpha-linked acidic dipeptidase like 2(NAALADL2) | −0.10903 |
| NADK | NAD kinase(NADK) | −0.35293 |
| NAGA | alpha-N-acetylgalactosaminidase(NAGA) | −0.08581 |
| NAGK | N-acetylglucosamine kinase(NAGK) | −0.49223 |
| NAGLU | N-acetyl-alpha-glucosaminidase(NAGLU) | −0.62091 |
| NAGS | N-acetylglutamate synthase(NAGS) | −0.08259 |
| NALCN | sodium leak channel, non-selective(NALCN) | −0.33966 |
| NANP | N-acetylneuraminic acid phosphatase(NANP) | 0.22161 |
| NANS | N-acetylneuraminate synthase(NANS) | −0.5515 |
| NDOR1 | NADPH dependent diflavin oxidoreductase 1(NDOR1) | −0.22782 |
| NDST4 | N-deacetylase and N-sulfotransferase 4(NDST4) | −0.2843 |
| NDUFA1 | NADH:ubiquinone oxidoreductase subunit A1(NDUFA1) | −0.31908 |
| NDUFA10 | NADH:ubiquinone oxidoreductase subunit A10(NDUFA10) | −0.44885 |
| NDUFA11 | NADH:ubiquinone oxidoreductase subunit A11(NDUFA11) | −1.18712 |
| NDUFA12 | NADH:ubiquinone oxidoreductase subunit A12(NDUFA12) | −0.55243 |
| NDUFA13 | NADH:ubiquinone oxidoreductase subunit A13(NDUFA13) | −0.12071 |
| NDUFA2 | NADH:ubiquinone oxidoreductase subunit A2(NDUFA2) | −0.01511 |
| NDUFA3 | NADH:ubiquinone oxidoreductase subunit A3(NDUFA3) | −0.17476 |
| NDUFA4 | NDUFA4, mitochondrial complex associated(NDUFA4) | −0.09221 |
| NDUFA4L2 | NDUFA4, mitochondrial complex associated like 2(NDUFA4L2) | −0.10962 |
| NDUFA5 | NADH:ubiquinone oxidoreductase subunit A5(NDUFA5) | −0.11613 |
| NDUFA6 | NADH:ubiquinone oxidoreductase subunit A6(NDUFA6) | −0.05721 |
| NDUFA7 | NADH:ubiquinone oxidoreductase subunit A7(NDUFA7) | −0.22437 |
| NDUFA8 | NADH:ubiquinone oxidoreductase subunit A8(NDUFA8) | −0.05686 |
| NDUFA9 | NADH:ubiquinone oxidoreductase subunit A9(NDUFA9) | −0.07559 |
| NDUFAB1 | NADH:ubiquinone oxidoreductase subunit AB1(NDUFAB1) | −0.42718 |
| NDUFAF1 | NADH:ubiquinone oxidoreductase complex assembly factor 1(NDUFAF1) | 0.231179 |
| NDUFAF2 | NADH:ubiquinone oxidoreductase complex assembly factor 2(NDUFAF2) | −0.23987 |
| NDUFAF3 | NADH:ubiquinone oxidoreductase complex assembly factor 3(NDUFAF3) | −0.29843 |
| NDUFAF4 | NADH:ubiquinone oxidoreductase complex assembly factor 4(NDUFAF4) | 0.115808 |
| NDUFB1 | NADH:ubiquinone oxidoreductase subunit B1(NDUFB1) | −0.45999 |
| NDUFB10 | NADH:ubiquinone oxidoreductase subunit B10(NDUFB10) | −0.50518 |
| NDUFB11 | NADH:ubiquinone oxidoreductase subunit B11(NDUFB11) | −0.09282 |
| NDUFB2 | NADH:ubiquinone oxidoreductase subunit B2(NDUFB2) | −0.13339 |
| NDUFB3 | NADH:ubiquinone oxidoreductase subunit B3(NDUFB3) | −0.45924 |
| NDUFB4 | NADH:ubiquinone oxidoreductase subunit B4(NDUFB4) | −0.03256 |
| NDUFB5 | NADH:ubiquinone oxidoreductase subunit B5(NDUFB5) | −0.32613 |
| NDUFB6 | NADH:ubiquinone oxidoreductase subunit B6(NDUFB6) | −0.0019 |
| NDUFB7 | NADH:ubiquinone oxidoreductase subunit B7(NDUFB7) | 0.03951 |
| NDUFB8 | NADH:ubiquinone oxidoreductase subunit B8(NDUFB8) | −0.45727 |
| NDUFB9 | NADH:ubiquinone oxidoreductase subunit B9(NDUFB9) | −0.34087 |
| NDUFC1 | NADH:ubiquinone oxidoreductase subunit C1(NDUFC1) | −0.50526 |
| NDUFC2 | NADH:ubiquinone oxidoreductase subunit C2(NDUFC2) | −0.07327 |
| NDUFS1 | NADH:ubiquinone oxidoreductase core subunit S1(NDUFS1) | −0.29544 |
| NDUFS2 | NADH:ubiquinone oxidoreductase core subunit S2(NDUFS2) | −0.32548 |
| NDUFS3 | NADH:ubiquinone oxidoreductase core subunit S3(NDUFS3) | −0.16577 |
| NDUFS4 | NADH:ubiquinone oxidoreductase subunit S4(NDUFS4) | −0.04026 |
| NDUFS5 | NADH:ubiquinone oxidoreductase subunit S5(NDUFS5) | −0.13193 |
| NDUFS6 | NADH:ubiquinone oxidoreductase subunit S6(NDUFS6) | −0.461 |
| NDUFS7 | NADH:ubiquinone oxidoreductase core subunit S7(NDUFS7) | −0.31836 |
| NDUFS8 | NADH:ubiquinone oxidoreductase core subunit S8(NDUFS8) | −0.46876 |
| NDUFV1 | NADH:ubiquinone oxidoreductase core subunit V1(NDUFV1) | −0.25199 |
| NDUFV2 | NADH:ubiquinone oxidoreductase core subunit V2(NDUFV2) | 0.31911 |
| NDUFV3 | NADH:ubiquinone oxidoreductase subunit V3(NDUFV3) | 0.220559 |
| NEU1 | neuraminidase 1(NEU1) | −0.16284 |
| NIPA1 | non imprinted in Prader-Willi/Angelman syndrome 1(NIPA1) | −0.15074 |
| NIT2 | nitrilase family member 2(NIT2) | −0.27132 |
| NKX3-1 | NK3 homeobox 1(NKX3-1) | −0.40509 |
| NME1 | NME/NM23 nucleoside diphosphate kinase 1(NME1) | 0.147715 |
| NME2 | NME/NM23 nucleoside diphosphate kinase 2(NME2) | 0.119101 |
| NME3 | NME/NM23 nucleoside diphosphate kinase 3(NME3) | −0.00738 |
| NME4 | NME/NM23 nucleoside diphosphate kinase 4(NME4) | −0.04773 |
| NME5 | NME/NM23 family member 5(NME5) | −0.09527 |
| NME6 | NME/NM23 nucleoside diphosphate kinase 6(NME6) | 0.41342 |
| NME7 | NME/NM23 family member 7(NME7) | −0.39933 |
| NNMT | nicotinamide N-methyltransferase(NNMT) | −0.06364 |
| NNT | nicotinamide nucleotide transhydrogenase(NNT) | −0.08537 |
| NOS1 | nitric oxide synthase 1(NOS1) | 0.07825 |
| NOS2 | nitric oxide synthase 2(NOS2) | −0.04513 |
| NOS3 | nitric oxide synthase 3(NOS3) | −0.04787 |
| NOX1 | NADPH oxidase 1(NOX1) | −0.22043 |
| NOX4 | NADPH oxidase 4(NOX4) | −0.44935 |
| NOX5 | NADPH oxidase 5(NOX5) | −0.43324 |
| NPC1 | NPC intracellular cholesterol transporter 1(NPC1) | −1.84352 |
| NPC1L1 | NPC1 like intracellular cholesterol transporter 1(NPC1L1) | −0.03469 |
| NPC2 | NPC intracellular cholesterol transporter 2(NPC2) | −0.65316 |
| NPL | N-acetylneuraminate pyruvate lyase(NPL) | −0.22593 |
| NPR1 | natriuretic peptide receptor 1(NPR1) | −0.17045 |
| NPR2 | natriuretic peptide receptor 2(NPR2) | −0.21199 |
| NPR3 | natriuretic peptide receptor 3(NPR3) | −0.10477 |
| NPVF | neuropeptide VF precursor(NPVF) | −0.27795 |
| NQ01 | NAD(P)H quinone dehydrogenase 1(NQO1) | −0.48446 |
| NQO2 | NAD(P)H quinone dehydrogenase 2(NQO2) | −0.38188 |
| NROB1 | nuclear receptor subfamily 0 group B member 1(NROB1) | −0.24974 |
| NROB2 | nuclear receptor subfamily 0 group B member 2(NROB2) | −0.07267 |
| NR1D1 | nuclear receptor subfamily 1 group D member 1(NR1D1) | −0.18336 |
| NR1D2 | nuclear receptor subfamily 1 group D member 2(NR1D2) | −0.34304 |
| NR1H2 | nuclear receptor subfamily 1 group H member 2(NR1H2) | −0.14331 |
| NR1H3 | nuclear receptor subfamily 1 group H member 3(NR1H3) | −0.13063 |
| NR1H4 | nuclear receptor subfamily 1 group H member 4(NR1H4) | 0.06441 |
| NR112 | nuclear receptor subfamily 1 group I member 2(NR112) | −0.26827 |
| NR113 | nuclear receptor subfamily 1 group I member 3(NR113) | −0.25384 |
| NR2C1 | nuclear receptor subfamily 2 group C member 1(NR2C1) | 0.15543 |
| NR2C2 | nuclear receptor subfamily 2 group C member 2(NR2C2) | −0.19199 |
| NR2E1 | nuclear receptor subfamily 2 group E member 1(NR2E1) | −0.44256 |
| NR2F1 | nuclear receptor subfamily 2 group F member 1(NR2F1) | −0.12891 |
| NR2F2 | nuclear receptor subfamily 2 group F member 2(NR2F2) | −0.02533 |
| NR2F6 | nuclear receptor subfamily 2 group F member 6(NR2F6) | −0.42072 |
| NR3C1 | nuclear receptor subfamily 3 group C member 1(NR3C1) | −0.22123 |
| NR3C2 | nuclear receptor subfamily 3 group C member 2(NR3C2) | 0.282833 |
| NR4A1 | nuclear receptor subfamily 4 group A member 1(NR4A1) | 0.102271 |
| NR4A2 | nuclear receptor subfamily 4 group A member 2(NR4A2) | −0.16429 |
| NR4A3 | nuclear receptor subfamily 4 group A member 3(NR4A3) | −0.76038 |
| NR5A1 | nuclear receptor subfamily 5 group A member 1(NR5A1) | −0.9767 |
| NR5A2 | nuclear receptor subfamily 5 group A member 2(NR5A2) | −0.07546 |
| NR6A1 | nuclear receptor subfamily 6 group A member 1(NR6A1) | −0.37126 |
| NSDHL | NAD(P) dependent steroid dehydrogenase-like(NSDHL) | −0.47719 |
| NT5C1B | 5′-nucleotidase, cytosolic IB(NT5C1B) | −0.01902 |
| NT5C3A | 5′-nucleotidase, cytosolic IIIA(NT5C3A) | −0.27651 |
| NT5C3B | 5′-nucleotidase, cytosolic IIIB(NT5C3B) | −0.28712 |
| NT5DC2 | 5′-nucleotidase domain containing 2(NT5DC2) | −0.07156 |
| NT5E | 5′-nucleotidase ecto(NT5E) | −0.18993 |
| NUDT1 | nudix hydrolase 1(NUDT1) | −0.14201 |
| NUDT2 | nudix hydrolase 2(NUDT2) | 0.035867 |
| NUDT21 | nudix hydrolase 21(NUDT21) | 0.287426 |
| NUDT4 | nudix hydrolase 4(NUDT4) | −0.42777 |
| NUDT5 | nudix hydrolase 5(NUDT5) | 0.051121 |
| NUDT9 | nudix hydrolase 9(NUDT9) | 0.24649 |
| OAS1 | 2′-5′-oligoadenylate synthetase 1(OAS1) | −0.14107 |
| OAS2 | 2′-5′-oligoadenylate synthetase 2(OAS2) | −0.08858 |
| OAS3 | 2′-5′-oligoadenylate synthetase 3(OAS3) | 0.24967 |
| OASL | 2′-5′-oligoadenylate synthetase like(OASL) | −0.20293 |
| OAT | ornithine aminotransferase(OAT) | −0.314 |
| OCA2 | OCA2 melanosomal transmembrane protein(OCA2) | −0.11066 |
| ODC1 | ornithine decarboxylase 1(ODC1) | −0.14669 |
| OGDH | oxoglutarate dehydrogenase(OGDH) | −0.1561 |
| OGDHL | oxoglutarate dehydrogenase-like(OGDHL) | −0.31465 |
| OLAH | oleoyl-ACP hydrolase(OLAH) | −0.28243 |
| OTC | ornithine carbamoyltransferase(OTC) | −0.36813 |
| OXCT1 | 3-oxoacid CoA-transferase 1(OXCT1) | −0.35139 |
| OXCT2 | 3-oxoacid CoA-transferase 2(OXCT2) | 0.006216 |
| OXSM | 3-oxoacyl-ACP synthase, mitochondrial(OXSM) | −0.36199 |
| P2RX1 | purinergic receptor P2X 1(P2RX1) | −0.06289 |
| P2RX2 | purinergic receptor P2X 2(P2RX2) | −0.2218 |
| P2RX3 | purinergic receptor P2X 3(P2RX3) | −0.28357 |
| P2RX4 | purinergic receptor P2X 4(P2RX4) | 0.022662 |
| P2RX5 | purinergic receptor P2X 5(P2RX5) | −0.1321 |
| P2RX6 | purinergic receptor P2X 6(P2RX6) | −0.16477 |
| P2RX7 | purinergic receptor P2X 7(P2RX7) | −0.4059 |
| PAH | phenylalanine hydroxylase(PAH) | −0.22867 |
| PAICS | phosphoribosylaminoimidazole carboxylase; phosphoribosylaminoimidazoles | −0.8788 |
| PAM | peptidylglycine alpha-amidating monooxygenase(PAM) | −0.15357 |
| PANK1 | pantothenate kinase 1(PANK1) | −0.32298 |
| PANK2 | pantothenate kinase 2(PANK2) | −0.08841 |
| PANK3 | pantothenate kinase 3(PANK3) | −0.18762 |
| PANK4 | pantothenate kinase 4(PANK4) | −0.29344 |
| PAOX | polyamine oxidase(PAOX) | 0.16783 |
| PAPSS1 | 3′-phosphoadenosine 5′-phosphosulfate synthase 1(PAPSS1) | −0.39919 |
| PAPSS2 | 3′-phosphoadenosine 5′-phosphosulfate synthase 2(PAPSS2) | −0.33111 |
| PASD1 | PAS domain containing 1(PASD1) | 0.127314 |
| PC | pyruvate carboxylase(PC) | −0.18917 |
| PCBD1 | pterin-4 alpha-carbinolamine dehydratase 1(PCBD1) | −0.30156 |
| PCBD2 | pterin-4 alpha-carbinolamine dehydratase 2(PCBD2) | −0.12582 |
| PCCA | propionyl-CoA carboxylase alpha subunit(PCCA) | −0.49638 |
| PCCB | propionyl-CoA carboxylase beta subunit(PCCB) | −0.55635 |
| PCK1 | phosphoenolpyruvate carboxykinase 1(PCK1) | −0.1249 |
| PCK2 | phosphoenolpyruvate carboxykinase 2, mitochondrial(PCK2) | 0.097452 |
| PDE10A | phosphodiesterase 10A(PDE10A) | −0.17773 |
| PDE11A | phosphodiesterase 11A(PDE11A) | −0.15448 |
| PDE12 | phosphodiesterase 12(PDE12) | −0.25613 |
| PDE1A | phosphodiesterase 1A(PDE1A) | −0.49681 |
| PDE1B | phosphodiesterase 1B(PDE1B) | −0.61497 |
| PDE1C | phosphodiesterase 1C(PDE1C) | −0.19748 |
| PDE2A | phosphodiesterase 2A(PDE2A) | −0.25778 |
| PDE3A | phosphodiesterase 3A(PDE3A) | −0.10586 |
| PDE3B | phosphodiesterase 3B(PDE3B) | −0.12763 |
| PDE4A | phosphodiesterase 4A(PDE4A) | −0.1285 |
| PDE4B | phosphodiesterase 4B(PDE4B) | −0.3259 |
| PDE4C | phosphodiesterase 4C(PDE4C) | −0.11826 |
| PDE4D | phosphodiesterase 4D(PDE4D) | −0.36885 |
| PDE5A | phosphodiesterase 5A(PDE5A) | −0.38753 |
| PDE6A | phosphodiesterase 6A(PDE6A) | −0.11877 |
| PDE6B | phosphodiesterase 6B(PDE6B) | −0.06273 |
| PDE6C | phosphodiesterase 6C(PDE6C) | −0.24732 |
| PDE6D | phosphodiesterase 6D(PDE6D) | 0.023268 |
| PDE6G | phosphodiesterase 6G(PDE6G) | −0.06593 |
| PDE6H | phosphodiesterase 6H(PDE6H) | 0.052544 |
| PDE7A | phosphodiesterase 7A(PDE7A) | 0.101929 |
| PDE7B | phosphodiesterase 7B(PDE7B) | −0.24352 |
| PDE8A | phosphodiesterase 8A(PDE8A) | −0.11641 |
| PDE8B | phosphodiesterase 8B(PDE8B) | −0.15443 |
| PDE9A | phosphodiesterase 9A(PDE9A) | −0.21574 |
| PDHA1 | pyruvate dehydrogenase (lipoamide) alpha 1(PDHA1) | 0.30457 |
| PDHA2 | pyruvate dehydrogenase (lipoamide) alpha 2(PDHA2) | 0.03258 |
| PDHB | pyruvate dehydrogenase (lipoamide) beta(PDHB) | 0.150919 |
| PDHX | pyruvate dehydrogenase complex component X(PDHX) | −0.38853 |
| PDK1 | pyruvate dehydrogenase kinase 1(PDK1) | 0.171251 |
| PDK2 | pyruvate dehydrogenase kinase 2(PDK2) | −0.23707 |
| PDK3 | pyruvate dehydrogenase kinase 3(PDK3) | −0.32433 |
| PDK4 | pyruvate dehydrogenase kinase 4(PDK4) | 0.163996 |
| PDP1 | pyruvate dehyrogenase phosphatase catalytic subunit 1(PDP1) | −0.08036 |
| PDP2 | pyruvate dehyrogenase phosphatase catalytic subunit 2(PDP2) | −0.27542 |
| PDXK | pyridoxal (pyridoxine, vitamin B6) kinase(PDXK) | −0.22199 |
| PDXP | pyridoxal phosphatase(PDXP) | −0.09089 |
| PECR | peroxisomal trans-2-enoyl-CoA reductase(PECR) | −0.0675 |
| PFAS | phosphoribosylformylglycinamidine synthase(PFAS) | −0.24889 |
| PFKFB1 | 6-phosphofructo-2-kinase/fructose-2,6-biphosphatase 1(PFKFB1) | −0.13486 |
| PFKFB2 | 6-phosphofructo-2-kinase/fructose-2,6-biphosphatase 2(PFKFB2) | −0.20086 |
| PFKFB3 | 6-phosphofructo-2-kinase/fructose-2,6-biphosphatase 3(PFKFB3) | −0.20916 |
| PFKFB4 | 6-phosphofructo-2-kinase/fructose-2,6-biphosphatase 4(PFKFB4) | −0.15762 |
| PFKL | phosphofructokinase, liver type(PFKL) | −0.28188 |
| PFKM | phosphofructokinase, muscle(PFKM) | −0.39047 |
| PFKP | phosphofructokinase, platelet(PFKP) | −0.14481 |
| PGAM1 | phosphoglycerate mutase 1(PGAM1) | −0.13082 |
| PGAM2 | phosphoglycerate mutase 2(PGAM2) | 0.02172 |
| PGAM4 | phosphoglycerate mutase family member 4(PGAM4) | −0.04695 |
| PGAM5 | PGAM family member 5, mitochondrial serine/threonine protein phosphatase | −0.37337 |
| PGAP1 | post-GPI attachment to proteins 1(PGAP1) | −0.18265 |
| PGD | phosphogluconate dehydrogenase(PGD) | −0.8284 |
| PGK1 | phosphoglycerate kinase 1(PGK1) | 0.21179 |
| PGK2 | phosphoglycerate kinase 2(PGK2) | −0.00059 |
| PGLS | 6-phosphogluconolactonase(PGLS) | −0.15122 |
| PGM1 | phosphoglucomutase 1(PGM1) | −0.29049 |
| PGM2 | phosphoglucomutase 2(PGM2) | −0.16181 |
| PGM2L1 | phosphoglucomutase 2 like 1(PGM2L1) | −0.1576 |
| PGM3 | phosphoglucomutase 3(PGM3) | −0.51846 |
| PGM5 | phosphoglucomutase 5(PGM5) | −0.69266 |
| PGR | progesterone receptor(PGR) | 0.22924 |
| PHGDH | phosphoglycerate dehydrogenase(PHGDH) | −0.28173 |
| PHLPP1 | PH domain and leucine rich repeat protein phosphatase 1(PHLPP1) | −0.38626 |
| PHLPP2 | PH domain and leucine rich repeat protein phosphatase 2(PHLPP2) | −0.1672 |
| PHOSPHO1 | phosphoethanolamine/phosphocholine phosphatase(PHOSPHO1) | −0.31091 |
| PHPT1 | phosphohistidine phosphatase 1(PHPT1) | −0.15854 |
| PHYH | phytanoyl-CoA 2-hydroxylase(PHYH) | −0.21135 |
| PHYHD1 | phytanoyl-CoA dioxygenase domain containing 1(PHYHD1) | −0.21244 |
| PI4K2A | phosphatidylinositol 4-kinase type 2 alpha(PI4K2A) | −0.32169 |
| PI4K2B | phosphatidylinositol 4-kinase type 2 beta(PI4K2B) | 0.524306 |
| PI4KA | phosphatidylinositol 4-kinase alpha(PI4KA) | −0.41938 |
| PI4KB | phosphatidylinositol 4-kinase beta(PI4KB) | −1.1646 |
| PIGA | phosphatidylinositol glycan anchor biosynthesis class A(PIGA) | 0.113173 |
| PIGK | phosphatidylinositol glycan anchor biosynthesis class K(PIGK) | 0.053496 |
| PIK3C2A | phosphatidylinositol-4-phosphate 3-kinase catalytic subunit type 2 alpha(PIK3 [symbol] | 0.159876 |
| PIK3C2B | phosphatidylinositol-4-phosphate 3-kinase catalytic subunit type 2 beta(PIK3 [symbol] | −0.50764 |
| PIK3C2G | phosphatidylinositol-4-phosphate 3-kinase catalytic subunit type 2 gamma(PII [symbol] | −0.09632 |
| PIK3C3 | phosphatidylinositol 3-kinase catalytic subunit type 3(PIK3C3) | −0.47443 |
| PIK3CA | phosphatidylinositol-4,5-bisphosphate 3-kinase catalytic subunit alpha(PIK3C [symbol] | −0.35025 |
| PIK3CB | phosphatidylinositol-4,5-bisphosphate 3-kinase catalytic subunit beta(PIK3CB [symbol] | −0.08687 |
| PIK3CD | phosphatidylinositol-4,5-bisphosphate 3-kinase catalytic subunit delta(PIK3C [symbol] | 0.011877 |
| PIK3CG | phosphatidylinositol-4,5-bisphosphate 3-kinase catalytic subunit gamma(PIK3 [symbol] | 0.057871 |
| PIK3R1 | phosphoinositide-3-kinase regulatory subunit 1(PIK3R1) | −0.18743 |
| PIK3R2 | phosphoinositide-3-kinase regulatory subunit 2(PIK3R2) | −0.05148 |
| PIK3R3 | phosphoinositide-3-kinase regulatory subunit 3(PIK3R3) | −0.1558 |
| PIK3R4 | phosphoinositide-3-kinase regulatory subunit 4(PIK3R4) | −0.38532 |
| PIK3R5 | phosphoinositide-3-kinase regulatory subunit 5(PIK3R5) | −0.26312 |
| PIKFYVE | phosphoinositide kinase, FYVE-type zinc finger containing(PIKFYVE) | −0.32996 |
| PIP5K1A | phosphatidylinositol-4-phosphate 5-kinase type 1 alpha(PIP5K1A) | −0.56091 |
| PIP5K1B | phosphatidylinositol-4-phosphate 5-kinase type 1 beta(PIP5K1B) | −0.45752 |
| PIP5K1C | phosphatidylinositol-4-phosphate 5-kinase type 1 gamma(PIP5K1C) | 0.068314 |
| PIP5KL1 | phosphatidylinositol-4-phosphate 5-kinase like 1(PIP5KL1) | −0.01694 |
| PIPOX | pipecolic acid and sarcosine oxidase(PIPOX) | 0.01869 |
| PIWIL1 | piwi like RNA-mediated gene silencing 1(PIWIL1) | −0.26885 |
| PKD1 | polycystin 1, transient receptor potential channel interacting(PKD1) | 0.044533 |
| PKD2 | polycystin 2, transient receptor potential cation channel(PKD2) | −0.58013 |
| PKD2L1 | polycystin 2 like 1, transient receptor potential cation channel(PKD2L1) | −0.09033 |
| PKLR | pyruvate kinase, liver and RBC(PKLR) | −0.12263 |
| PKM | pyruvate kinase, muscle(PKM) | −0.08048 |
| PLA1A | phospholipase A1 member A(PLA1A) | 0.327006 |
| PLA2G10 | phospholipase A2 group X(PLA2G10) | −0.1797 |
| PLA2G12A | phospholipase A2 group XIIA(PLA2G12A) | −0.41791 |
| PLA2G1B | phospholipase A2 group IB(PLA2G1B) | −0.44834 |
| PLA2G2A | phospholipase A2 group IIA(PLA2G2A) | −0.28565 |
| PLA2G2D | phospholipase A2 group IID(PLA2G2D) | −0.1297 |
| PLA2G2E | phospholipase A2 group IIE(PLA2G2E) | −0.15687 |
| PLA2G2F | phospholipase A2 group IIF(PLA2G2F) | −0.09056 |
| PLA2G3 | phospholipase A2 group III(PLA2G3) | −0.28273 |
| PLA2G4A | phospholipase A2 group IVA(PLA2G4A) | −0.2829 |
| PLA2G4B | phospholipase A2 group IVB(PLA2G4B) | −0.02825 |
| PLA2G4C | phospholipase A2 group IVC(PLA2G4C) | −0.33694 |
| PLA2G5 | phospholipase A2 group V(PLA2G5) | 0.16282 |
| PLA2G6 | phospholipase A2 group VI(PLA2G6) | −0.25468 |
| PLA2G7 | phospholipase A2 group VII(PLA2G7) | −0.25151 |
| PLCB1 | phospholipase C beta 1(PLCB1) | −0.21716 |
| PLCB2 | phospholipase C beta 2(PLCB2) | −0.37965 |
| PLCB3 | phospholipase C beta 3(PLCB3) | 0.071219 |
| PLCB4 | phospholipase C beta 4(PLCB4) | −0.22636 |
| PLCD1 | phospholipase C delta 1(PLCD1) | −0.18287 |
| PLCD4 | phospholipase C delta 4(PLCD4) | −0.09908 |
| PLCE1 | phospholipase C epsilon 1(PLCE1) | −0.43205 |
| PLCG1 | phospholipase C gamma 1(PLCG1) | −0.00513 |
| PLCG2 | phospholipase C gamma 2(PLCG2) | −0.25279 |
| PLCH1 | phospholipase C eta 1(PLCH1) | −0.26804 |
| PLCL1 | phospholipase C like 1(PLCL1) | −0.22078 |
| PLCL2 | phospholipase C like 2(PLCL2) | −0.19185 |
| PLCZ1 | phospholipase C zeta 1(PLCZ1) | 0.041847 |
| PLD1 | phospholipase D1(PLD1) | −0.20152 |
| PLD2 | phospholipase D2(PLD2) | −0.00332 |
| PMM1 | phosphomannomutase 1(PMM1) | −0.34274 |
| PMM2 | phosphomannomutase 2(PMM2) | −0.28789 |
| PMVK | phosphomevalonate kinase(PMVK) | −0.49849 |
| PNLIP | pancreatic lipase(PNLIP) | −0.04945 |
| PNLIPRP1 | pancreatic lipase related protein 1(PNLIPRP1) | −0.15319 |
| PNLIPRP3 | pancreatic lipase related protein 3(PNLIPRP3) | −0.56103 |
| PNMT | phenylethanolamine N-methyltransferase(PNMT) | −0.17152 |
| PNPLA1 | patatin like phospholipase domain containing 1(PNPLA1) | −0.26439 |
| PNPLA2 | patatin like phospholipase domain containing 2(PNPLA2) | −0.25841 |
| PNPLA3 | patatin like phospholipase domain containing 3(PNPLA3) | −0.00311 |
| PNPLA4 | patatin like phospholipase domain containing 4(PNPLA4) | −0.07025 |
| PNPLA5 | patatin like phospholipase domain containing 5(PNPLA5) | −0.30367 |
| PNPLA6 | patatin like phospholipase domain containing 6(PNPLA6) | −1.50963 |
| PNPLA7 | patatin like phospholipase domain containing 7(PNPLA7) | −0.20196 |
| PNPLA8 | patatin like phospholipase domain containing 8(PNPLA8) | −0.52227 |
| POFUT1 | protein O-fucosyltransferase 1(POFUT1) | −0.20848 |
| POMT1 | protein O-mannosyltransferase 1(POMT1) | −0.22715 |
| PON1 | paraoxonase 1(PON1) | −0.20849 |
| PON2 | paraoxonase 2(PON2) | 0.082103 |
| PON3 | paraoxonase 3(PON3) | −0.2155 |
| POR | cytochrome p450 oxidoreductase(POR) | −0.31126 |
| PPA1 | pyrophosphatase (inorganic) 1(PPA1) | −0.0529 |
| PPA2 | pyrophosphatase (inorganic) 2(PPA2) | −0.34396 |
| PPAP2A | | 0.013903 |
| PPAP2B | | −0.00144 |
| PPAP2C | | 0.033078 |
| PPARA | peroxisome proliferator activated receptor alpha(PPARA) | 0.112629 |
| PPARD | peroxisome proliferator activated receptor delta(PPARD) | −0.18816 |
| PPARG | peroxisome proliferator activated receptor gamma(PPARG) | −0.19854 |
| PPAT | phosphoribosyl pyrophosphate amidotransferase(PPAT) | −0.34909 |
| PPCDC | phosphopantothenoylcysteine decarboxylase(PPCDC) | −0.50597 |
| PPCS | phosphopantothenoylcysteine synthetase(PPCS) | −0.35361 |
| PPEF1 | protein phosphatase with EF-hand domain 1(PPEF1) | −0.16315 |
| PPEF2 | protein phosphatase with EF-hand domain 2(PPEF2) | −0.08224 |
| PPM1A | protein phosphatase, Mg2+/Mn2+ dependent 1A(PPM1A) | −0.24301 |
| PPM1B | protein phosphatase, Mg2+/Mn2+ dependent 1B(PPM1B) | −0.13205 |
| PPM1D | protein phosphatase, Mg2+/Mn2+ dependent 1D(PPM1D) | −0.44979 |
| PPM1E | protein phosphatase, Mg2+/Mn2+ dependent 1E(PPM1E) | −0.13798 |
| PPM1F | protein phosphatase, Mg2+/Mn2+ dependent 1F(PPM1F) | −0.21809 |
| PPM1G | protein phosphatase, Mg2+/Mn2+ dependent 1G(PPM1G) | 0.215257 |
| PPM1H | protein phosphatase, Mg2+/Mn2+ dependent 1H(PPM1H) | 0.023824 |
| PPM1J | protein phosphatase, Mg2+/Mn2+ dependent 1J(PPM1J) | −0.41164 |
| PPM1K | protein phosphatase, Mg2+/Mn2+ dependent 1K(PPM1K) | −0.07317 |
| PPM1L | protein phosphatase, Mg2+/Mn2+ dependent 1L(PPM1L) | −0.38686 |
| PPM1M | protein phosphatase, Mg2+/Mn2+ dependent 1M(PPM1M) | −0.15775 |
| PPM1N | protein phosphatase, Mg2+/Mn2+ dependent 1N (putative)(PPM1N) | −0.25108 |
| PPOX | protoporphyrinogen oxidase(PPOX) | 0.22267 |
| PPP1CA | protein phosphatase 1 catalytic subunit alpha(PPP1CA) | 0.030585 |
| PPP1CB | protein phosphatase 1 catalytic subunit beta(PPP1CB) | −0.49178 |
| PPP1CC | protein phosphatase 1 catalytic subunit gamma(PPP1CC) | −0.17069 |
| PPP1R12A | protein phosphatase 1 regulatory subunit 12A(PPP1R12A) | 0.345004 |
| PPP1R15B | protein phosphatase 1 regulatory subunit 15B(PPP1R15B) | −0.40862 |
| PPP1R3C | protein phosphatase 1 regulatory subunit 3C(PPP1R3C) | −0.10075 |
| PPP1R3D | protein phosphatase 1 regulatory subunit 3D(PPP1R3D) | −0.11073 |
| PPP2CA | protein phosphatase 2 catalytic subunit alpha(PPP2CA) | −0.80331 |
| PPP2CB | protein phosphatase 2 catalytic subunit beta(PPP2CB) | −0.35563 |
| PPP2R1A | protein phosphatase 2 scaffold subunit Aalpha(PPP2R1A) | −0.45202 |
| PPP2R2A | protein phosphatase 2 regulatory subunit Balpha(PPP2R2A) | −0.15682 |
| PPP2R3B | protein phosphatase 2 regulatory subunit B″beta(PPP2R3B) | 0.004376 |
| PPP3CA | protein phosphatase 3 catalytic subunit alpha(PPP3CA) | −0.14881 |
| PPP3CB | protein phosphatase 3 catalytic subunit beta(PPP3CB) | −0.12011 |
| PPP3CC | protein phosphatase 3 catalytic subunit gamma(PPP3CC) | −0.27748 |
| PPP3R1 | protein phosphatase 3 regulatory subunit B, alpha(PPP3R1) | −0.0847 |
| PPP4C | protein phosphatase 4 catalytic subunit(PPP4C) | −0.1161 |
| PPP4R1 | protein phosphatase 4 regulatory subunit 1(PPP4R1) | −0.36669 |
| PPP5C | protein phosphatase 5 catalytic subunit(PPP5C) | −0.16858 |
| PPP6C | protein phosphatase 6 catalytic subunit(PPP6C) | −0.79169 |
| PPT1 | palmitoyl-protein thioesterase 1(PPT1) | 0.11659 |
| PPT2 | palmitoyl-protein thioesterase 2(PPT2) | −0.09181 |
| PPTC7 | PTC7 protein phosphatase homolog(PPTC7) | −0.26316 |
| PRDX1 | peroxiredoxin 1(PRDX1) | 0.131081 |
| PRDX2 | peroxiredoxin 2(PRDX2) | −0.6252 |
| PRDX3 | peroxiredoxin 3(PRDX3) | −0.29384 |
| PRDX4 | peroxiredoxin 4(PRDX4) | −0.30803 |
| PRDX5 | peroxiredoxin 5(PRDX5) | −0.12065 |
| PRDX6 | peroxiredoxin 6(PRDX6) | −0.03118 |
| PRKAA1 | protein kinase AMP-activated catalytic subunit alpha 1(PRKAA1) | −0.36447 |
| PRKAA2 | protein kinase AMP-activated catalytic subunit alpha 2(PRKAA2) | 0.21499 |
| PRKAB1 | protein kinase AMP-activated non-catalytic subunit beta 1(PRKAB1) | 0.070142 |
| PRKAB2 | protein kinase AMP-activated non-catalytic subunit beta 2(PRKAB2) | 0.064893 |
| PRKACA | protein kinase cAMP-activated catalytic subunit alpha(PRKACA) | −0.36871 |
| PRKACB | protein kinase cAMP-activated catalytic subunit beta(PRKACB) | −0.24808 |
| PRKAG1 | protein kinase AMP-activated non-catalytic subunit gamma 1(PRKAG1) | 0.27628 |
| PRKAG2 | protein kinase AMP-activated non-catalytic subunit gamma 2(PRKAG2) | −0.13985 |
| PRKAG3 | protein kinase AMP-activated non-catalytic subunit gamma 3(PRKAG3) | −0.19855 |
| PRODH | proline dehydrogenase 1(PRODH) | −0.23787 |
| PRODH2 | proline dehydrogenase 2(PRODH2) | −0.21312 |
| PRPS1 | phosphoribosyl pyrophosphate synthetase 1(PRPS1) | −0.03309 |
| PRPS1L1 | phosphoribosyl pyrophosphate synthetase 1-like 1(PRPS1L1) | −0.10212 |
| PRPS2 | phosphoribosyl pyrophosphate synthetase 2(PRPS2) | −0.07994 |
| PRPSAP1 | phosphoribosyl pyrophosphate synthetase associated protein 1(PRPSAP1) | −0.17825 |
| PRPSAP2 | phosphoribosyl pyrophosphate synthetase associated protein 2(PRPSAP2) | −0.12627 |
| PSAT1 | phosphoserine aminotransferase 1(PSAT1) | −0.3853 |
| PSMD11 | proteasome 26S subunit, non-ATPase 11(PSMD11) | 0.026795 |
| PSMD7 | proteasome 26S subunit, non-ATPase 7(PSMD7) | −0.18394 |
| PSPH | phosphoserine phosphatase(PSPH) | −0.13584 |
| PTDSS1 | phosphatidylserine synthase 1(PTDSS1) | −0.45211 |
| PTEN | phosphatase and tensin homolog(PTEN) | −0.03661 |
| PTGDS | prostaglandin D2 synthase(PTGDS) | −0.3874 |
| PTGER3 | prostaglandin E receptor 3(PTGER3) | −0.26142 |
| PTGES | prostaglandin E synthase(PTGES) | −0.04615 |
| PTGES3 | prostaglandin E synthase 3(PTGES3) | −0.17463 |
| PTGIS | prostaglandin 12 synthase(PTGIS) | −0.06612 |
| PTGR1 | prostaglandin reductase 1(PTGR1) | 0.185592 |
| PTGR2 | prostaglandin reductase 2(PTGR2) | −0.11283 |
| PTGS1 | prostaglandin-endoperoxide synthase 1(PTGS1) | −0.00255 |
| PTGS2 | prostaglandin-endoperoxide synthase 2(PTGS2) | −0.27614 |
| PTP4A1 | protein tyrosine phosphatase type IVA, member 1(PTP4A1) | −0.11578 |
| PTP4A2 | protein tyrosine phosphatase type IVA, member 2(PTP4A2) | 0.024294 |
| PTP4A3 | protein tyrosine phosphatase type IVA, member 3(PTP4A3) | −0.26783 |
| PTPDC1 | protein tyrosine phosphatase domain containing 1(PTPDC1) | −0.29099 |
| PTPLA | | −0.10386 |
| PTPMT1 | protein tyrosine phosphatase, mitochondrial 1(PTPMT1) | −0.30159 |
| PTPN1 | protein tyrosine phosphatase, non-receptor type 1(PTPN1) | −0.21496 |
| PTPN11 | protein tyrosine phosphatase, non-receptor type 11(PTPN11) | 0.022028 |
| PTPN12 | protein tyrosine phosphatase, non-receptor type 12(PTPN12) | −0.07224 |
| PTPN13 | protein tyrosine phosphatase, non-receptor type 13(PTPN13) | −0.16775 |
| PTPN14 | protein tyrosine phosphatase, non-receptor type 14(PTPN14) | −0.08753 |
| PTPN18 | protein tyrosine phosphatase, non-receptor type 18(PTPN18) | 0.005233 |
| PTPN2 | protein tyrosine phosphatase, non-receptor type 2(PTPN2) | −0.05354 |
| PTPN20A | | −0.04007 |
| PTPN20B | | −0.04007 |
| PTPN21 | protein tyrosine phosphatase, non-receptor type 21(PTPN21) | −0.11035 |
| PTPN22 | protein tyrosine phosphatase, non-receptor type 22(PTPN22) | −0.4256 |
| PTPN23 | protein tyrosine phosphatase, non-receptor type 23(PTPN23) | −0.25133 |
| PTPN3 | protein tyrosine phosphatase, non-receptor type 3(PTPN3) | 0.110292 |
| PTPN4 | protein tyrosine phosphatase, non-receptor type 4(PTPN4) | −0.25594 |
| PTPN5 | protein tyrosine phosphatase, non-receptor type 5(PTPN5) | −0.21487 |
| PTPN6 | protein tyrosine phosphatase, non-receptor type 6(PTPN6) | 0.22631 |
| PTPN7 | protein tyrosine phosphatase, non-receptor type 7(PTPN7) | −0.17986 |
| PTPN9 | protein tyrosine phosphatase, non-receptor type 9(PTPN9) | 0.146117 |
| PTPRA | protein tyrosine phosphatase, receptor type A(PTPRA) | −0.14245 |
| PTPRB | protein tyrosine phosphatase, receptor type B(PTPRB) | −0.16654 |
| PTPRC | protein tyrosine phosphatase, receptor type C(PTPRC) | −0.41036 |
| PTPRD | protein tyrosine phosphatase, receptor type D(PTPRD) | −0.55149 |
| PTPRE | protein tyrosine phosphatase, receptor type E(PTPRE) | −0.40145 |
| PTPRF | protein tyrosine phosphatase, receptor type F(PTPRF) | −0.19233 |
| PTPRG | protein tyrosine phosphatase, receptor type G(PTPRG) | −0.02179 |
| PTPRH | protein tyrosine phosphatase, receptor type H(PTPRH) | −0.40141 |
| PTPRJ | protein tyrosine phosphatase, receptor type J(PTPRJ) | −0.08434 |
| PTPRK | protein tyrosine phosphatase, receptor type K(PTPRK) | −0.06735 |
| PTPRM | protein tyrosine phosphatase, receptor type M(PTPRM) | −0.07976 |
| PTPRN | protein tyrosine phosphatase, receptor type N(PTPRN) | −0.2568 |
| PTPRN2 | protein tyrosine phosphatase, receptor type N2(PTPRN2) | −0.07504 |
| PTPRO | protein tyrosine phosphatase, receptor type O(PTPRO) | −0.2462 |
| PTPRR | protein tyrosine phosphatase, receptor type R(PTPRR) | −0.09703 |
| PTPRS | protein tyrosine phosphatase, receptor type S(PTPRS) | −0.27793 |
| PTPRT | protein tyrosine phosphatase, receptor type T(PTPRT) | −0.35797 |
| PTPRU | protein tyrosine phosphatase, receptor type U(PTPRU) | −0.269 |
| PTPRZ1 | protein tyrosine phosphatase, receptor type Z1(PTPRZ1) | −0.12236 |
| PTRHD1 | peptidyl-tRNA hydrolase domain containing 1(PTRHD1) | −0.03736 |
| PTS | 6-pyruvoyltetrahydropterin synthase(PTS) | −0.21985 |
| PYCR1 | pyrroline-5-carboxylate reductase 1(PYCR1) | −0.06354 |
| PYCR2 | pyrroline-5-carboxylate reductase family member 2(PYCR2) | 0.152729 |
| PYCRL | pyrroline-5-carboxylate reductase-like(PYCRL) | −0.27569 |
| PYGB | phosphorylase, glycogen; brain(PYGB) | 0.031496 |
| PYGL | phosphorylase, glycogen, liver(PYGL) | −0.27717 |
| PYGM | phosphorylase, glycogen, muscle(PYGM) | −0.20064 |
| PYROXD1 | pyridine nucleotide-disulphide oxidoreductase domain 1(PYROXD1) | 0.16043 |
| QARS | glutaminyl-tRNA synthetase(QARS) | −0.11251 |
| QDPR | quinoid dihydropteridine reductase(QDPR) | −0.25818 |
| RARA | retinoic acid receptor alpha(RARA) | −0.08995 |
| RARB | retinoic acid receptor beta(RARB) | 0.067182 |
| RARG | retinoic acid receptor gamma(RARG) | −0.11136 |
| RBKS | ribokinase(RBKS) | −0.14167 |
| RBM46 | RNA binding motif protein 46(RBM46) | 0.125818 |
| RBMY1B | RNA binding motif protein, Y-linked, family 1, member B(RBMY1B) | −0.18609 |
| RDH10 | retinol dehydrogenase 10 (all-trans)(RDH10) | −0.16955 |
| RDH11 | retinol dehydrogenase 11 (all-trans/9-cis/11-cis)(RDH11) | 0.211502 |
| RDH12 | retinol dehydrogenase 12 (all-trans/9-cis/11-cis)(RDH12) | −0.15465 |
| RDH13 | retinol dehydrogenase 13(RDH13) | −0.21375 |
| RDH14 | retinol dehydrogenase 14 (all-trans/9-cis/11-cis)(RDH14) | −0.09156 |
| RDH16 | retinol dehydrogenase 16 (all-trans)(RDH16) | −0.01692 |
| RDH5 | retinol dehydrogenase 5(RDH5) | 0.149653 |
| RDH8 | retinol dehydrogenase 8 (all-trans)(RDH8) | −0.00202 |
| RENBP | renin binding protein(RENBP) | −0.19835 |
| RFK | riboflavin kinase(RFK) | −0.55731 |
| RHAG | Rh-associated glycoprotein(RHAG) | −0.42334 |
| RHCG | Rh family C glycoprotein(RHCG) | −0.51102 |
| RILPL2 | Rab interacting lysosomal protein like 2(RILPL2) | −0.37673 |
| RNGTT | RNA guanylyltransferase and 5′-phosphatase(RNGTT) | −0.4308 |
| RORA | RAR related orphan receptor A(RORA) | −0.18716 |
| RORB | RAR related orphan receptor B(RORB) | −0.15348 |
| RORC | RAR related orphan receptor C(RORC) | −0.09828 |
| RPA2 | replication protein A2(RPA2) | −0.62652 |
| RPAP2 | RNA polymerase II associated protein 2(RPAP2) | −0.09269 |
| RPE | ribulose-5-phosphate-3-epimerase(RPE) | −0.21186 |
| RPE65 | RPE65, retinoid isomerohydrolase(RPE65) | −0.30017 |
| RPIA | ribose 5-phosphate isomerase A(RPIA) | −0.19853 |
| RPL11 | ribosomal protein L11(RPL11) | −1.37662 |
| RPL12 | ribosomal protein L12(RPL12) | −0.32949 |
| RPL13 | ribosomal protein L13(RPL13) | −0.25828 |
| RPL14 | ribosomal protein L14(RPL14) | −0.61733 |
| RPL18A | ribosomal protein L18a(RPL18A) | −0.06099 |
| RPL19 | ribosomal protein L19(RPL19) | −0.09525 |
| RPL23A | ribosomal protein L23a(RPL23A) | −0.6716 |
| RPL26 | ribosomal protein L26(RPL26) | −0.48626 |
| RPL27 | ribosomal protein L27(RPL27) | 0.050205 |
| RPL3 | ribosomal protein L3(RPL3) | −0.20557 |
| RPL30 | ribosomal protein L30(RPL30) | −0.53536 |
| RPL31 | ribosomal protein L31(RPL31) | −0.40745 |
| RPL32 | ribosomal protein L32(RPL32) | −1.08727 |
| RPL34 | ribosomal protein L34(RPL34) | −0.94071 |
| RPL35 | ribosomal protein L35(RPL35) | −0.8599 |
| RPL35A | ribosomal protein L35a(RPL35A) | −0.47801 |
| RPL36 | ribosomal protein L36(RPL36) | −0.37375 |
| RPL37 | ribosomal protein L37(RPL37) | −0.64123 |
| RPL37A | ribosomal protein L37a(RPL37A) | −0.03976 |
| RPL38 | ribosomal protein L38(RPL38) | −0.25857 |
| RPL4 | ribosomal protein L4(RPL4) | −0.73763 |
| RPL6 | ribosomal protein L6(RPL6) | −0.2553 |
| RPL7 | ribosomal protein L7(RPL7) | −0.39018 |
| RPL7A | ribosomal protein L7a(RPL7A) | −0.28703 |
| RPLP2 | ribosomal protein lateral stalk subunit P2(RPLP2) | −0.12897 |
| RPN2 | ribophorin II(RPN2) | −0.09611 |
| RPS11 | ribosomal protein S11(RPS11) | 0.542702 |
| RPS13 | ribosomal protein S13(RPS13) | −0.45708 |
| RPS14 | ribosomal protein S14(RPS14) | −0.2143 |
| RPS15 | ribosomal protein S15(RPS15) | −0.4262 |
| RPS17 | ribosomal protein S17(RPS17) | −0.10006 |
| RPS18 | ribosomal protein S18(RPS18) | −0.00019 |
| RPS19 | ribosomal protein S19(RPS19) | −0.22432 |
| RPS24 | ribosomal protein S24(RPS24) | 0.182804 |
| RPS27A | ribosomal protein S27a(RPS27A) | −0.4113 |
| RPS3 | ribosomal protein S3(RPS3) | −0.16196 |
| RPS4X | ribosomal protein S4, X-linked(RPS4X) | −0.39369 |
| RPS5 | ribosomal protein S5(RPS5) | −0.13889 |
| RPS6 | ribosomal protein S6(RPS6) | −0.21109 |
| RPS7 | ribosomal protein S7(RPS7) | 0.195066 |
| RPS8 | ribosomal protein S8(RPS8) | −1.00065 |
| RPSA | ribosomal protein SA(RPSA) | −0.18522 |
| RRM1 | ribonucleotide reductase catalytic subunit M1(RRM1) | 0.356778 |
| RRM2 | ribonucleotide reductase regulatory subunit M2(RRM2) | −1.13772 |
| RRM2B | ribonucleotide reductase regulatory TP53 inducible subunit M2B(RRM2B) | −0.32849 |
| RS1 | retinoschisin 1(RS1) | −0.28434 |
| RUVBL2 | RuvB like AAA ATPase 2(RUVBL2) | −0.12453 |
| RXRA | retinoid X receptor alpha(RXRA) | 0.10232 |
| RXRB | retinoid X receptor beta(RXRB) | 0.006825 |
| RXRG | retinoid X receptor gamma(RXRG) | −0.08336 |
| RYR1 | ryanodine receptor 1(RYR1) | −0.0337 |
| RYR2 | ryanodine receptor 2(RYR2) | −0.50488 |
| RYR3 | ryanodine receptor 3(RYR3) | −0.04623 |
| S100G | S100 calcium binding protein G(S100G) | −0.1598 |
| SARDH | sarcosine dehydrogenase(SARDH) | −0.225 |
| SBF1 | SET binding factor 1(SBF1) | −0.51313 |
| SC5D | sterol-C5-desaturase(SC5D) | −0.1529 |
| SCCPDH | saccharopine dehydrogenase (putative)(SCCPDH) | −0.26165 |
| SCD | stearoyl-CoA desaturase(SCD) | −0.43861 |
| SCD5 | stearoyl-CoA desaturase 5(SCD5) | −0.15499 |
| SCLY | selenocysteine lyase(SCLY) | 0.08325 |
| SCN10A | sodium voltage-gated channel alpha subunit 10(SCN10A) | −0.38677 |
| SCN11A | sodium voltage-gated channel alpha subunit 11(SCN11A) | −0.28762 |
| SCN1A | sodium voltage-gated channel alpha subunit 1(SCN1A) | −0.09741 |
| SCN1B | sodium voltage-gated channel beta subunit 1(SCN1B) | −0.12301 |
| SCN2A | sodium voltage-gated channel alpha subunit 2(SCN2A) | −0.028 |
| SCN2B | sodium voltage-gated channel beta subunit 2(SCN2B) | −0.42731 |
| SCN3A | sodium voltage-gated channel alpha subunit 3(SCN3A) | −0.23998 |
| SCN3B | sodium voltage-gated channel beta subunit 3(SCN3B) | 0.007142 |
| SCN4A | sodium voltage-gated channel alpha subunit 4(SCN4A) | −0.1693 |
| SCN5A | sodium voltage-gated channel alpha subunit 5(SCN5A) | −0.10934 |
| SCN7A | sodium voltage-gated channel alpha subunit 7(SCN7A) | −0.22053 |
| SCN8A | sodium voltage-gated channel alpha subunit 8(SCN8A) | −0.10145 |
| SCN9A | sodium voltage-gated channel alpha subunit 9(SCN9A) | −0.1157 |
| SCNN1A | sodium channel epithelial 1 alpha subunit(SCNN1A) | −0.30261 |
| SCNN1B | sodium channel epithelial 1 beta subunit(SCNN1B) | 0.001256 |
| SCNN1D | sodium channel epithelial 1 delta subunit(SCNN1D) | −0.03858 |
| SCNN1G | sodium channel epithelial 1 gamma subunit(SCNN1G) | 0.153596 |
| SCO1 | SCO1, cytochrome c oxidase assembly protein(SCO1) | −0.38529 |
| SCO2 | SCO2, cytochrome c oxidase assembly protein(SCO2) | −0.15516 |
| SCP2 | sterol carrier protein 2(SCP2) | −0.33998 |
| SDAD1 | SDA1 domain containing 1(SDAD1) | −0.08425 |
| SDHA | succinate dehydrogenase complex flavoprotein subunit A(SDHA) | −0.67347 |
| SDHB | succinate dehydrogenase complex iron sulfur subunit B(SDHB) | −0.12929 |
| SDHC | succinate dehydrogenase complex subunit C(SDHC) | −0.85023 |
| SDHD | succinate dehydrogenase complex subunit D(SDHD) | −0.05701 |
| SDR16C5 | short chain dehydrogenase/reductase family 16C, member 5(SDR16C5) | −0.14673 |
| SDR39U1 | short chain dehydrogenase/reductase family 39U member 1(SDR39U1) | −0.11159 |
| SDR42E1 | short chain dehydrogenase/reductase family 42E, member 1(SDR42E1) | −0.89395 |
| SDR9C7 | short chain dehydrogenase/reductase family 9C, member 7(SDR9C7) | −0.21235 |
| SDS | serine dehydratase(SDS) | −0.12912 |
| SDSL | serine dehydratase like(SDSL) | −0.1011 |
| SEPHS1 | selenophosphate synthetase 1(SEPHS1) | −0.38027 |
| SEPHS2 | selenophosphate synthetase 2(SEPHS2) | −0.1604 |
| SF1 | splicing factor 1(SF1) | −0.02365 |
| SF3A1 | splicing factor 3a subunit 1(SF3A1) | −1.18088 |
| SF3A2 | splicing factor 3a subunit 2(SF3A2) | −0.16438 |
| SF3B1 | splicing factor 3b subunit 1(SF3B1) | #NAME? |
| SF3B2 | splicing factor 3b subunit 2(SF3B2) | 0.005258 |
| SF3B3 | splicing factor 3b subunit 3(SF3B3) | −0.79188 |
| SF3B4 | splicing factor 3b subunit 4(SF3B4) | −0.28583 |
| SF3B5 | splicing factor 3b subunit 5(SF3B5) | −0.68287 |
| SFXN5 | sideroflexin 5(SFXN5) | 0.155047 |
| SGMS1 | sphingomyelin synthase 1(SGMS1) | 0.104341 |
| SGMS2 | sphingomyelin synthase 2(SGMS2) | 0.034398 |
| SGPL1 | sphingosine-1-phosphate lyase 1(SGPL1) | 0.00238 |
| SGPP1 | sphingosine-1-phosphate phosphatase 1(SGPP1) | −0.12135 |
| SGPP2 | sphingosine-1-phosphate phosphatase 2(SGPP2) | −0.10146 |
| SGSH | N-sulfoglucosamine sulfohydrolase(SGSH) | −0.11991 |
| SHMT1 | serine hydroxymethyltransferase 1(SHMT1) | −0.00026 |
| SHMT2 | serine hydroxymethyltransferase 2(SHMT2) | −0.35676 |
| SHOX | short stature homeobox(SHOX) | −0.10015 |
| SI | sucrase-isomaltase(SI) | 0.28753 |
| SIAE | sialic acid acetylesterase(SIAE) | −0.28596 |
| SIGLECL1 | SIGLEC family like 1(SIGLECL1) | −0.25629 |
| SLC10A1 | solute carrier family 10 member 1(SLC10A1) | −0.16684 |
| SLC10A2 | solute carrier family 10 member 2(SLC10A2) | 0.031573 |
| SLC10A3 | solute carrier family 10 member 3(SLC10A3) | −0.49426 |
| SLC10A4 | solute carrier family 10 member 4(SLC10A4) | −0.1131 |
| SLC10A5 | solute carrier family 10 member 5(SLC10A5) | −0.12699 |
| SLC10A6 | solute carrier family 10 member 6(SLC10A6) | −0.18721 |
| SLC11A1 | solute carrier family 11 member 1(SLC11A1) | −0.42483 |
| SLC11A2 | solute carrier family 11 member 2(SLC11A2) | −0.79828 |
| SLC12A1 | solute carrier family 12 member 1(SLC12A1) | −0.29505 |
| SLC12A2 | solute carrier family 12 member 2(SLC12A2) | −0.04105 |
| SLC12A3 | solute carrier family 12 member 3(SLC12A3) | −0.43288 |
| SLC12A4 | solute carrier family 12 member 4(SLC12A4) | −0.13942 |
| SLC12A5 | solute carrier family 12 member 5(SLC12A5) | 0.113383 |
| SLC12A6 | solute carrier family 12 member 6(SLC12A6) | 0.040641 |
| SLC12A7 | solute carrier family 12 member 7(SLC12A7) | −0.24782 |
| SLC12A8 | solute carrier family 12 member 8(SLC12A8) | −0.21954 |
| SLC12A9 | solute carrier family 12 member 9(SLC12A9) | −0.16517 |
| SLC13A3 | solute carrier family 13 member 3(SLC13A3) | −0.15373 |
| SLC14A1 | solute carrier family 14 member 1 (Kidd blood group)(SLC14A1) | −0.0876 |
| SLC14A2 | solute carrier family 14 member 2(SLC14A2) | −0.46405 |
| SLC15A1 | solute carrier family 15 member 1(SLC15A1) | −0.05098 |
| SLC15A2 | solute carrier family 15 member 2(SLC15A2) | −0.3115 |
| SLC15A3 | solute carrier family 15 member 3(SLC15A3) | −0.38106 |
| SLC15A4 | solute carrier family 15 member 4(SLC15A4) | 0.120363 |
| SLC16A1 | solute carrier family 16 member 1(SLC16A1) | −0.24119 |
| SLC16A10 | solute carrier family 16 member 10(SLC16A10) | 0.29646 |
| SLC16A11 | solute carrier family 16 member 11(SLC16A11) | 0.30641 |
| SLC16A12 | solute carrier family 16 member 12(SLC16A12) | 0.0096 |
| SLC16A13 | solute carrier family 16 member 13(SLC16A13) | −0.19579 |
| SLC16A14 | solute carrier family 16 member 14(SLC16A14) | −0.20096 |
| SLC16A2 | solute carrier family 16 member 2(SLC16A2) | −0.27594 |
| SLC16A3 | solute carrier family 16 member 3(SLC16A3) | −0.30961 |
| SLC16A4 | solute carrier family 16 member 4(SLC16A4) | −0.10697 |
| SLC16A5 | solute carrier family 16 member 5(SLC16A5) | −0.27182 |
| SLC16A6 | solute carrier family 16 member 6(SLC16A6) | −0.05513 |
| SLC16A7 | solute carrier family 16 member 7(SLC16A7) | −0.1246 |
| SLC16A8 | solute carrier family 16 member 8(SLC16A8) | −0.14281 |
| SLC16A9 | solute carrier family 16 member 9(SLC16A9) | −0.12396 |
| SLC17A1 | solute carrier family 17 member 1(SLC17A1) | −0.27308 |
| SLC17A2 | solute carrier family 17 member 2(SLC17A2) | −0.1718 |
| SLC17A3 | solute carrier family 17 member 3(SLC17A3) | 0.243622 |
| SLC17A4 | solute carrier family 17 member 4(SLC17A4) | 0.1156 |
| SLC17A5 | solute carrier family 17 member 5(SLC17A5) | −0.33591 |
| SLC17A6 | solute carrier family 17 member 6(SLC17A6) | −0.05168 |
| SLC17A7 | solute carrier family 17 member 7(SLC17A7) | −0.27687 |
| SLC17A8 | solute carrier family 17 member 8(SLC17A8) | −0.09503 |
| SLC18A1 | solute carrier family 18 member A1(SLC18A1) | −0.40597 |
| SLC18A2 | solute carrier family 18 member A2(SLC18A2) | −0.32496 |
| SLC18A3 | solute carrier family 18 member A3(SLC18A3) | 0.034397 |
| SLC19A2 | solute carrier family 19 member 2(SLC19A2) | −0.25795 |
| SLC19A3 | solute carrier family 19 member 3(SLC19A3) | −0.14157 |
| SLC1A1 | solute carrier family 1 member 1(SLC1A1) | 0.00106 |
| SLC1A3 | solute carrier family 1 member 3(SLC1A3) | −0.16247 |
| SLC1A4 | solute carrier family 1 member 4(SLC1A4) | 0.29467 |
| SLC1A5 | solute carrier family 1 member 5(SLC1A5) | 0.53873 |
| SLC20A1 | solute carrier family 20 member 1(SLC20A1) | 0.239091 |
| SLC20A2 | solute carrier family 20 member 2(SLC20A2) | −0.50997 |
| SLC22A1 | solute carrier family 22 member 1(SLC22A1) | −0.09005 |
| SLC22A11 | solute carrier family 22 member 11(SLC22A11) | −0.13456 |
| SLC22A12 | solute carrier family 22 member 12(SLC22A12) | −0.23591 |
| SLC22A13 | solute carrier family 22 member 13(SLC22A13) | −0.05022 |
| SLC22A14 | solute carrier family 22 member 14(SLC22A14) | 0.00506 |
| SLC22A15 | solute carrier family 22 member 15(SLC22A15) | −0.04087 |
| SLC22A16 | solute carrier family 22 member 16(SLC22A16) | −0.29991 |
| SLC22A17 | solute carrier family 22 member 17(SLC22A17) | −0.23367 |
| SLC22A18 | solute carrier family 22 member 18(SLC22A18) | −0.4868 |
| SLC22A2 | solute carrier family 22 member 2(SLC22A2) | −0.05338 |
| SLC22A3 | solute carrier family 22 member 3(SLC22A3) | −0.09442 |
| SLC22A4 | solute carrier family 22 member 4(SLC22A4) | −0.3244 |
| SLC22A5 | solute carrier family 22 member 5(SLC22A5) | 0.39124 |
| SLC22A6 | solute carrier family 22 member 6(SLC22A6) | −0.04293 |
| SLC22A7 | solute carrier family 22 member 7(SLC22A7) | −0.36655 |
| SLC22A8 | solute carrier family 22 member 8(SLC22A8) | −0.84398 |
| SLC22A9 | solute carrier family 22 member 9(SLC22A9) | −0.16033 |
| SLC24A1 | solute carrier family 24 member 1(SLC24A1) | 0.062595 |
| SLC24A2 | solute carrier family 24 member 2(SLC24A2) | −0.30566 |
| SLC24A3 | solute carrier family 24 member 3(SLC24A3) | −0.15108 |
| SLC24A4 | solute carrier family 24 member 4(SLC24A4) | −0.62865 |
| SLC24A5 | solute carrier family 24 member 5(SLC24A5) | 0.127474 |
| SLC25A1 | solute carrier family 25 member 1(SLC25A1) | 0.506875 |
| SLC25A10 | solute carrier family 25 member 10(SLC25A10) | −0.09047 |
| SLC25A11 | solute carrier family 25 member 11(SLC25A11) | −0.14889 |
| SLC25A12 | solute carrier family 25 member 12(SLC25A12) | −0.36914 |
| SLC25A13 | solute carrier family 25 member 13(SLC25A13) | −0.23368 |
| SLC25A14 | solute carrier family 25 member 14(SLC25A14) | −0.19822 |
| SLC25A15 | solute carrier family 25 member 15(SLC25A15) | −0.28958 |
| SLC25A16 | solute carrier family 25 member 16(SLC25A16) | 0.020759 |
| SLC25A17 | solute carrier family 25 member 17(SLC25A17) | −0.06748 |
| SLC25A18 | solute carrier family 25 member 18(SLC25A18) | −0.03762 |
| SLC25A19 | solute carrier family 25 member 19(SLC25A19) | −0.52224 |
| SLC25A2 | solute carrier family 25 member 2(SLC25A2) | −0.30557 |
| SLC25A20 | solute carrier family 25 member 20(SLC25A20) | −0.2521 |
| SLC25A21 | solute carrier family 25 member 21(SLC25A21) | −0.04103 |
| SLC25A22 | solute carrier family 25 member 22(SLC25A22) | 0.143635 |
| SLC25A23 | solute carrier family 25 member 23(SLC25A23) | −0.15028 |
| SLC25A24 | solute carrier family 25 member 24(SLC25A24) | −0.43084 |
| SLC25A25 | solute carrier family 25 member 25(SLC25A25) | −0.26349 |
| SLC25A26 | solute carrier family 25 member 26(SLC25A26) | −0.42952 |
| SLC25A27 | solute carrier family 25 member 27(SLC25A27) | −0.03103 |
| SLC25A28 | solute carrier family 25 member 28(SLC25A28) | −0.18036 |
| SLC25A29 | solute carrier family 25 member 29(SLC25A29) | −0.01989 |
| SLC25A3 | solute carrier family 25 member 3(SLC25A3) | −0.03682 |
| SLC25A30 | solute carrier family 25 member 30(SLC25A30) | −0.32275 |
| SLC25A31 | solute carrier family 25 member 31(SLC25A31) | −0.2946 |
| SLC25A32 | solute carrier family 25 member 32(SLC25A32) | −0.3238 |
| SLC25A33 | solute carrier family 25 member 33(SLC25A33) | −0.19308 |
| SLC25A34 | solute carrier family 25 member 34(SLC25A34) | −0.34772 |
| SLC25A35 | solute carrier family 25 member 35(SLC25A35) | −0.11075 |
| SLC25A36 | solute carrier family 25 member 36(SLC25A36) | −0.20317 |
| SLC25A37 | solute carrier family 25 member 37(SLC25A37) | −0.33398 |
| SLC25A38 | solute carrier family 25 member 38(SLC25A38) | −0.23169 |
| SLC25A39 | solute carrier family 25 member 39(SLC25A39) | −0.20274 |
| SLC25A4 | solute carrier family 25 member 4(SLC25A4) | −0.01776 |
| SLC25A40 | solute carrier family 25 member 40(SLC25A40) | −0.3651 |
| SLC25A41 | solute carrier family 25 member 41(SLC25A41) | −0.11012 |
| SLC25A42 | solute carrier family 25 member 42(SLC25A42) | −0.28829 |
| SLC25A43 | solute carrier family 25 member 43(SLC25A43) | 0.013067 |
| SLC25A44 | solute carrier family 25 member 44(SLC25A44) | −0.31366 |
| SLC25A45 | solute carrier family 25 member 45(SLC25A45) | 0.07377 |
| SLC25A46 | solute carrier family 25 member 46(SLC25A46) | −0.29556 |
| SLC25A5 | solute carrier family 25 member 5(SLC25A5) | −0.17163 |
| SLC25A6 | solute carrier family 25 member 6(SLC25A6) | −0.16553 |
| SLC26A2 | solute carrier family 26 member 2(SLC26A2) | −0.57731 |
| SLC26A3 | solute carrier family 26 member 3(SLC26A3) | −0.24125 |
| SLC26A4 | solute carrier family 26 member 4(SLC26A4) | −0.36675 |
| SLC27A1 | solute carrier family 27 member 1(SLC27A1) | −0.28073 |
| SLC27A2 | solute carrier family 27 member 2(SLC27A2) | −0.61923 |
| SLC27A3 | solute carrier family 27 member 3(SLC27A3) | −0.0451 |
| SLC27A4 | solute carrier family 27 member 4(SLC27A4) | −0.21816 |
| SLC27A5 | solute carrier family 27 member 5(SLC27A5) | −0.46101 |
| SLC27A6 | solute carrier family 27 member 6(SLC27A6) | −0.33776 |
| SLC2A1 | solute carrier family 2 member 1(SLC2A1) | −0.67894 |
| SLC2A10 | solute carrier family 2 member 10(SLC2A10) | −0.43582 |
| SLC2A11 | solute carrier family 2 member 11(SLC2A11) | 0.044539 |
| SLC2A12 | solute carrier family 2 member 12(SLC2A12) | −0.35416 |
| SLC2A13 | solute carrier family 2 member 13(SLC2A13) | −0.29828 |
| SLC2A14 | solute carrier family 2 member 14(SLC2A14) | −0.21277 |
| SLC2A2 | solute carrier family 2 member 2(SLC2A2) | −0.12389 |
| SLC2A3 | solute carrier family 2 member 3(SLC2A3) | −0.10692 |
| SLC2A4 | solute carrier family 2 member 4(SLC2A4) | −0.22429 |
| SLC2A5 | solute carrier family 2 member 5(SLC2A5) | −0.17237 |
| SLC2A6 | solute carrier family 2 member 6(SLC2A6) | −0.33023 |
| SLC2A7 | solute carrier family 2 member 7(SLC2A7) | −0.14803 |
| SLC2A8 | solute carrier family 2 member 8(SLC2A8) | −0.27839 |
| SLC2A9 | solute carrier family 2 member 9(SLC2A9) | −0.27267 |
| SLC30A3 | solute carrier family 30 member 3(SLC30A3) | −0.37666 |
| SLC34A2 | solute carrier family 34 member 2(SLC34A2) | −0.04537 |
| SLC35A2 | solute carrier family 35 member A2(SLC35A2) | −0.13619 |
| SLC35B2 | solute carrier family 35 member B2(SLC35B2) | −0.44049 |
| SLC35C2 | solute carrier family 35 member C2(SLC35C2) | −0.22093 |
| SLC35F2 | solute carrier family 35 member F2(SLC35F2) | −0.14039 |
| SLC37A1 | solute carrier family 37 member 1(SLC37A1) | −0.20781 |
| SLC37A2 | solute carrier family 37 member 2(SLC37A2) | −0.19999 |
| SLC37A3 | solute carrier family 37 member 3(SLC37A3) | −0.05165 |
| SLC38A1 | solute carrier family 38 member 1(SLC38A1) | −0.25788 |
| SLC38A2 | solute carrier family 38 member 2(SLC38A2) | 0.427993 |
| SLC39A4 | solute carrier family 39 member 4(SLC39A4) | −0.17654 |
| SLC39A6 | solute carrier family 39 member 6(SLC39A6) | −0.28629 |
| SLC3A1 | solute carrier family 3 member 1(SLC3A1) | −0.26349 |
| SLC3A2 | solute carrier family 3 member 2(SLC3A2) | −0.00597 |
| SLC40A1 | solute carrier family 40 member 1(SLC40A1) | −0.0718 |
| SLC43A1 | solute carrier family 43 member 1(SLC43A1) | 0.060477 |
| SLC43A2 | solute carrier family 43 member 2(SLC43A2) | −0.06679 |
| SLC43A3 | solute carrier family 43 member 3(SLC43A3) | −0.45359 |
| SLC44A1 | solute carrier family 44 member 1(SLC44A1) | 0.04657 |
| SLC45A2 | solute carrier family 45 member 2(SLC45A2) | −0.21917 |
| SLC45A3 | solute carrier family 45 member 3(SLC45A3) | −0.41785 |
| SLC46A2 | solute carrier family 46 member 2(SLC46A2) | −0.22515 |
| SLC46A3 | solute carrier family 46 member 3(SLC46A3) | −0.47389 |
| SLC4A1 | solute carrier family 4 member 1 (Diego blood group)(SLC4A1) | −0.22287 |
| SLC4A10 | solute carrier family 4 member 10(SLC4A10) | 0.045193 |
| SLC4A11 | solute carrier family 4 member 11(SLC4A11) | −0.28461 |
| SLC4A1AP | solute carrier family 4 member 1 adaptor protein(SLC4A1AP) | −0.11238 |
| SLC4A2 | solute carrier family 4 member 2(SLC4A2) | 0.020827 |
| SLC4A3 | solute carrier family 4 member 3(SLC4A3) | −0.35169 |
| SLC4A4 | solute carrier family 4 member 4(SLC4A4) | −0.02456 |
| SLC4A5 | solute carrier family 4 member 5(SLC4A5) | −0.39376 |
| SLC4A7 | solute carrier family 4 member 7(SLC4A7) | −0.53014 |
| SLC4A8 | solute carrier family 4 member 8(SLC4A8) | −0.08215 |
| SLC4A9 | solute carrier family 4 member 9(SLC4A9) | −0.06954 |
| SLC5A1 | solute carrier family 5 member 1(SLC5A1) | 0.16955 |
| SLC5A10 | solute carrier family 5 member 10(SLC5A10) | −0.0631 |
| SLC5A11 | solute carrier family 5 member 11(SLC5A11) | 0.063448 |
| SLC5A12 | solute carrier family 5 member 12(SLC5A12) | −0.06834 |
| SLC5A2 | solute carrier family 5 member 2(SLC5A2) | −0.21222 |
| SLC5A4 | solute carrier family 5 member 4(SLC5A4) | −0.03688 |
| SLC5A5 | solute carrier family 5 member 5(SLC5A5) | −0.43001 |
| SLC5A6 | solute carrier family 5 member 6(SLC5A6) | −0.12507 |
| SLC5A7 | solute carrier family 5 member 7(SLC5A7) | −0.09922 |
| SLC5A9 | solute carrier family 5 member 9(SLC5A9) | 0.207746 |
| SLC6A1 | solute carrier family 6 member 1(SLC6A1) | −0.08903 |
| SLC6A11 | solute carrier family 6 member 11(SLC6A11) | −0.2184 |
| SLC6A12 | solute carrier family 6 member 12(SLC6A12) | −0.16522 |
| SLC6A13 | solute carrier family 6 member 13(SLC6A13) | −0.04572 |
| SLC6A14 | solute carrier family 6 member 14(SLC6A14) | −0.15927 |
| SLC6A15 | solute carrier family 6 member 15(SLC6A15) | 0.02519 |
| SLC6A16 | solute carrier family 6 member 16(SLC6A16) | −0.53847 |
| SLC6A17 | solute carrier family 6 member 17(SLC6A17) | −0.25807 |
| SLC6A18 | solute carrier family 6 member 18(SLC6A18) | −0.16067 |
| SLC6A19 | solute carrier family 6 member 19(SLC6A19) | −0.09597 |
| SLC6A2 | solute carrier family 6 member 2(SLC6A2) | −0.21323 |
| SLC6A20 | solute carrier family 6 member 20(SLC6A20) | 0.13466 |
| SLC6A3 | solute carrier family 6 member 3(SLC6A3) | −0.00443 |
| SLC6A4 | solute carrier family 6 member 4(SLC6A4) | 0.06631 |
| SLC6A5 | solute carrier family 6 member 5(SLC6A5) | −0.1473 |
| SLC6A6 | solute carrier family 6 member 6(SLC6A6) | −0.1264 |
| SLC6A7 | solute carrier family 6 member 7(SLC6A7) | −0.60229 |
| SLC6A8 | solute carrier family 6 member 8(SLC6A8) | −0.33157 |
| SLC6A9 | solute carrier family 6 member 9(SLC6A9) | −0.1482 |
| SLC7A1 | solute carrier family 7 member 1(SLC7A1) | −0.19686 |
| SLC7A10 | solute carrier family 7 member 10(SLC7A10) | −0.52473 |
| SLC7A11 | solute carrier family 7 member 11(SLC7A11) | −0.04347 |
| SLC7A13 | solute carrier family 7 member 13(SLC7A13) | −0.47068 |
| SLC7A14 | solute carrier family 7 member 14(SLC7A14) | −0.37877 |
| SLC7A2 | solute carrier family 7 member 2(SLC7A2) | −0.2716 |
| SLC7A3 | solute carrier family 7 member 3(SLC7A3) | −0.09044 |
| SLC7A4 | solute carrier family 7 member 4(SLC7A4) | 0.20701 |
| SLC7A5 | solute carrier family 7 member 5(SLC7A5) | −0.25765 |
| SLC7A6 | solute carrier family 7 member 6(SLC7A6) | −0.43658 |
| SLC7A7 | solute carrier family 7 member 7(SLC7A7) | 0.040208 |
| SLC7A8 | solute carrier family 7 member 8(SLC7A8) | −0.14565 |
| SLC7A9 | solute carrier family 7 member 9(SLC7A9) | −0.28408 |
| SLC8A1 | solute carrier family 8 member A1(SLC8A1) | −0.18663 |
| SLC9A1 | solute carrier family 9 member A1(SLC9A1) | −0.06095 |
| SLC9A2 | solute carrier family 9 member A2(SLC9A2) | −0.37481 |
| SLC9A3 | solute carrier family 9 member A3(SLC9A3) | −0.24111 |
| SLC9A4 | solute carrier family 9 member A4(SLC9A4) | −0.19731 |
| SLC9A5 | solute carrier family 9 member A5(SLC9A5) | −0.35968 |
| SLC9A6 | solute carrier family 9 member A6(SLC9A6) | −0.16231 |
| SLC9A7 | solute carrier family 9 member A7(SLC9A7) | −0.01536 |
| SLC9A8 | solute carrier family 9 member A8(SLC9A8) | −0.13628 |
| SLC9A9 | solute carrier family 9 member A9(SLC9A9) | −0.33044 |
| SLCO1A2 | solute carrier organic anion transporter family member 1A2(SLCO1A2) | −0.18662 |
| SLCO1B1 | solute carrier organic anion transporter family member 1B1(SLCO1B1) | −0.25209 |
| SLCO1B3 | solute carrier organic anion transporter family member 1B3(SLCO1B3) | −0.05865 |
| SLCO1C1 | solute carrier organic anion transporter family member 1C1(SLCO1C1) | −0.52053 |
| SLCO2A1 | solute carrier organic anion transporter family member 2A1(SLCO2A1) | −0.03198 |
| SLCO2B1 | solute carrier organic anion transporter family member 2B1(SLCO2B1) | −0.21787 |
| SLCO3A1 | solute carrier organic anion transporter family member 3A1(SLCO3A1) | −0.44277 |
| SLCO4A1 | solute carrier organic anion transporter family member 4A1(SLCO4A1) | −0.02087 |
| SLCO4C1 | solute carrier organic anion transporter family member 4C1(SLCO4C1) | −0.2852 |
| SLCO5A1 | solute carrier organic anion transporter family member 5A1(SLCO5A1) | −0.07686 |
| SMC3 | structural maintenance of chromosomes 3(SMC3) | −0.9992 |
| SMOX | spermine oxidase(SMOX) | −0.28547 |
| SMPD1 | sphingomyelin phosphodiesterase 1(SMPD1) | −0.41138 |
| SMPD2 | sphingomyelin phosphodiesterase 2(SMPD2) | −0.30562 |
| SMPD3 | sphingomyelin phosphodiesterase 3(SMPD3) | −0.09491 |
| SMPD4 | sphingomyelin phosphodiesterase 4(SMPD4) | −0.22793 |
| SMPDL3A | sphingomyelin phosphodiesterase acid like 3A(SMPDL3A) | −0.34998 |
| SMPDL3B | sphingomyelin phosphodiesterase acid like 3B(SMPDL3B) | −0.15774 |
| SMS | spermine synthase(SMS) | −0.27179 |
| SOAT1 | sterol O-acyltransferase 1(SOAT1) | −0.25523 |
| SOAT2 | sterol O-acyltransferase 2(SOAT2) | −0.1887 |
| SOD1 | superoxide dismutase 1, soluble(SOD1) | −0.73139 |
| SOD2 | superoxide dismutase 2, mitochondrial(SOD2) | −0.25865 |
| SOD3 | superoxide dismutase 3, extracellular(SOD3) | −0.39856 |
| SORD | sorbitol dehydrogenase(SORD) | −0.02983 |
| SPATA16 | spermatogenesis associated 16(SPATA16) | −0.30742 |
| SPHK1 | sphingosine kinase 1(SPHK1) | −0.18944 |
| SPHK2 | sphingosine kinase 2(SPHK2) | −0.33176 |
| SPNS1 | sphingolipid transporter 1 (putative)(SPNS1) | −0.36655 |
| SPR | sepiapterin reductase (7,8-dihydrobiopterin:NADP+ oxidoreductase)(SPR) | −0.15576 |
| SPTLC1 | serine palmitoyltransferase long chain base subunit 1(SPTLC1) | −0.06161 |
| SPTLC2 | serine palmitoyltransferase long chain base subunit 2(SPTLC2) | 0.014128 |
| SPTLC3 | serine palmitoyltransferase long chain base subunit 3(SPTLC3) | −0.26901 |
| SQLE | squalene epoxidase(SQLE) | −0.315 |
| SQRDL | sulfide quinone reductase-like (yeast) (SQRDL) | −0.35567 |
| SRCAP | Snf2 related CREBBP activator protein(SRCAP) | −0.10086 |
| SRD5A1 | steroid 5 alpha-reductase 1(SRD5A1) | −0.26731 |
| SRFBP1 | serum response factor binding protein 1(SRFBP1) | 0.069773 |
| SRM | spermidine synthase(SRM) | 0.052068 |
| SRSF3 | serine and arginine rich splicing factor 3(SRSF3) | −0.36487 |
| SSH1 | slingshot protein phosphatase 1(SSH1) | 0.154457 |
| SSH2 | slingshot protein phosphatase 2(SSH2) | −0.01103 |
| SSH3 | slingshot protein phosphatase 3(SSH3) | 0.33775 |
| SSU72 | SSU72 homolog, RNA polymerase II CTD phosphatase(SSU72) | 0.474911 |
| STAR | steroidogenic acute regulatory protein(STAR) | −0.08668 |
| STAT3 | signal transducer and activator of transcription 3(STAT3) | −0.12952 |
| STS | steroid sulfatase (microsomal), isozyme S(STS) | 0.032737 |
| STYX | serine/threonine/tyrosine interacting protein(STYX) | −0.02066 |
| STYXL1 | serine/threonine/tyrosine interacting like 1(STYXL1) | −0.26616 |
| SUCLA2 | succinate-CoA ligase ADP-forming beta subunit(SUCLA2) | −0.11532 |
| SUCLG1 | succinate-CoA ligase alpha subunit(SUCLG1) | −0.1976 |
| SUCLG2 | succinate-CoA ligase GDP-forming beta subunit(SUCLG2) | −0.43846 |
| SULF1 | sulfatase 1(SULF1) | −0.18277 |
| SULF2 | sulfatase 2(SULF2) | −0.46576 |
| SULT1A1 | sulfotransferase family 1A member 1(SULT1A1) | −0.15472 |
| SULT1B1 | sulfotransferase family 1B member 1(SULT1B1) | −0.20281 |
| SULT1C4 | sulfotransferase family 1C member 4(SULT1C4) | −0.18778 |
| SULT1E1 | sulfotransferase family 1E member 1(SULT1E1) | 0.21146 |
| SULT2A1 | sulfotransferase family 2A member 1(SULT2A1) | −0.37252 |
| SUOX | sulfite oxidase(SUOX) | −0.08244 |
| SUPT6H | SPT6 homolog, histone chaperone(SUPT6H) | −0.24798 |
| SUPV3L1 | Suv3 like RNA helicase(SUPV3L1) | −0.32928 |
| SURF1 | SURF1, cytochrome c oxidase assembly factor(SURF1) | −0.12504 |
| SV2A | synaptic vesicle glycoprotein 2A(SV2A) | −0.36026 |
| SV2B | synaptic vesicle glycoprotein 2B(SV2B) | −0.29932 |
| SV2C | synaptic vesicle glycoprotein 2C(SV2C) | −0.47623 |
| SYNJ1 | synaptojanin 1(SYNJ1) | −0.12584 |
| SYNJ2 | synaptojanin 2(SYNJ2) | −0.28228 |
| TAAR2 | trace amine associated receptor 2(TAAR2) | −0.3892 |
| TALDO1 | transaldolase 1(TALDO1) | 0.081307 |
| TAP1 | transporter 1, ATP binding cassette subfamily B member(TAP1) | −0.65218 |
| TAP2 | transporter 2, ATP binding cassette subfamily B member(TAP2) | −0.35061 |
| TAS2R50 | taste 2 receptor member 50(TAS2R50) | −0.43089 |
| TAT | tyrosine aminotransferase(TAT) | −0.30423 |
| TBXAS1 | thromboxane A synthase 1(TBXAS1) | −0.15413 |
| TCIRG1 | T-cell immune regulator 1, ATPase H+ transporting V0 subunit a3(TCIRG1) | −0.35642 |
| TCN2 | transcobalamin 2(TCN2) | −0.21074 |
| TDO2 | tryptophan 2,3-dioxygenase(TDO2) | 0.0007 |
| TECR | trans-2,3-enoyl-CoA reductase(TECR) | −0.40084 |
| TENC1 | tensin Like C1 Domain-Containing Phosphatase (TENC1) | −0.05715 |
| TF | transferrin(TF) | −0.36536 |
| TH | tyrosine hydroxylase(TH) | −0.06742 |
| THEM4 | thioesterase superfamily member 4(THEM4) | −1.02617 |
| THNSL1 | threonine synthase like 1(THNSL1) | −0.15419 |
| THNSL2 | threonine synthase like 2(THNSL2) | −0.21678 |
| THOP1 | thimet oligopeptidase 1(THOP1) | −0.34201 |
| THRA | thyroid hormone receptor, alpha(THRA) | 0.025703 |
| THRB | thyroid hormone receptor beta(THRB) | −0.18007 |
| TIMM50 | translocase of inner mitochondrial membrane 50(TIMM50) | −0.04576 |
| TK1 | thymidine kinase 1(TK1) | 0.029589 |
| TK2 | thymidine kinase 2, mitochondrial(TK2) | −0.55631 |
| TKT | transketolase(TKT) | −0.0866 |
| TKTL1 | transketolase like 1(TKTL1) | −0.05799 |
| TKTL2 | transketolase like 2(TKTL2) | −0.20726 |
| TM7SF2 | transmembrane 7 superfamily member 2(TM7SF2) | 0.232688 |
| TMCO3 | transmembrane and coiled-coil domains 3(TMCO3) | −0.15534 |
| TMLHE | trimethyllysine hydroxylase, epsilon(TMLHE) | −0.17346 |
| TPCN1 | two pore segment channel 1(TPCN1) | −0.16721 |
| TPCN2 | two pore segment channel 2(TPCN2) | −0.13385 |
| TPD52L3 | tumor protein D52 like 3(TPD52L3) | −0.14927 |
| TPH1 | tryptophan hydroxylase 1(TPH1) | −0.11949 |
| TPH2 | tryptophan hydroxylase 2(TPH2) | −0.25383 |
| TPI1 | triosephosphate isomerase 1(TPI1) | −0.39885 |
| TPK1 | thiamin pyrophosphokinase 1(TPK1) | −0.58798 |
| TPMT | thiopurine S-methyltransferase(TPMT) | −0.1285 |
| TPO | thyroid peroxidase(TPO) | −0.1711 |
| TPTE | transmembrane phosphatase with tensin homology(TPTE) | −0.08734 |
| TPTE2 | transmembrane phosphoinositide 3-phosphatase and tensin homolog 2(TPTE [symbol] | −0.5281 |
| TRPA1 | transient receptor potential cation channel subfamily A member 1(TRPA1) | −0.1154 |
| TRPC1 | transient receptor potential cation channel subfamily C member 1(TRPC1) | −0.55949 |
| TRPC3 | transient receptor potential cation channel subfamily C member 3(TRPC3) | −0.3319 |
| TRPC4 | transient receptor potential cation channel subfamily C member 4(TRPC4) | −0.04834 |
| TRPC5 | transient receptor potential cation channel subfamily C member 5(TRPC5) | −0.18444 |
| TRPC6 | transient receptor potential cation channel subfamily C member 6(TRPC6) | 0.085471 |
| TRPC7 | transient receptor potential cation channel subfamily C member 7(TRPC7) | 0.271312 |
| TRPM1 | transient receptor potential cation channel subfamily M member 1(TRPM1) | −0.10933 |
| TRPM2 | transient receptor potential cation channel subfamily M member 2(TRPM2) | −0.21687 |
| TRPM3 | transient receptor potential cation channel subfamily M member 3(TRPM3) | 0.007508 |
| TRPM4 | transient receptor potential cation channel subfamily M member 4(TRPM4) | −0.14376 |
| UMPS | uridine monophosphate synthetase(UMPS) | −0.40763 |
| UPB1 | beta-ureidopropionase 1(UPB1) | 0.035896 |
| UPP1 | uridine phosphorylase 1(UPP1) | −0.52046 |
| UPP2 | uridine phosphorylase 2(UPP2) | −0.17921 |
| UPRT | uracil phosphoribosyltransferase homolog(UPRT) | −0.14698 |
| UQCR10 | ubiquinol-cytochrome c reductase, complex III subunit X(UQCR10) | −0.2061 |
| UQCR11 | ubiquinol-cytochrome c reductase, complex III subunit XI(UQCR11) | −0.05471 |
| UQCRB | ubiquinol-cytochrome c reductase binding protein(UQCRB) | −0.33931 |
| UQCRC1 | ubiquinol-cytochrome c reductase core protein I(UQCRC1) | −0.49316 |
| UQCRC2 | ubiquinol-cytochrome c reductase core protein II(UQCRC2) | −0.61236 |
| UQCRFS1 | ubiquinol-cytochrome c reductase, Rieske iron-sulfur polypeptide 1(UQCRFS1 [symbol] | −0.02722 |
| UQCRH | ubiquinol-cytochrome c reductase hinge protein(UQCRH) | −0.32743 |
| UROD | uroporphyrinogen decarboxylase(UROD) | −0.29562 |
| UROS | uroporphyrinogen III synthase(UROS) | −0.46661 |
| USP39 | ubiquitin specific peptidase 39(USP39) | −0.65534 |
| VCP | valosin containing protein(VCP) | −0.208 |
| VDAC1 | voltage dependent anion channel 1(VDAC1) | −0.3887 |
| VDAC2 | voltage dependent anion channel 2(VDAC2) | −0.22048 |
| VDAC3 | voltage dependent anion channel 3(VDAC3) | −0.05312 |
| VDR | vitamin D (1,25-dihydroxyvitamin D3) receptor(VDR) | 0.05968 |
| WDR60 | WD repeat domain 60(WDR60) | −0.04696 |
| WFDC11 | WAP four-disulfide core domain 11(WFDC11) | −0.47063 |
| WWOX | WW domain containing oxidoreductase(WWOX) | −0.14108 |
| XDH | xanthine dehydrogenase(XDH) | −0.11757 |
| XPO1 | exportin 1(XPO1) | −0.26682 |
| XYLB | xylulokinase(XYLB) | −0.15293 |
| YY1 | YY1 transcription factor(YY1) | 0.093575 |
| ZACN | zinc activated ion channel(ZACN) | −0.01557 |
| ZADH2 | zinc binding alcohol dehydrogenase domain containing 2(ZADH2) | −0.20458 |
| ZC3H13 | zinc finger CCCH-type containing 13(ZC3H13) | −0.28188 |
| ZNF207 | zinc finger protein 207(ZNF207) | −0.07154 |

[symbol]

indicates data missing or illegible when filed

1. A method of treating a cancer in a subject, the method comprising administering to the subject a therapeutically effective amount of at least one ABL inhibitor and at least one mevalonate pathway inhibitor such that the cancer is treated in the subject.

2. The method of claim 1, wherein the cancer is a metastatic cancer.

3. The method of claim 1, wherein the ABL inhibitor ABL-001, imatinib, nilotinib, dasatinib (BMS-354825), bosutinib (SKI-606), Ponatinib (AP24534), Bafetinib (INNO-406), axitinib, vandertanib, GNF2, GNF5, HG-7-85-01, Tozasertib (MK-0457, VX-680), Danusertib (PHA-739358), Rebastinib (DCC-2036), 1,3,4-thiadiazole derivatives, Compound 2 having the structure 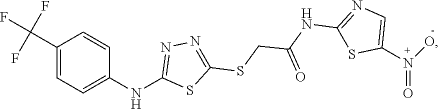
or pharmaceutically acceptable salts of any thereof.

4. The method of claim 1, wherein the at least one mevalonate pathway inhibitor is a cholesterol biosynthesis inhibitor.

5. The method of claim 4, wherein the cholesterol biosynthesis inhibitor comprises a statin.

6. The method of claim 4, wherein the statin comprises a comprises a lipophilic statin.

7. The method of claim 5, wherein the statin is selected from the group consisting of: simvastatin, atorvastatin, lovastatin, pravastatin, Fluvastatin, rosuvastatin, pitavastatin, and combinations of any thereof.

8. The method of claim 1, wherein the at least one mevalonate pathway inhibitor comprises a prenylation inhibitor.

9. The method of claim 8, wherein the prenylation inhibitor comprises at least one of geranylgeranyl transferase-1 (GGT-1) inhibitor GGTI-298 or farnesyl transferase (FT) inhibitor FTI-277.

10. The method of claim 1, wherein the at least one ABL kinase inhibitor is administered prior to the at least one mevalonate pathway inhibitor.

11. The method of claim 1, wherein the at least one ABL kinase inhibitor is administered concurrently with the at least one mevalonate pathway inhibitor.

12. The method of claim 1, wherein the at least one ABL kinase inhibitor is administered after the at least one mevalonate pathway inhibitor.

13. The method of claim 1, wherein the subject is also treated with at least one of an anti-cancer agent or radiotherapy.

14. The method of claim 13, wherein the anti-cancer agent comprises one or more of a chemotherapeutic agent, a tyrosine kinase inhibitor, or an immunotherapeutic agent.

15. The method of claim 1, wherein the subject has lung cancer, breast cancer, or skin cancer.

16. The method of claim 1, wherein the at least one ABL inhibitor comprises an allosteric ABL inhibitor.

17. The method of claim 1, wherein the at least one ABL inhibitor comprises GNF5 or ABL001.

18. The method of claim 1, wherein the at least one mevalonate pathway inhibitor comprises simvastatin or fluvastatin.

19. The method of claim 1, wherein the at least one ABL inhibitor comprises ABL001 and the at least one mevalonate pathway inhibitor comprises simvastatin.

20. The method of claim 2, wherein the metastatic cancer comprises metastases in the brain of the subject.


SEQUENCE LISTING (13 sequences)

SEQ ID NO: 1 - DNA, 21 bp, Artificial Sequence
  Description of Artificial Sequence Synthetic primer
gacctcaacgcacagtacgag

SEQ ID NO: 2 - DNA, 22 bp, Artificial Sequence
  Description of Artificial Sequence Synthetic primer
aggagtcccatgatgagattgt

SEQ ID NO: 3 - DNA, 19 bp, Artificial Sequence
  Description of Artificial Sequence Synthetic primer
ggtggggtcatgtgtgtgg

SEQ ID NO: 4 - DNA, 22 bp, Artificial Sequence
  Description of Artificial Sequence Synthetic primer
cggttcaggtactcagtcatcc

SEQ ID NO: 5 - DNA, 21 bp, Artificial Sequence
  Description of Artificial Sequence Synthetic primer
gagctggtggttgactttctc

SEQ ID NO: 6 - DNA, 21 bp, Artificial Sequence
  Description of Artificial Sequence Synthetic primer
tccatctccgattcagtccct

SEQ ID NO: 7 - DNA, 21 bp, Artificial Sequence
  Description of Artificial Sequence Synthetic primer
tgtgggcatcaatggatttgg

SEQ ID NO: 8 - DNA, 22 bp, Artificial Sequence
  Description of Artificial Sequence Synthetic primer
acaccatgtattccgggtcaat

SEQ ID NO: 9 - DNA, 21 bp, Artificial Sequence
  Description of Artificial Sequence Synthetic shRNA
ggtgtatgggctactatagaa

SEQ ID NO: 10 - DNA, 21 bp, Artificial Sequence
  Description of Artificial Sequence Synthetic shRNA
ggtgtatgagctgctagagaa

SEQ ID NO: 11 - DNA, 21 bp, Artificial Sequence
  Description of Artificial Sequence Synthetic shRNA
ccttatctcacccactctgaa

SEQ ID NO: 12 - DNA, 58 bp, Artificial Sequence
  Description of Artificial Sequence Synthetic shRNA
ccgggcagtgataaaggaggcatttctcgagaaatgcctcctttatcactgctttttg

SEQ ID NO: 13 - DNA, 58 bp, Artificial Sequence
  Description of Artificial Sequence Synthetic shRNA
ccggcctgctgctataaattggatactcgagtatccaatttatagcagcaggtttttg